(19) United States
(12) Patent Application Publication
(10) Pub. No.: US 2005/0070524 A1
(43) Pub. Date: Mar. 31, 2005

(54) Compositions of a cyclooxygenase-2 selective inhibitor and an anticonvulsant agent for the treatment of central nervous system disorders

(75) Inventors: Diane T. Stephenson, Groton, CT (US); Duncan P. Taylor, Bridgewater, NJ (US); Stephen P. Arneric, Milan, MI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/862,846

(22) Filed: Jun. 7, 2004

Related U.S. Application Data
(60) Provisional application No. 60/476,575, filed on Jun. 6, 2003.

(51) Int. Cl. A61K 31/55; A01N 43/58; A61K 31/515; A61K 31/19; A61K 31/415

(52) U.S. Cl. 514/217; 514/406; 514/247; 514/221; 514/270; 514/557

(57) ABSTRACT

The present invention provides compositions and methods for the treatment of central nervous system disorders or related conditions in a subject. More particularly, the invention provides a combination therapy for the treatment of seizures, or seizure disorders comprising the administration to a subject of an anticonvulsant agent in combination with a cyclooxygenase-2 selective inhibitor.

### CROSS REFERENCE TO RELATED APPLICATION

This application claims priority from Provisional Application Ser. No. 60/476,575 filed on Jun. 6, 2003, which is hereby incorporated by reference in its entirety.

### FIELD OF THE INVENTION

The present invention provides compositions and methods for the treatment of central nervous system (CNS) disorders or related conditions. More particularly, the invention is directed toward a combination therapy for the treatment or prevention of seizures or seizure disorders, comprising the administration to a subject of an anticonvulsant agent in combination with a cyclooxygenase-2 selective inhibitor.

### BACKGROUND OF THE INVENTION

The predominant application of anticonvulsant agents is their use in the treatment or control of seizures or convulsions. Seizures are the result of a sudden disruption of the brain's normal electrical functions, resulting from the abnormal firing of populations of neurons in the brain. Seizures may result in a variety of neurological and behavioral manifestations, including sensory, motor, or autonomic disturbances, and an altered state of consciousness. Seizures can last from a few seconds to a few minutes, and result from a variety of causes including infection in the brain (e.g. meningitis), diabetes, high fever, brain tumor, serious head trauma, or poisoning, among others.

About 10% of Americans will experience some type of seizure during their lifetime. Of those, about 3% will be diagnosed with epilepsy. Epilepsy is a seizure disorder characterized by recurring unprovoked seizures that have no single identifiable underlying cause. There are two main types of epilepsy-generalized epilepsy, which results when abnormal electrical activity exists throughout the brain, and partial (or focal) epilepsy, which results when the abnormal electrical activity begins focally in a particular area of the brain. Neither generalized nor partial epilepsy is associated with one single type of seizure; epileptics may, and often do, suffer from more than one seizure type.

The symptoms associated with seizures vary depending on the area of the brain affected by the abnormal electrical activity. Seizures may occur as a single event, unrelated to epilepsy, or they may recur as a symptom of generalized or partial epilepsy. Seizures associated with generalized epilepsy include absence (petit mal) seizures, myoclonic seizures, clonic seizures, tonic seizures, tonic-clonic (grand mal) seizures, and atonic seizures. Generalized seizures may be either convulsive or nonconvulsive, but they always involve a loss of consciousness. Absence seizures, most common in children under 20, are characterized by an abrupt, short-term lack of conscious activity, typically lasting only a few seconds. Absence seizures often manifest themselves as involuntary staring episodes, during which the child has no conscious awareness of surroundings, and all conscious activity, such as movement or talking ceases. Tonic-clonic (grand mal) seizures may affect people of every age, and typically involve the entire body. They may result in muscle rigidity, violent rhythmic muscle contractions, and loss of consciousness.

Seizures associated with partial epilepsy include simple partial (focal) seizures, complex partial seizures, and secondarily generalized tonic-clonic seizures. The symptoms arising from a partial seizure depend upon which part of the brain is affected. For example, partial (focal) seizures may result from abnormal electrical activity in the motor and sensory areas of the cerebral cortex. They typically involve rhythmic muscle contractions or abnormal sensations (such as numbness, tingling, etc.) in an isolated area of the body, but do not involve a loss of consciousness. Complex partial seizures are often the result of abnormal electrical activity in the temporal lobes of the brain. They are characterized by a change in alertness or awareness along with a temporary loss of memory and various behavioral or emotional symptoms, such as automatism or sensory hallucinations. Both partial seizures and complex partial seizures may occur at any age. See, e.g., Katzung, B. G., ed., Basic and Clinical Pharmacology, 8^{th} ed., McGraw-Hill, Inc. (2001), p. 413-14 (discussing the seizure classification system).

Seizures are commonly treated with one or more anticonvulsant agent. These agents may exert their anticonvulsive effect through a variety of complex mechanisms of action. Without wishing to be bound to any particular theory, many anticonvulsants are thought to act through one or more of the following mechanisms, involving: 1) mediation of voltage-sensitive ion channels; 2) direct or indirect actions involving a gamma aminobutyric acid (GABA) or the GABA_{A} receptor; and 3) inhibition of excitatory amino acids (e.g. glutamate or aspartate, among others) by acting as an excitatory amino acid (EAA) receptor antagonists. However, some clinically effective anticonvulsant agents do not have a known mechanism of action.

Some anticonvulsants stabilize hyperexcited neurons and inhibit the repetitive neuronal firing associated with seizures by blocking voltage-sensitive ion channels (e.g. sodium or calcium). Seizures occur when neurons undergo depolarization and fire action potentials at a high frequency. Depolarization of the neuron triggers the opening of ion channels, which is required for an action potential. The channels spontaneously close after opening (called inactivation), and must recover from inactivation before they may participate in another action potential. Some anticonvulsants act by decreasing the rate of recovery of the ion channels from inactivation, thus limiting the ability of the neuron to fire at a high frequency. For example, a study by Stefani, et al., found that the anticonvulsant drug lamotrigine produced a large dose-dependent inhibition of high-voltage-activated Ca²⁺ currents in rat cortical neurons (Stefani, et al., (1996) Eur. J. Pharmacol. 307(1):113-6). A study by Lukyanetz, et al., found that the anticonvulsant levetiracetam directly and irreversibly inhibited the high-voltage-activated Ca²⁺ channels in isolated CA1 hippocampal neurons of rats (Lukyanetz, et al., (2002) Epilepsia 43(1):9-18). Other examples of anticonvulsant agents thought to act on either Na⁺ or Ca²⁺ channels by limiting the rate of recovery from inactivation include, for example, carbamazepine, phenytoin, topiramate, valproate, and zonisamide, among others.

Gamma aminobutyric acid (GABA) is a major inhibitory neurotransmitter involved in the regulation of brain neuronal activity. Some anticonvulsant agents exert their anticonvulsive effect by acting either directly or indirectly to mediate the action of the (GABA)_{A} receptor, a ligand-gated Cl⁻ channel in the central nervous system (CNS). When GABA interacts with the GABA_{A} neuronal receptor, the membrane channel opens, allowing chloride ions to flow into neurons. This in turn reduces the ability of neurons to depolarize to the threshold potential necessary to produce action potentials. An increased level of available GABA is thus desirable for the treatment of seizures. Some anticonvulsant agents (e.g. benzodiazepines and barbiturates) enhance GABA_{A} receptor mediated inhibition through distinct actions on the GABA_{A} receptor. For example, benzodiazepines act as positive modulators by interacting with the benzodiazepine recognition site located on the GABA_{A} receptor, thus increasing the frequency with which the GABA activated Cl⁻ channels open. Other anticonvulsant agents, such as vigabatrin, act by increasing the amount of GABA available for synaptic release by irreversibly inhibiting enzymes that degrades GABA. See, e.g., Goodman and Gilman's The Pharmacological Basis of Therapeutics, 10^{th} ed., McGraw-Hill (2001), pp. 525-26.

Glutamate is one of the predominant excitatory amino acids (EAA) in the CNS. As such, glutamate receptors are important in excitatory neurotransmission. There are several distinct types of glutamate receptors including, for example, AMPA receptors, kainic acid (KA) receptors, and NMDA receptors. In particular, NMDA receptor activation is thought to play a major role in the occurrence of some types of epileptic seizures as well as a variety of other CNS disorders. See, e.g., DeLorenzo, et al., (1998) Proc. Natl. Acad. Sci. USA, 95:14482-87, 14487 (“NMDA receptor-mediated elevations in [Ca²⁺]ᵢ, were shown to induce ‘epilepsy.’”). Thus, certain NMDA receptor antagonists may have an important role in the treatment and control of seizures as well as a variety of neurodegenerative disorders. The NMDA receptor complex contains a number of distinct binding sites, recognizing the excitatory amino acids glutamate and glycine, and the synthetic compound NMDA, among others. Activation of the receptor by glutamate opens an ion channel, allowing an inflow of calcium and sodium ions, among others, which results in the firing of an action potential. Some events, such as various types of CNS trauma or stroke, result in the release of excessive amounts of glutamate, which results in persistent activation of the NMDA receptor. This hyper-excitation can result in cell death (“excitotoxicity”), and has been associated with a variety of disorders, including epilepsy, neurodegenerative diseases (e.g. Alzheimer's disease, Huntington's disease, and Parkinson's disease), stroke, and neuropathic pain and anxiety. The competitive binding of NDMA antagonists at the glutamate recognition site can stop the effects of glutamate activation. Felbamate is one such NMDA receptor antagonist used in the treatment of seizures. Other NMDA antagonists may also be useful in the treatment of seizures and NMDA related disorders and conditions. See, e.g., U.S. Pat. No. 6,406,716 (NMDA antagonists in combination with anticonvulsants for the treatment of neuropathic pain).

The selection of an appropriate anticonvulsant agent or agents for the treatment of a particular patient is often a complex process, depending on several factors. For example, anticonvulsants effective for the control of one type of seizure may be ineffective, or even worsen the occurrence of other types of seizures. Anticonvulsants may also be more or less effective in seizure control depending on the age of the patient. In addition, drug interactions and side effects commonly occur with anticonvulsants. The choice of anticonvulsants for seizure treatment is therefore complex, depending on the patient and seizure type, as well as potential drug side effects and interactions between the co-administered anticonvulsants. In addition, some patients remain refractory to currently available therapies.

The anticonvulsants carbamazepine, valproate, phenytoin, and phenobarbital, among others, have traditionally been used as a first line treatment for partial seizures and generalized tonic-clonic seizures. The anticonvulsants known as the succinimides (e.g. ethosuximide and methsuximide) are commonly prescribed to control absence seizures. However, these drugs have been found to be ineffective in treating many individuals suffering from seizures, due in part to the serious side effects that are often associated with these anticonvulsant agents. For example, side effects associated with these traditional anticonvulsants include, among others, drowsiness, headache, blurred vision, and nausea (carbamazepine); sedation, depression, and slowing of cognitive functions (phenobarbital); drowsiness, gingival hyperplasia, and thickening of facial features (phenytoin); weight gain, gastrointestinal distress, and tremor (valproate); and nausea, vomiting, drowsiness, unsteadiness, and behavior changes (succinimides).

Although known for their anti-anxiety effects, benzodiazepines such as clobazam, clonazepam, clorazepate, lorazepam, nitrazepam, and diazepam are also prescribed as an add-on treatment for a variety of different seizure types, including Lennox-Gastaut syndrome, atonic and myoclonic seizures, and absence seizures. However, side effects associated with benzodiazepines can also be serious, and include drowsiness, slurred speech, behavior changes, and dizziness, among others. Problems with tolerance and dependence have also been associated with the benzodiazepines.

In addition to potentially serious side effects, traditional anticonvulsants are also prone to drug interactions. Drug interactions can affect the plasma concentration of an anticonvulsant, decreasing the effectiveness of the anticonvulsant agent or resulting in toxicity to the patient. For example, some drugs (e.g. carbamazepine, phenobarbital, primidone, and phenytoin) are enzyme-inducing drugs that readily enhance the synthesis of enzymes involved in the metabolism of various other anticonvulsants (e.g. lamotrigine, topiramate, and tiagabine) as well as many other drugs (e.g. oral contraceptives and antidepressants). This results in a reduction of the plasma concentration and thus the effectiveness of the affected drugs. Other drugs, such as sodium valproate, are enzyme inhibitors. These drugs can reduce the clearance rate of various anticonvulsants, resulting in an increase in plasma concentration and possible toxicity to the patient. Such enzyme inhibitors commonly affect phenytoin, carbamazepine, and lamotrigine. In addition, some anticonvulsants (e.g. phenytoin) are highly bound to plasma proteins. The plasma concentration of these anticonvulsant agents is affected when the competitive binding of other drugs displaces them from their plasma-binding site. The dosage and serum level of anticonvulsants must thus be closely monitored when they are co-administered with other drugs. For a further discussion of these and some other common anticonvulsant drug interactions, see, e.g., Patsalos, et al., Epilepsia (2002), 43(4):365-85; Brown, Pharm. J. (1999), 262(7035):325-27.

Recently, newer anticonvulsants have been introduced in an attempt to increase the effectiveness of seizure control and reduce the problems of adverse side effects and drug interactions associated with traditional anticonvulsants. Gabapentin, lamotrigine, topiramate, tiagabine, levetiracetam, oxcarbazepine, felbamate, and zonisamide are all prescribed as either add-on or monotherapy for partial seizures. Felbamate, gabapentin, lamotrigine, and topiramate are also being investigated for their efficacy in treating other types of seizures.

Although some improvements have been made in terms of drug interactions and tolerability, the newer anticonvulsants have not eliminated the problems associated with the traditional anticonvulsants. Drug interactions are still problematic, especially when these newer anticonvulsants are co-administered with the traditional anticonvulsants. In addition, side effects ranging from drowsiness and fatigue, to dizziness, nausea, and headache, among others, are fairly common. More serious rare or idiosyncratic side effects, including severe rash (lamotrigine), nephrolithiasis (topiramate), and hepatic failure and anorexia (felbamate) may also occur. In addition, not all patients are seizure free, even with treatment with anticonvulsants. It is thus clear that there is still a need for novel and/or improved treatments for seizures and seizure disorders.

Many anticonvulsants are now being explored for their effectiveness in treating or controlling a variety of other disorders and conditions in addition to seizures. Included among these are various psychiatric disorders, neurodegenerative disorders, migraine, and neuropathic pain. For example, the anticonvulsants carbamazepine and valproate (i.e. divalproex sodium or valproic acid) are mood stabilizers, and are sometimes prescribed for the treatment of the mood disorders mania or bipolar disorder. Recently, the effectiveness of anticonvulsants such as tiagabine, zonisamide, oxcarbazepine, lamotrigine, gabapentin, and topiramate in the maintenance or acute treatment of mania and bipolar disorder has also been investigated. See, e.g., U.S. Pat. Nos. 5,753,693 (topiramate) and 5,914,333 (tiagabine); De León, Harv. Rev. Psych. (2001), 9:209-22. Other anticonvulsants, such as lamotrigine, topiramate, and gabapentin, may have some effectiveness in treating bipolar or possibly unipolar depression. See, e.g. U.S. Pat. App. No. 2002/0094960 (topiramate). Various benzodiazepines, known for their anti-anxiety effects, have also been used as an adjunct treatment for mania to rapidly treat manic symptoms and calm patients until mood-stabilizing agents can take effect. Benzodiazepines in particular have been used to treat anxiety, anxiety disorders, and sleep disorders such as insomnia. In addition, certain anticonvulsants may also be used to help control mood, anxiety, or agitation associated with other psychiatric disorders. For example, benzodiazepines, carbamazepine, valproate, topiramate, and gabapentin may be used to treat agitation in patients suffering from dementia, psychosis, personality disorders, or other disorders. See also U.S. Pat. No. 6,420,369 (describing the use of topiramate and related anticonvulsants to treat anxiety and psychotic disturbances in dementia patients). Anxiety disorders, as well as other psychiatric disorders, are described in the American Psychiatric Association Diagnostic and Statistical Manual, 4^{th} ed. (DSM-IV). Other anticonvulsants, such as valproate, topiramate, and gabapentin may also be useful in the prevention of migraine. See, e.g., U.S. Pat. Nos. 5,998,380 (topiramate); 5,929,061 and 5,767,117 (tiagabine, vigabatrin, benzodiazepines); 6,323,365 (valproic acid derivatives). Valproate in particular has been approved as a preventative treatment for migraine.

The use of a variety of different anticonvulsants in the treatment of other disorders has also been described. U.S. Pat. Nos. 5,929,065 (tiagabine and vigabatrin) and 6,372,792 (gabapentin) describe the use of various anticonvulsants in the treatment of sleep disorders and anxiety and anxiety disorders such as panic. U.S. Pat. Nos. 5,028,611 (carbamazepine), 6,426,368 (gabapentin and related compounds), and 5,102,913 (valproic acid and derivatives) describe the use of various anticonvulsants in the treatment of withdrawal symptoms from substance abuse. Vigabatrin, lamotrigine, and various benzodiazepines may also be used to treat withdrawal symptoms. U.S. Pat. Nos. 5,084,479 (gabapentin); 5,658,900 (carbamazepine and oxcarbazepine); 5,753,694 (topiramate); 6,133,299; and 6,342,515 (zonisamide) describe the use of various anticonvulsants in the treatment of neurodegenerative diseases such as Parkinson's disease, Huntington's disease, Alzheimer's disease, and amyotrophic lateral sclerosis (ALS), as well as stroke, head trauma, and asphyxia. U.S. Pat. Nos. 6,071,537 and 6,191,117 describe the use of various anticonvulsants in the treatment of obesity. U.S. Pat. Nos. 4,992,443 and 5,234,929 describe the use of various anticonvulsants in the treatment of motion sickness. The use of various anticonvulsants in the treatment of vertigo and psoriasis are described in U.S. Pat. Nos. 6,333,352 (vertigo) and 5,760,006 (psoriasis). U.S. Pat. Nos. 6,191,163; 6,201,010; 6,214,867; 6,362,220; 2002/0006908; and 6,319,903 describe the use of topiramate and related compounds to lower lipids, lower blood pressure, treat essential tremor, decrease blood glucose levels, treat schizophrenia, and treat cluster headaches, respectively.

In addition, the similarity between some models of epilepsy and some neuropathic pain models has led to the investigation of anticonvulsants in the treatment of neuropathic pain. See, e.g., U.S. Pat. Nos. 6,191,142; 6,369,228; 5,760,007; and 5,935,933. Neuropathic pain is a type of chronic pain likely resulting from neuronal hyperexcitability in diseased or injured areas of the central or peripheral nervous system. Carbamazepine may be useful in relieving diabetic neuropathy, trigeminal neuralgia, and postherpetic neuralgia; gabapentin may be useful in relieving diabetic neuropathy, mixed neuropathies, and postherpetic neuralgia, and allodynia and hyperalgesia in animal models; lamotrigine may be useful in relieving trigeminal neuralgia, peripheral neuropathy, and post stroke pain. Phenobarbital, clonazepam, valproic acid, topiramate, and tiagabine may also have effects on neuropathic pain.

Anticonvulsants may thus have efficacy in the treatment and control of a wide variety of central nervous system associated disorders. However, because of side effects and drug interactions associated with anticonvulsant agents, new and/or improved treatments are still needed.

The central nervous system is one of the few tissues that constitutively expresses cyclooxygenase-2 (COX-2) activity. COX-2 mRNA and protein is expressed in neurons and its expression is regulated by neuronal activity. Yamagata, et al., Neuron (1993), 11:371-86. Thus, COX-2 has been named an immediate early gene. COX-2 expression is markedly and transiently upregulated in neurons in response to excitatory stimuli such as seizures and kainate. Id.; Chen, et al., Neuroreport (1995), 6:24548; Tocco, et al., Exp. Neurol. (1997), 144:339-49. Although it has been postulated that COX-2 plays a role in seizure activity, few studies have examined the role of inhibiting COX-2 activity in these models. Some controversy exists as to the role of COX-2 inhibitors in the kainate induced model of seizures. For example, two studies have reported that COX-2 inhibition is beneficial, see, e.g., Ciceri, et al., J. Pharm. Exp. Ther. (2002), 302:846-52; Kunz and Oliw, Eur. J. Neurosci. (2001), 13:569-75, while one study suggests a detrimental effect, see Baik, et al., Brain Res. (1999), 843:118-29. A more direct cause-effect relationship between neuronal COX-2 expression and excitotoxicity was revealed in a study that reported that COX-2 overexpressing transgenic mice exhibit potentiated seizures and lethality when challenged with kainic acid. Kelley, et al., Am. J. Pathol. (1999), 155:995-1004.

The present invention provides an improvement in the treatment of seizures or seizure disorders, as well as other CNS disorders or related conditions for which anticonvulsant agents show a beneficial effect, by providing a combination therapy comprised of an anticonvulsant agent with a cyclooxygenase-2 (COX-2) selective inhibitor. In addition, a therapy for the treatment of seizures or seizure disorders is provided, comprising the administration to a subject of a COX-2 selective inhibitor as monotherapy.

### SUMMARY OF THE INVENTION

Among the several aspects of the invention are a method and a composition for the treatment or control of seizures or seizure disorders in a subject. The composition comprises a cyclooxygenase-2 selective inhibitor or a pharmaceutically acceptable salt or prodrug thereof and an anticonvulsant agent, and the method comprises administering to the subject a cyclooxygenase-2 selective inhibitor or a pharmaceutically acceptable salt or prodrug thereof in combination with an anticonvulsant agent.

In one embodiment, the cyclooxygenase-2 selective inhibitor is a member of the chromene class of compounds. For example, the chromene compound may be a compound of the formula: 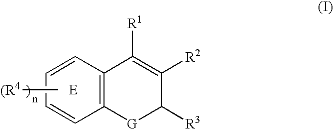
wherein:
n is an integer that is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or wherein R⁴ together with the carbon atoms to which it is attached and the remainder of ring E forms a naphthyl radical.

In another embodiment, the cyclooxygenase-2 selective inhibitor or a pharmaceutically acceptable salt or a prodrug thereof comprises a compound of the formula: 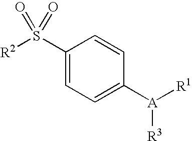
wherein
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and
R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

In another embodiment, the anticonvulsant agent is selected from the group consisting of carbamazepine, clobazam, oxcarbazepine, primidone, acetazolamide, felbamate, gabapentin, lamotrigine, pregabalin, levetiracetam, ralitoline, tiagabine, topiramate, vigabatrin, and zonisamide.

Other aspects of the invention are described in more detail below.

Abbreviations and Definitions

The term “acyl” is a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, and trifluoroacetyl.

The term “alkenyl” is a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The terms “alkenyl” and “lower alkenyl” also are radicals having “cis” and “trans” orientations, or alternatively, “E” and “Z” orientations. The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” are linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” is an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxycarbonyl” is a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The term “anticonvulsant agent” is intended to include prodrugs and pharmaceutically acceptable salts, isomers, esters, derivatives, analogs, and/or other related compounds useful in the treatment of seizures or convulsions. An anticonvulsant agent is useful in the treatment or prevention of CNS disorders or related conditions, where the anticonvulsant agent reduces the duration, severity, or frequency of occurrence of the disorder or symptoms associated with the disorder in a subject in need of such treatment.

Where used, either alone or within other terms such as “haloalkyl”, “alkylsulfonyl”, “alkoxyalkyl” and “hydroxyalkyl”, the term “alkyl” is a linear, cyclic or branched radical having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkylamino” is an amino group that has been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “alkylaminoalkyl” is a radical having one or more alkyl radicals attached to an aminoalkyl radical.

The term “alkylaminocarbonyl” is an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “alkylthio” is a radical containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” is a radical containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” is a radical containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “alkynyl” is a linear or branched radical having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “aminoalkyl” is an alkyl radical substituted with one or more amino radicals. More preferred are “lower aminoalkyl” radicals. Examples of such radicals include aminomethyl, aminoethyl, and the like.

The term “aminocarbonyl” is an amide group of the formula —C(═O)NH2.

The term “aralkoxy” is an aralkyl radical attached through an oxygen atom to other radicals.

The term “aralkoxyalkyl” is an aralkoxy radical attached through an oxygen atom to an alkyl radical.

The term “aralkyl” is an aryl-substituted alkyl radical such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “aralkylamino” is an aralkyl radical attached through an amino nitrogen atom to other radicals. The terms “N-arylaminoalkyl” and “N-aryl-N-alkyl-aminoalkyl” are amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aralkylthio” is an aralkyl radical attached to a sulfur atom.

The term “aralkylthioalkyl” is an aralkylthio radical attached through a sulfur atom to an alkyl radical.

The term “aroyl” is an aryl radical with a carbonyl radical as defined above. Examples of aroyl include benzoyl, naphthoyl, and the like and the aryl in said aroyl may be additionally substituted.

The term “aryl”, alone or in combination, is a carbocyclic aromatic system containing one, two or three rings wherein such rings may be attached together in a pendent manner or may be fused. The term “aryl” includes aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “arylamino” is an amino group, which has been substituted with one or two aryl radicals, such as N-phenylamino. The “arylamino” radicals may be further substituted on the aryl ring portion of the radical.

The term “aryloxyalkyl” is a radical having an aryl radical attached to an alkyl radical through a divalent oxygen atom.

The term “arylthioalkyl” is a radical having an aryl radical attached to an alkyl radical through a divalent sulfur atom.

The term “carbonyl”, whether used alone or with other terms, such as “alkoxycarbonyl”, is —(C═O)—.

The terms “carboxy” or “carboxyl”, whether used alone or with other terms, such as “carboxyalkyl”, is —CO2H.

The term “carboxyalkyl” is an alkyl radical substituted with a carboxy radical. More preferred are “lower carboxyalkyl” which are lower alkyl radicals as defined above, and may be additionally substituted on the alkyl radical with halo. Examples of such lower carboxyalkyl radicals include carboxymethyl, carboxyethyl and carboxypropyl.

The phrase “CNS disorder or related condition” refers to a number of CNS disorders or related conditions on which an anticonvulsant agent may have a beneficial effect when administered as either monotherapy or as adjunct treatment. Such disorders include, but are not limited to various psychiatric disorders, neurodegenerative disorders, and withdrawal symptoms.

The term “control” includes preventing altogether the onset of a clinically evident seizure, seizure disorder, or disorder treatable by an anticonvulsant agent. In the case of seizures or seizure disorders, “control” also includes reducing the frequency or severity of the occurrence of seizures. This definition includes prophylactic treatment.

The term “cycloalkenyl” is a partially unsaturated carbocyclic radical having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “cyclooxygenase-2 selective inhibitor” is a compound able to inhibit cyclooxygenase-2 without significant inhibition of cyclooxygenase-1. Typically, it includes compounds that have a cyclooxygenase-2 IC₅₀ of less than about 0.2 micro molar, and also have a selectivity ratio of cyclooxygenase-2 inhibition over cyclooxygenase-1 inhibition of at least 50, and more typically, of at least 100. Even more typically, the compounds have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “generalized seizure” refers to seizures caused by or associated with a sudden disruption of the brain's normal electrical functions, resulting from abnormal electrical discharges occurring throughout the brain. Generalized seizures include, without limitation, absence (petit mal) seizures, myoclonic seizures, clonic seizures, tonic seizures, tonic-clonic (grand mal) seizures, and atonic seizures. These seizures may occur as a single event, unrelated to epilepsy, or they may be recurring as a result of generalized epilepsy.

The term “halo” is a halogen such as fluorine, chlorine, bromine or iodine.

The term “haloalkyl” is a radical wherein any one or more of the alkyl carbon atoms is substituted with halo as defined above. Specifically included are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” is a radical having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaryl” is an unsaturated heterocyclyl radical. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also includes radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “heterocyclyl” is a saturated, partially unsaturated and unsaturated heteroatom-containing ring-shaped radical, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heterocyclylalkyl” is a saturated and partially unsaturated heterocyclyl-substituted alkyl radical, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “hydrido” is a single hydrogen atom (H). This hydrido radical may be attached, for example, to an oxygen atom to form a hydroxyl radical or two hydrido radicals may be attached to a carbon atom to form a methylene (—CH2—) radical.

The term “hydroxyalkyl” is a linear or branched alkyl radical having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “partial seizure” or “focal seizure” used interchangeably refers to seizures caused by or associated with a sudden disruption of the brain's normal electrical functions, resulting from abnormal electrical discharges occurring in a particular area of the brain. Partial or focal seizures include, without limitation, simple partial (focal) seizures, complex partial seizures, and secondarily generalized tonic-clonic seizures. These seizures may occur as a single event, unrelated to epilepsy, or they may be recurring as a result of partial epilepsy.

The term “seizure” or “convulsion,” used interchangeably, refers to a sudden disruption of the brain's normal electrical functions, resulting from abnormal electrical discharges in the brain. As defined herein, seizures may result in a variety of neurological and behavioral manifestations, including sensory, motor, or autonomic disturbances, and an altered state of consciousness. The term “seizures” includes, but is not limited to, generalized seizures, motor attacks, sensory seizures, visual seizures, auditory seizures, atonic seizures, tonic seizures, clonic seizures, generalized tonic-clonic (grand-mal) seizures, primary generalized seizures, absence (petit mal) seizures, myoclonic seizures, jacksonian seizure, akinetic seizures, simple partial seizures, complex partial seizures, and secondarily generalized seizures. The term “seizure” includes seizures resulting from any identifiable or unidentifiable cause, as well as seizures that occur as a single episode or seizures that recur as part of a chronic seizure disorder such as epilepsy.

The term “seizure disorder” includes any disorder characterized by recurring seizures and/or convulsions. In one embodiment, the seizure disorder is epilepsy.

The term “subject” or “patient,” used interchangeably, for purposes of treatment includes any human or animal subject who is afflicted with or predisposed to have a seizure, seizure disorder, or any disorder treatable by an anticonvulsant agent. The subject can be a domestic livestock species, a laboratory animal species, a zoo animal, or a companion animal. In one embodiment, the subject is a mammal. In one embodiment, the mammal is a human being. A subject is predisposed to have a seizure if the subject suffers from or has been diagnosed with any ailment or condition known to be a cause of seizures including, but not limited to, epilepsy, a family history of epilepsy or seizures, developmental or genetic conditions present at birth, febrile convulsions, metabolic abnormalities (including kidney failure, electrolyte imbalances, phenylketonuria, use of alcohol or drugs, and nutritional deficiencies, etc.), brain injury, tumors and brain lesions that occupy space (e.g. hematomas, etc.), disorders affecting blood vessels (e.g. stroke, etc.), degenerative disorders affecting the brain, or infections (including meningitis, encephalitis, brain abscess, mumps, etc.).

The term “sulfonyl”, whether used alone or linked to other terms such as alkylsulfonyl, is a divalent radical —SO₂—. “Alkylsulfonyl” is an alkyl radical attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” are NH₂O₂S—.

The phrase “therapeutically-effective” is intended to qualify the amount of each agent (i.e. the amount of cyclooxygenase-2 selective inhibitor and the amount of anticonvulsant) that will achieve the goal of improvement in severity of a CNS disorder or related condition and the frequency of incidence of the disorder or symptoms over no treatment, while avoiding adverse side effects typically associated with alternative therapies.

The term “treatment” includes alleviation, elimination of causation, or prevention of undesirable symptoms associated with CNS disorders or related conditions, such as seizures. Treatment as used herein includes prophylactic treatment.

The term “valproate” is used herein as a generic term referring to valproate or any of its related salts, esters, or derivatives, including for example, valproic acid, valproate sodium, divalproex, or divalproex sodium.

### DETAILED DESCRIPTION OF THE PREFERRED EMBODIMENTS

The present invention provides a combination therapy comprising the administration to a subject of a therapeutically effective amount of a COX-2 selective inhibitor in combination with a therapeutically effective amount of an anticonvulsant agent. The combination therapy may be used to treat a number of different CNS disorders or related conditions. In one embodiment, the combination therapy is used to treat or control seizures or seizure disorders in a subject. In one particular embodiment, the combination therapy is used to treat or control epilepsy in a subject. In another embodiment, the combination therapy is used to treat or control mood disorders, such as mania, depression, or bipolar disorder in a subject. In still another embodiment, the combination therapy is used to treat or control anxiety, anxiety disorders, sleep disorders, or agitation in a subject. In yet another embodiment, the combination therapy is used to treat or control a neurodegenerative or central nervous system related disorder in a subject. In a further embodiment, the combination therapy is used to treat a subject suffering from withdrawal from substances of abuse. When administered as part of a combination therapy, the COX-2 selective inhibitor together with the anticonvulsant agent provide enhanced treatment options as compared to administration of either the anticonvulsant agent or the COX-2 selective inhibitor alone.

Cyclooxygenase-2 Selective Inhibitors

A number of suitable cyclooxygenase-2 selective inhibitors or an isomer, a pharmaceutically acceptable salt, ester, or prodrug thereof, may be employed in the composition of the current invention. In one embodiment, the cyclooxygenase-2 selective inhibitor can be, for example, the cyclooxygenase-2 selective inhibitor meloxicam, Formula B-1 (CAS registry number 71125-38-7) or an isomer, a pharmaceutically acceptable salt, ester, or prodrug of a compound having Formula B-1.

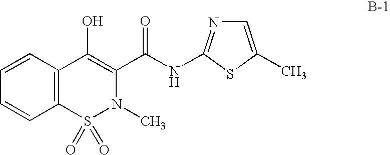

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or an isomer, a pharmaceutically acceptable salt, ester, or prodrug of a compound having Formula B-2.

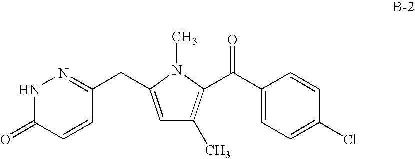

In still another embodiment the cyclooxygenase-2 selective inhibitor is a chromene compound that is a substituted benzopyran or a substituted benzopyran analog, and even more typically, selected from the group consisting of substituted benzothiopyrans, dihydroquinolines, dihydronaphthalenes or a compound having

Formula/shown below and possessing, by way of example and not limitation, the structures disclosed in Table 1. Furthermore, benzopyran cyclooxygenase-2 selective inhibitors useful in the practice of the present methods are described in U.S. Pat. Nos. 6,034,256 and 6,077,850 herein incorporated by reference in their entirety.

In another embodiment, the cyclooxygenase-2 selective inhibitor is a chromene compound represented by Formula/or an isomer, a pharmaceutically acceptable salt, ester, or prodrug thereof: 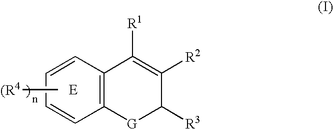
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof,
wherein:
n is an integer which is 0, 1, 2, 3 or 4;
G is O, S or NR^{a};
R¹ is H;
R^{a} is alkyl;

R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with the carbon atoms to which it is attached and the remainder of ring E forms a naphthyl radical.

In a further embodiment, the cyclooxygenase-2 selective inhibitor may also be a compound of Formula (I), or an isomer, a pharmaceutically acceptable salt, ester, or prodrug thereof,
wherein:
n is an integer which is 0, 1, 2, 3 or 4;
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof,
wherein:
R² is carboxyl;
R³ is lower haloalkyl; and
each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with the carbon atoms to which it is attached and the remainder of ring E forms a naphthyl radical. The cyclooxygenase-2 selective inhibitor may also be a compound of Formula (I) or an isomer, a pharmaceutically acceptable salt, ester, or prodrug thereof,
wherein:
n is an integer which is 0, 1, 2, 3 or 4;
R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl) aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof,
wherein:
n is an integer which is 0, 1, 2, 3 or 4;
R³ is trifluoromethyl or pentafluoroethyl; and
each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (I) or an isomer, a pharmaceutically acceptable salt, ester, or prodrug thereof,
wherein:
n is 4;
G is O or S;
R¹ is H;
R² is CO₂H;
R³ is lower haloalkyl;
a first R⁴ corresponding to R⁹ is hydrido or halo;
a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;
a third R⁴ corresponding to R¹¹ is H, lower alkyl, halo, lower alkoxy, or aryl; and
a fourth R⁴ corresponding to R¹² is H, halo, lower alkyl, lower alkoxy, or aryl;
wherein Formula (I) is represented by Formula (Ia):

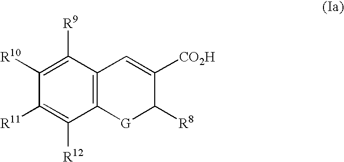

The cyclooxygenase-2 selective inhibitor used in connection with the method(s) of the present invention can also be a compound of having the structure of Formula (Ia) or an isomer, a pharmaceutically acceptable salt, ester, or prodrug thereof,
wherein:
G is O or S;
R⁸ is trifluoromethyl or pentafluoroethyl;
R⁹ is H, chloro, or fluoro;
R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;
R¹¹ is H, methyl, ethyl, isopropyl, tert-butyl, chloro, methoxy, diethylamino, or phenyl; and
R¹² is H, chloro, bromo, fluoro, methyl, ethyl, tert-butyl, methoxy, or phenyl.

Examples of exemplary chromene cyclooxygenase-2 selective inhibitors are depicted in Table 1 below.

TABLE 1
EXAMPLES OF CHROMENE CYCLOOXYGENASE-2 SELECTIVE
INHIBITORS AS EMBODIMENTS

| Compound | |
|---|---|
| Number | Structural Formula |
| B-3 | 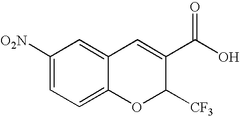 |
| | 6-Nitro-2-trifluoromethyl-2H-1- |
| | benzopyran-3-carboxylic acid |
| B-4 | 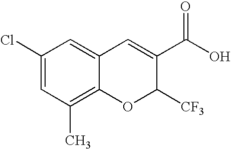 |
| | 6-Chioro-8-methyl-2-trifluoromethyl- |
| | 2H-1-benzopyran-3-carboxylic acid |
| B-5 | 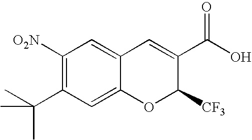 |
| | ((S)-6-Chloro-7-(1,1-dimethylethyl)-2-(trifluoro- |
| | methyl-2H-1-benzopyran-3-carboxylic acid |
| B-6 | 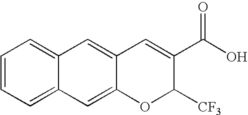 |
| | 2-Trifluoromethyl-2H-naphtho [2,3-b] |
| | pyran-3-carboxylic acid |
| B-7 | 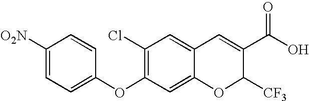 |
| | 6-Chloro-7-(4-nitrophenoxy)-2-(trifluoromethyl)-2H-1- |
| | benzopyran-3-carboxylic acid |
| B-8 | 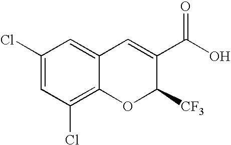 |
| | ((S)-6,8-Dichloro-2-(trifluoromethyl)- |
| | 2H-1-benzopyran-3-carboxylic acid |
| B-9 | 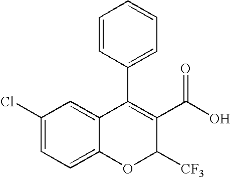 |
| | 6-Chloro-2-(trifluoromethyl)-4-phenyl-2H- |
| | 1-benzopyran-3-carboxylic acid |
| B-10 | 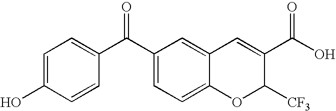 |
| | 6-(4-Hydroxybenzoyl)-2-(trifluoromethyl)- |
| | 2H-1-benzopyran-3-carboxylic acid |
| B-11 | 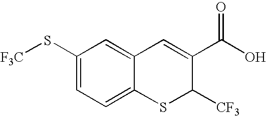 |
| | 2-(Trifluoromethyl)-6-[(trifluoromethyl)thio]- |
| | 2H-1-benzothiopyran-3-carboxylic acid |
| B-12 | 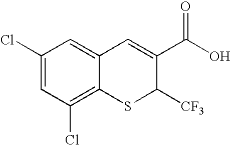 |
| | 6,8-Dichloro-2-trifluoromethyl-2H-1- |
| | benzothiopyran-3-carboxylic acid |
| B-13 | 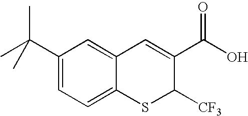 |
| | 6-(1,1-Dimethylethyl)-2-(trifluoromethyl)- |
| | 2H-1-benzothiopyran-3-carboxylic acid |
| B-14 | 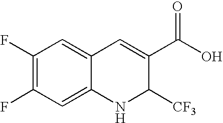 |
| | 6,7-Difluoro-1,2-dihydro-2-(trifluoro- |
| | methyl)-3-quinolinecarboxylic acid |
| B-15 | 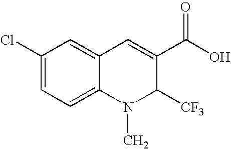 |
| | 6-Chloro-1,2-dihydro-1-methyl-2-(trifluoro- |
| | methyl)-3-quinolinecarboxylic acid |
| B-16 | 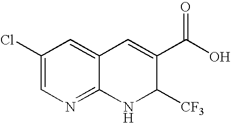 |
| | 6-Chloro-2-(trifluoromethyl)-1,2-dihydro |
| | [1,8]naphthyridine-3-carboxylic acid |
| B-17 | 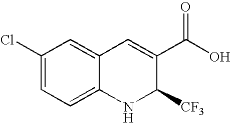 |
| | ((S)-6-Chloro-1,2-dihydro-2-(trifluoro- |
| | methyl)-3-quinolinecarboxylic acid |

In a further embodiment, the cyclooxygenase-2 selective inhibitor is selected from the class of tricyclic cyclooxygenase-2 selective inhibitors represented by the general structure of Formula II or an isomer, a pharmaceutically acceptable salt, ester, or prodrug thereof, 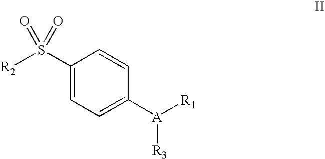
wherein:
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and

R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

In another embodiment, the cyclooxygenase-2 selective inhibitor represented by the above Formula II is selected from the group of compounds illustrated in Table 2, consisting of celecoxib (B-18; U.S. Pat. No. 5,466,823; CAS No. 169590-42-5), valdecoxib (B-19; U.S. Pat. No. 5,633,272; CAS No. 181695-72-7), deracoxib (B-20; U.S. Pat. No. 5,521,207; CAS No. 169590-41-4), rofecoxib (B-21; CAS No. 162011-90-7), etoricoxib (MK-663; B-22; PCT publication WO 98/03484), tilmacoxib (JTE-522; B-23; CAS No. 180200-68-4).

TABLE 2
EXAMPLES OF TRICYCLIC CYCLOOXYGENASE-2 SELECTIVE
INHIBITORS AS EMBODIMENTS

| Compound | |
|---|---|
| Number | Structural Formula |
| B-18 | 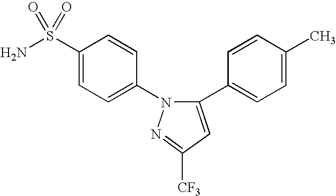 |
| B-19 | 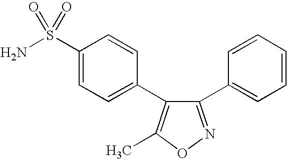 |
| B-20 | 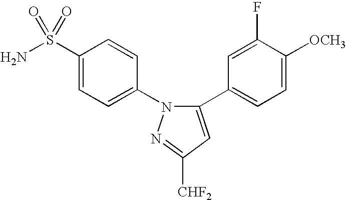 |
| B-21 | 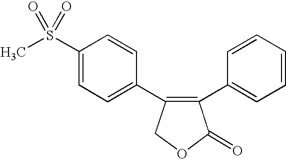 |
| B-22 | 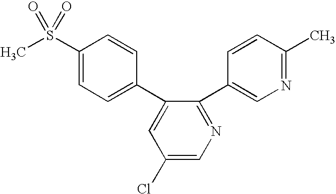 |
| B-23 | 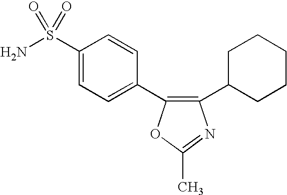 |

In still another embodiment, the cyclooxygenase-2 selective inhibitor is selected from the group consisting of celecoxib, rofecoxib and etoricoxib.

In yet another embodiment, the cyclooxygenase-2 selective inhibitor is parecoxib (B-24, U.S. Pat. No. 5,932,598, CAS No. 198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

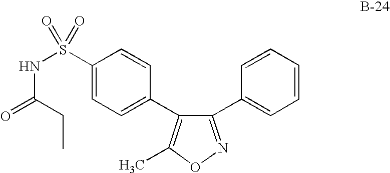

One form of parecoxib is sodium parecoxib.

In another embodiment of the invention, the compound having the formula B-25 or an isomer, a pharmaceutically acceptable salt, ester, or prodrug of a compound having formula B-25 that has been previously described in International Publication number WO 00/24719 (which is herein incorporated by reference) is another tricyclic cyclooxygenase-2 selective inhibitor that may be advantageously employed.

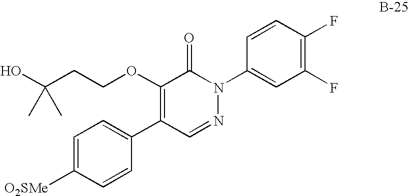

Another cyclooxygenase-2 selective inhibitor that is useful in connection with the method(s) of the present invention is N-(2-cyclohexyloxynitrophenyl)-methane sulfonamide (NS-398) having a structure shown below as B-26, or an isomer, a pharmaceutically acceptable salt, ester, or prodrug of a compound having formula B-26.

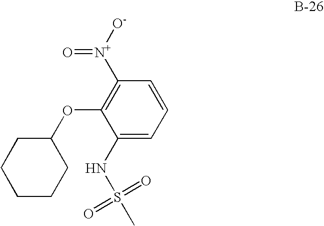

In yet a further embodiment, the cyclooxygenase-2 selective inhibitor used in connection with the method(s) of the present invention can be selected from the class of phenylacetic acid derivative cyclooxygenase-2 selective inhibitors represented by the general structure of Formula (III) or an isomer, a pharmaceutically acceptable salt, ester, or prodrug thereof: 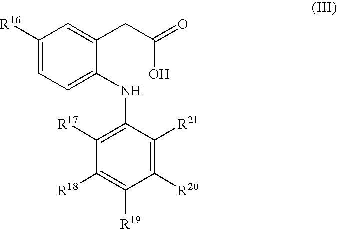
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl, provided, however, that each of R¹⁷, R¹⁸, R¹⁹ and R²⁰ is not fluoro when R¹⁶ is ethyl and R¹⁹ is H.

Another phenylacetic acid derivative cyclooxygenase-2 selective inhibitor used in connection with the method(s) of the present invention is a compound that has the designation of COX 189 (lumiracoxib; B-211) and that has the structure shown in Formula (III) or an isomer, a pharmaceutically acceptable salt, ester, or prodrug thereof wherein:
R¹⁶ is ethyl;
R¹⁷ and R¹⁹ are chloro;
R¹⁸ and R²⁰ are hydrogen; and
R²¹ is methyl.

In yet another embodiment, the cyclooxygenase-2 selective inhibitor is represented by Formula (IV) or an isomer, a pharmaceutically acceptable salt, ester, or prodrug thereof: 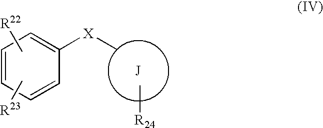
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

According to another embodiment, the cyclooxygenase-2 selective inhibitors used in the present method(s) have the structural Formula (V) or an isomer, a pharmaceutically acceptable salt, ester, or prodrug thereof: 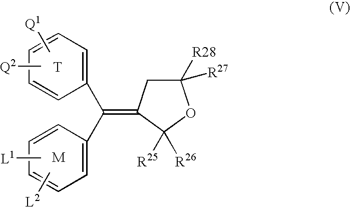
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and
at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an
—SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or,
R²⁷ and R²⁸ are O; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

In another embodiment, the compounds N-(2-cyclohexyloxynitrophenyl) methane sulfonamide, and (E)-4-[(4-methylphenyl)(tetrahydro-2-oxo-3-furanylidene) methyl]benzenesulfonamide or an isomer, a pharmaceutically acceptable salt, ester, or prodrug thereof having the structure of Formula (V) are employed as cyclooxygenase-2 selective inhibitors.

In a further embodiment, compounds that are useful for the cyclooxygenase-2 selective inhibitor or an isomer, a pharmaceutically acceptable salt, ester, or prodrug thereof used in connection with the method(s) of the present invention, the structures for which are set forth in Table 3 below, include, but are not limited to:
6-chloro-2-trifluoromethyl-2H-1-benzopyran-3-carboxylic acid (B-27);
6-chloro-7-methyl-2-trifluoromethyl-2H-1-benzopyran-3-carboxylic acid (B-28);
8-(1-methylethyl)-2-trifluoromethyl-2H-1-benzopyran-3-carboxylic acid (B-29);
6-chloro-8-(1-methylethyl)-2-trifluoromethyl-2H-1-benzopyran-3-carboxylic acid (B-30);
2-trifluoromethyl-3H-naphtho[2,1-b]pyran-3-carboxylic acid (B-31);
7-(1,1-dimethylethyl)-2-trifluoromethyl-2H-1-benzopyran-3-carboxylic acid (B-32);
6-bromo-2-trifluoromethyl-2H-1-benzopyran-3-carboxylic acid (B-33);
8-chloro-2-trifluoromethyl-2H-1-benzopyran-3-carboxylic acid (B-34);
6-trifluoromethoxy-2-trifluoromethyl-2H-1-benzopyran-3-carboxylic acid (B-35);
5,7-dichloro-2-trifluoromethyl-2H-1-benzopyran-3-carboxylic acid (B-36);
8-phenyl-2-trifluoromethyl-2H-1-benzopyran-3-carboxylic acid (B-37);
7,8-dimethyl-2-trifluoromethyl-2H-1-benzopyran-3-carboxylic acid (B-38);
6,8-bis(dimethylethyl)-2-trifluoromethyl-2H-1-benzopyran-3-carboxylic acid (B-39);
7-(1-methylethyl)-2-trifluoromethyl-2H-1-benzopyran-3-carboxylic acid (B-40);
7-phenyl-2-trifluoromethyl-2H-1-benzopyran-3-carboxylic acid (B-41);
6-chloro-7-ethyl-2-trifluoromethyl-2H-1-benzopyran-3-carboxylic acid (B-42);
6-chloro-8-ethyl-2-trifluoromethyl-2H-1-benzopyran-3-carboxylic acid (B-43);
6-chloro-7-phenyl-2-trifluoromethyl-2H-1-benzopyran-3-carboxylic acid (B-44);
6,7-dichloro-2-trifluoromethyl-2H-1-benzopyran-3-carboxylic acid (B-45);
6,8-dichloro-2-trifluoromethyl-2H-1-benzopyran-3-carboxylic acid (B-46);
6-chloro-8-methyl-2-trifluoromethyl-2H-1-benzopyran-3-carboxylic acid (B-47);
8-chloro-6-methyl-2-trifluoromethyl-2H-1-benzopyran-3-carboxylic acid (B-48)
8-chloro-6-methoxy-2-trifluoromethyl-2H-1-benzopyran-3-carboxylic acid (B-49);
6-bromo-8-chloro-2-trifluoromethyl-2H-1-benzopyran-3-carboxylic acid (B-50);
8-bromo-6-fluoro-2-trifluoromethyl-2H-1-benzopyran-3-carboxylic acid (B-51);
8-bromo-6-methyl-2-trifluoromethyl-2H-1-benzopyran-3-carboxylic acid (B-52);
8-bromo-5-fluoro-2-trifluoromethyl-2H-1-benzopyran-3-carboxylic acid (B-53);
6-chloro-8-fluoro-2-trifluoromethyl-2H-1-benzopyran-3-carboxylic acid (B-54);
6-bromo-8-methoxy-2-trifluoromethyl-2H-1-benzopyran-3-carboxylic acid (B-55);
6-[[(phenylmethyl)amino]sulfonyl]-2-trifluoromethyl-2H-1-benzopyran-3-carboxylic acid (B-56);
6-[(dimethylamino)sulfonyl]-2-trifluoromethyl-2H-1-benzopyran-3-carboxylic acid (B-57);
6-[(methylamino)sulfonyl]-2-trifluoromethyl-2H-1-benzopyran-3-carboxylic acid (B-58);
6-[(4-morpholino)sulfonyl]-2-trifluoromethyl-2H-1-benzopyran-3-carboxylic acid (B-59);
6-[(1,1-dimethylethyl)aminosulfonyl]-2-trifluoromethyl-2H-1-benzopyran-3-carboxylic acid (B-60);
6-[(2-methylpropyl)aminosulfonyl]-2-trifluoromethyl-2H-1-benzopyran-3-carboxylic acid (B-61);
6-methylsulfonyl-2-trifluoromethyl-2H-1-benzopyran-3-carboxylic acid (B-62);
8-chloro-6-[[(phenylmethyl)amino]sulfonyl]-2-trifluoromethyl-2H-1-benzopyran-3-carboxylic acid (B-63);
6-phenylacetyl-2-trifluoromethyl-2H-1-benzopyran-3-carboxylic acid (B-64);
6,8-dibromo-2-trifluoromethyl-2H-1-benzopyran-3-carboxylic acid (B-65);
8-chloro-5,6-dimethyl-2-trifluoromethyl-2H-1-benzopyran-3-carboxylic acid (B-66);
6,8-dichloro-(S)-2-trifluoromethyl-2H-1-benzopyran-3-carboxylic acid (B-67);
6-benzylsulfonyl-2-trifluoromethyl-2H-1-benzopyran-3-carboxylic acid (B-68);
6-[[N-(2-furylmethyl)amino]sulfonyl]-2-trifluoromethyl-2H-1-benzopyran-3-carboxylic acid (B-69);
6-[[N-(2-phenylethyl)amino]sulfonyl]-2-trifluoromethyl-2H-1-benzopyran-3-carboxylic acid (B-70);
6-iodo-2-trifluoromethyl-2H-1-benzopyran-3-carboxylic acid (B-71);
7-(1,1-dimethylethyl)-2-pentafluoroethyl-2H-1-benzopyran-3-carboxylic acid (B-72);
6-chloro-2-trifluoromethyl-2H-1-benzothiopyran-3-carboxylic acid (B-73);
3-[(3-chloro-phenyl)-(4-methanesulfonyl-phenyl)-methylene]-dihydro-furan-2-one or BMS-347070 (B-74);
8-acetyl-3-(4-fluorophenyl)-2-(4-methylsulfonyl)phenyl-imidazo(1,2-a) pyridine (B-75);
5,5-dimethyl-4-(4-methylsulfonyl)phenyl-3-phenyl-2-(5H)-furanone (B-76);
5-(4-fluorophenyl)-1-[4-(methylsulfonyl)phenyl]-3-(trifluoromethyl)pyrazole (B-77);
4-(4-fluorophenyl)-5-[4-(methylsulfonyl)phenyl]-1-phenyl-3-(trifluoromethyl)pyrazole (B-78);
4-(5-(4-chlorophenyl)-3-(4-methoxyphenyl)-1H-pyrazol-1-yl) benzenesulfonamide (B-79);
4-(3,5-bis(4-methylphenyl)-1H-pyrazol-1-yl) benzenesulfonamide (B-80);
4-(5-(4-chlorophenyl)-3-phenyl-1H-pyrazol-1-yl) benzenesulfonamide (B-81);
4-(3,5-bis(4-methoxyphenyl)-1H-pyrazol-1-yl) benzenesulfonamide (B-82);
4-(5-(4-chlorophenyl)-3-(4-methylphenyl)-1H-pyrazol-1-yl) benzenesulfonamide (B-83);
4-(5-(4-chlorophenyl)-3-(4-nitrophenyl)-1H-pyrazol-1-yl) benzenesulfonamide (B-84);
4-(5-(4-chlorophenyl)-3-(5-chloro-2-thienyl)-1H-pyrazol-1-yl) benzenesulfonamide (B-85);
4-(4-chloro-3,5-diphenyl-1H-pyrazol-1-yl)benzenesulfonamide (B-86);
4-[5-(4-chlorophenyl)-3-(trifluoromethyl)-1H-pyrazol-1-yl] benzenesulfonamide (B-87);
4-[5-phenyl-3-(trifluoromethyl)-1H-pyrazol-1-yl] benzenesulfonamide (B-88);
4-[5-(4-fluorophenyl)-3-(trifluoromethyl)-1H-pyrazol-1-yl] benzenesulfonamide (B-89);
4-[5-(4-methoxyphenyl)-3-(trifluoromethyl)-1H-pyrazol-1-yl] benzenesulfonamide (B-90);
4-[5-(4-chlorophenyl)-3-(difluoromethyl)-1H-pyrazol-1-yl] benzenesulfonamide (B-91);
4-[5-(4-methylphenyl)-3-(trifluoromethyl)-1H-pyrazol-1-yl] benzenesulfonamide (B-92);
4-[4-chloro-5-(4-chlorophenyl)-3-(trifluoromethyl)-1H-pyrazol-1-yl]benzenesulfonamide (B-93);
4-[3-(difluoromethyl)-5-(4-methylphenyl)-1H-pyrazol-1-yl] benzenesulfonamide (B-94);
4-[3-(difluoromethyl)-5-phenyl-1H-pyrazol-1-yl] benzenesulfonamide (B-95);
4-[3-(difluoromethyl)-5-(4-methoxyphenyl)-1H-pyrazol-1-yl] benzenesulfonamide (B-96);
4-[3-cyano-5-(4-fluorophenyl)-1H-pyrazol-1-yl] benzenesulfonamide (B-97);
4-[3-(difluoromethyl)-5-(3-fluoro-4-methoxyphenyl)-1H-pyrazol-1-yl] benzenesulfonamide (B-98);
4-[5-(3-fluoro-4-methoxyphenyl)-3-(trifluoromethyl)-1H-pyrazol-1-yl] benzenesulfonamide (B-99);
4-[4-chloro-5-phenyl-1H-pyrazol-1-yl]benzenesulfonamide (B-100);
4-[5-(4-chlorophenyl)-3-(hydroxymethyl)-1H-pyrazol-1-yl] benzenesulfonamide (B-101);
4-[5-(4-(N,N-dimethylamino)phenyl)-3-(trifluoromethyl)-1H-pyrazol-1-yl] benzenesulfonamide (B-102);
5-(4-fluorophenyl)-6-[4-(methylsulfonyl)phenyl]spiro[2.4]hept-5-ene (B-103);
4-[6-(4-fluorophenyl)spiro[2.4]hept-5-en-5-yl] benzenesulfonamide (B-104);
6-(4-fluorophenyl)-7-[4-(methylsulfonyl)phenyl]spiro[3.4]oct-6-ene (B-105);
5-(3-chloro-4-methoxyphenyl)-6-[4-(methylsulfonyl) phenyl]spiro [2.4]hept-5-ene (B-106);
4-[6-(3-chloro-4-methoxyphenyl)spiro[2.4]hept-5-en-5-yl] benzenesulfonamide (B-107);
5-(3,5-dichloro-4-methoxyphenyl)-6-[4-(methylsulfonyl)phenyl]spiro [2.4]hept-5-ene (B-108);
5-(3-chloro-4-fluorophenyl)-6-[4-(methylsulfonyl)phenyl] spiro[2.4]hept-5-ene (B-109);
4-[6-(3,4-dichlorophenyl)spiro[2.4]hept-5-en-5-yl] benzenesulfonamide (B-110);
2-(3-chloro-4-fluorophenyl)-4-(4-fluorophenyl)-5-(4-methylsulfonyl phenyl)thiazole (B-111);
2-(2-chlorophenyl)-4-(4-fluorophenyl)-5-(4-methylsulfonyl phenyl)thiazole (B-112);
5-(4-fluorophenyl)-4-(4-methylsulfonylphenyl)-2-methylthiazole (B-113);
4-(4-fluorophenyl)-5-(4-methylsulfonylphenyl)-2-trifluoromethylthiazole (B-114);
4-(4-fluorophenyl)-5-(4-methylsulfonylphenyl)-2-(2-thienyl)thiazole (B-115);
4-(4-fluorophenyl)-5-(4-methylsulfonylphenyl)-2-benzylaminothiazole (B-116);
4-(4-fluorophenyl)-5-(4-methylsulfonylphenyl)-2-(1-propylamino) thiazole (B-117);
2-[(3,5-dichlorophenoxy)methyl)-4-(4-fluorophenyl)-5-[4-(methyl sulfonyl)phenyl]thiazole (B-118);
5-(4-fluorophenyl)-4-(4-methylsulfonylphenyl)-2-trifluoromethylthiazole (B-119);
1-methylsulfonyl-4-[1,1-dimethyl-4-(4-fluorophenyl)cyclopenta-2,4-dien-3-yl]benzene (B-120);
4-[4-(4-fluorophenyl)-1,1-dimethylcyclopenta-2,4-dien-3-yl] benzenesulfonamide (B-121);
5-(4-fluorophenyl)-6-[4-(methylsulfonyl)phenyl]spiro[2.4]hepta-4,6-diene (B-122);
4-[6-(4-fluorophenyl)spiro[2.4]hepta-4,6-dien-5-yl] benzenesulfonamide (B-123);
6-(4-fluorophenyl)-2-methoxy-5-[4-(methylsulfonyl)phenyl]-pyridine-3-carbonitrile (B-124);
2-bromo-6-(4-fluorophenyl)-5-[4-(methylsulfonyl)phenyl]-pyridine-3-carbonitrile (B-125);
6-(4-fluorophenyl)-5-[4-(methylsulfonyl)phenyl]-2-phenyl-pyrid ine-3-carbonitrile (B-126);
4-[2-(4-methylpyridin-2-yl)-4-(trifluoromethyl)-1H-imidazol-1-yl] benzenesulfonamide (B-127);
4-[2-(5-methylpyridin-3-yl)-4-(trifluoromethyl)-1H-imidazol-1-yl] benzenesulfonamide (B-128);
4-[2-(2-methylpyridin-3-yl)-4-(trifluoromethyl)-1H-imidazol-1-yl] benzenesulfonamide (B-129);
3-[1-[4-(methylsulfonyl)phenyl]-4-(trifluoromethyl)-1H-imidazol-2-yl] pyridine (B-130);
2-[1-[4-(methylsulfonyl)phenyl-4-(trifluoromethyl)-1H-imidazol-2-yl]pyridine (B-131);
2-methyl-4-[1-[4-(methylsulfonyl)phenyl-4-(trifluoromethyl)-1H-imidazol-2-yl]pyridine (B-132);
2-methyl-6-[1-[4-(methylsulfonyl)phenyl-4-(trifluoromethyl)-1H-imidazol-2-yl]pyridine (B-133);
4-[2-(6-methylpyridin-3-yl)-4-(trifluoromethyl)-1H-imidazol-1-yl] benzenesulfonamide (B-134);
2-(3,4-difluorophenyl)-1-[4-(methylsulfonyl)phenyl]-4-(trifluoromethyl)-1H-imidazole (B-135);
4-[2-(4-methylphenyl)-4-(trifluoromethyl)-1H-imidazol-1-yl] benzenesulfonamide (B-136);
2-(4-chlorophenyl)-1-[4-(methylsulfonyl)phenyl]-4-methyl-1H-imidazole (B-137);
2-(4-chlorophenyl)-1-[4-(methylsulfonyl)phenyl]-4-phenyl-1H-imidazole (B-138);
2-(4-chlorophenyl)-4-(4-fluorophenyl)-1-[4-(methylsulfonyl)phenyl]-1H-imidazole (B-139);
2-(3-fluoro-4-methoxyphenyl)-1-[4-(methylsulfonyl)phenyl-4-(trifluoro methyl)-1H-imidazole (B-140);
1-[4-(methylsulfonyl)phenyl]-2-phenyl-4-trifluoromethyl-1H-imidazole (B-141);
2-(4-methylphenyl)-1-[4-(methylsulfonyl)phenyl]-4-trifluoromethyl-1H-imidazole (B-142);
4-[2-(3-chloro-4-methylphenyl)-4-(trifluoromethyl)-1H-imidazol-1-yl] benzenesulfonamide (B-143);
2-(3-fluoro-5-methylphenyl)-1-[4-(methylsulfonyl)phenyl]-4-(trifluoro methyl)-1H-imidazole (B-144);
4-[2-(3-fluoro-5-methylphenyl)-4-(trifluoromethyl)-1H-imidazol-1-yl] benzenesulfonamide (B-145);
2-(3-methylphenyl)-1-[4-(methylsulfonyl)phenyl]-4-trifluoromethyl-1H-imidazole (B-146);
4-[2-(3-methylphenyl)-4-trifluoromethyl-1H-imidazol-1-yl]benzene sulfonamide (B-147);
1-[4-(methylsulfonyl)phenyl]-2-(3-chlorophenyl)-4-trifluoromethyl-1H-imidazole (B-148);
4-[2-(3-chlorophenyl)-4-trifluoromethyl-1H-imidazol-1-yl] benzenesulfonamide (B-149);
4-[2-phenyl-4-trifluoromethyl-1H-imidazol-1-yl] benzenesulfonamide (B-150);
4-[2-(4-methoxy-3-chlorophenyl)-4-trifluoromethyl-1H-imidazol-1-yl] benzenesulfonamide (B-151);
1-allyl-4-(4-fluorophenyl)-3-[4-(methylsulfonyl)phenyl]-5-(trifluoro methyl)-1H-pyrazole (B-152);
4-[1-ethyl-4-(4-fluorophenyl)-5-(trifluoromethyl)-1H-pyrazol-3-yl] benzenesulfonamide (B-153);
N-phenyl-[4-(4-fluorophenyl)-3-[4-(methylsulfonyl)phenyl]-5-(trifluoromethyl)-1H-pyrazol-1-yl]acetamide (B-154);
ethyl [4-(4-fluorophenyl)-3-[4-(methylsulfonyl)phenyl]-5-(trifluoromethyl)-1H-pyrazol-1-yl]acetate (B-155);
4-(4-fluorophenyl)-3-[4-(methylsulfonyl)phenyl]-1-(2-phenylethyl)-1H-pyrazole (B-156);
4-(4-fluorophenyl)-3-[4-(methylsulfonyl)phenyl]-1-(2-phenylethyl)-5-(trifluoromethyl)pyrazole (B-157);
1-ethyl-4-(4-fluorophenyl)-3-[4-(methylsulfonyl)phenyl]-5-(trifluoromethyl)-1H-pyrazole (B-158);
5-(4-fluorophenyl)-4-(4-methylsulfonylphenyl)-2-trifluoromethyl-1H-imidazole (B-159);
4-[4-(methylsulfonyl)phenyl]-5-(2-thiophenyl)-2-(trifluoromethyl)-1H-imidazole (B-160);
5-(4-fluorophenyl)-2-methoxy-4-[4-(methylsulfonyl)phenyl]-6-(trifluoromethyl)pyridine (B-161);
2-ethoxy-5-(4-fluorophenyl)-4-[4-(methylsulfonyl)phenyl]-6-(trifluoromethyl)pyridine (B-162);
5-(4-fluorophenyl)-4-[4-(methylsulfonyl)phenyl]-2-(2-propynyloxy)-6-(trifluoromethyl)pyridine (B-163);
2-bromo-5-(4-fluorophenyl)-4-[4-(methylsulfonyl)phenyl]-6-(trifluoromethyl)pyridine (B-164);
4-[2-(3-chloro-4-methoxyphenyl)-4,5-difluorophenyl]benzenesulfonamide (B-165);
1-(4-fluorophenyl)-2-[4-(methylsulfonyl)phenyl]benzene (B-166);
5-difluoromethyl-4-(4-methylsulfonylphenyl)-3-phenylisoxazole (B-167);
4-[3-ethyl-5-phenylisoxazol-4-yl]benzenesulfonamide (B-168);
4-[5-difluoromethyl-3-phenylisoxazol-4-yl]benzenesulfonamide (B-169);
4-[5-hydroxymethyl-3-phenylisoxazol-4-yl]benzenesulfonamide (B-170);
4-[5-methyl-3-phenyl-isoxazol-4-yl]benzenesulfonamide (B-171);
1-[2-(4-fluorophenyl)cyclopenten-1-yl]-4-(methylsulfonyl) benzene (B-172);
1-[2-(4-fluoro-2-methylphenyl)cyclopenten-1-yl]-4-(methylsulfonyl) benzene (B-173);
1-[2-(4-chlorophenyl)cyclopenten-1-yl]-4-(methylsulfonyl) benzene (B-174);
1-[2-(2,4-dichlorophenyl)cyclopenten-1-yl]-4-(methylsulfonyl) benzene (B-175);
1-[2-(4-trifluoromethylphenyl)cyclopenten-1-yl]-4-(methylsulfonyl) benzene (B-176);
1-[2-(4-methylthiophenyl)cyclopenten-1-yl]-4-(methyl sulfonyl)benzene (B-177);
1-[2-(4-fluorophenyl)-4,4-dimethylcyclopenten-1-yl]-4-(methylsulfonyl) benzene (B-178);
4-[2-(4-fluorophenyl)-4,4-dimethylcyclopenten-1-yl]benzene sulfonamide (B-179);
1-[2-(4-chlorophenyl)-4,4-dimethylcyclopenten-1-yl]-4-(methylsulfonyl) benzene (B-180);
4-[2-(4-chlorophenyl)-4,4-dimethylcyclopenten-1-yl]benzene sulfonamide (B-181);
4-[2-(4-fluorophenyl)cyclopenten-1-yl]benzenesulfonamide (B-182);
4-[2-(4-chlorophenyl)cyclopenten-1-yl]benzenesulfonamide (B-183);
1-[2-(4-methoxyphenyl)cyclopenten-1-yl]-4-(methylsulfonyl) benzene (B-184);
1-[2-(2,3-difluorophenyl)cyclopenten-1-yl]-4-(methylsulfonyl) benzene (B-185);
4-[2-(3-fluoro-4-methoxyphenyl)cyclopenten-1-yl] benzenesulfonamide (B-186);
1-[2-(3-chloro-4-methoxyphenyl)cyclopenten-1-yl]-4-(methylsulfonyl) benzene (B-187);
4-[2-(3-chloro-4-fluorophenyl)cyclopenten-1-yl] benzenesulfonamide (B-188);
4-[2-(2-methylpyridin-5-yl)cyclopenten-1-yl]benzenesulfonamide (B-189);
ethyl 2-[4-(4-fluorophenyl)-5-[4-(methylsulfonyl) phenyl]oxazol-2-yl]-2-benzyl-acetate (B-190);
2-[4-(4-fluorophenyl)-5-[4-(methylsulfonyl)phenyl]oxazol-2-yl] acetic acid (B-191);
2-(tert-butyl)-4-(4-fluorophenyl)-5-[4-(methylsulfonyl)phenyl] oxazole (B-192);
4-(4-fluorophenyl)-5-[4-(methylsulfonyl)phenyl]-2-phenyloxazole (B-193);
4-(4-fluorophenyl)-2-methyl-5-[4-(methylsulfonyl)phenyl]oxazole (B-194);
4-[5-(3-fluoro-4-methoxyphenyl)-2-trifluoromethyl-4-oxazolyl] benzenesulfonamide (B-195);
6-chloro-7-(1,1-dimethylethyl)-2-trifluoromethyl-2H-1-benzopyran-3-carboxylic acid (B-196);
6-chloro-8-methyl-2-trifluoromethyl-2H-1-benzopyran-3-carboxylic acid (B-197);
5,5-dimethyl-3-(3-fluorophenyl)-4-methylsulfonyl-2(5H)-furanone (B-198);
6-chloro-2-trifluoromethyl-2H-1-benzothiopyran-3-carboxylic acid (B-199);
4-[5-(4-chlorophenyl)-3-(trifluoromethyl)-1H-pyrazol-1-yl]benzene sulfonamide (B-200);
4-[5-(4-methylphenyl)-3-(trifluoromethyl)-1H-pyrazol-1-yl]benzene sulfonamide (B-201);
4-[5-(3-fluoro-4-methoxyphenyl)-3-(difluoromethyl)-1H-pyrazol-1-yl] benzenesulfonamide (B-202);
3-[1-[4-(methylsulfonyl)phenyl]-4-trifluoromethyl-1H-imidazol-2-yl] pyridine (B-203);
2-methyl-5-[1-[4-(methylsulfonyl)phenyl]-4-trifluoromethyl-1H-imidazol-2-yl]pyridine (B-204);
4-[2-(5-methylpyridin-3-yl)-4-(trifluoromethyl)-1H-imidazol-1-yl] benzenesulfonamide (B-205);
4-[5-methyl-3-phenylisoxazol-4-yl]benzenesulfonamide (B-206);
4-[5-hydroxymethyl-3-phenylisoxazol-4-yl]benzenesulfonamide (B-207);
[2-trifluoromethyl-5-(3,4-difluorophenyl)-4-oxazolyl] benzenesulfonamide (B-208);
4-[2-methyl-4-phenyl-5-oxazolyl]benzenesulfonamide (B-209);
4-[5-(2-fluoro-4-methoxyphenyl)-2-trifluoromethyl-4-oxazolyl] benzenesulfonamide (B-210);
[2-(2-chloro-6-fluoro-phenylamino)-5-methyl-phenyl]-acetic acid or COX 189 (lumiracoxib; B-211);
N-(4-Nitro-2-phenoxy-phenyl)-methanesulfonamide or nimesulide (B-212);
N-[6-(2,4-difluoro-phenoxy)-1-oxo-indan-5-yl]-methanesulfonamide or flosulide (B-213);
N-[6-(2,4-Difluoro-phenylsulfanyl)-1-oxo-1H-inden-5-yl]-methanesulfonamide, sodium salt or L-745337 (B-214);
N-[5-(4-fluoro-phenylsulfanyl)-thiophen-2-yl]-methanesulfonamide or RWJ-63556 (B-215);
3-(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216);
(5Z)-2-amino-5-[[3,5-bis(1,1-dimethylethyl)-4-hydroxyphenyl]methylene]-4(5H)-thiazolone or darbufelone (B-217);
CS-502 (B-218);
LAS-34475 (B-219);
LAS-34555 (B-220);
S-33516 (B-221);
SD-8381 (B-222);
L-783003 (B-223);
N-[3-(formylamino)-4-oxo-6-phenoxy-4H-1-benzopyran-7-yl]-methanesulfonamide or T-614 (B-224);
D-1367 (B-225);
L-748731 (B-226);
(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);
CGP-28238 (B-228);
4-[[3,5-bis(1,1-dimethylethyl)-4-hydroxyphenyl]methylene]dihydro-2-methyl-2H-1,2-oxazin-3(4H)-one or BF-389 (B-229);
GR-253035 (B-230);
6-dioxo-9H-purin-8-yl-cinnamic acid (B-231);
S-2474 (B-232);
4-[4-(methyl)-sulfonyl)phenyl]-3-phenyl-2(5H)-furanone;
4-(5-methyl-3-phenyl-4-isoxazolyl);
2-(6-methylpyrid-3-yl)-3-(4-methylsulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
4-[5-(3-fluoro-4-methoxyphenyl)-3-difluoromethyl)-1H-pyrazol-1-yl] benzenesulfonamide;
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methyl sulfonyl)phenyl]-3(2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid.

TABLE 3
EXAMPLES OF CYCLOOXYGENASE-2 SELECTIVE
INHIBITORS AS EMBODIMENTS

| Compound | |
|---|---|
| Number | Structural Formula |
| B-26 | 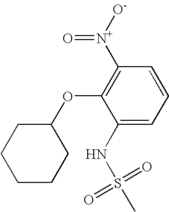 |
| | N-(2-cyclohexyloxynitrophenyl) methane |
| | sulfonamide or NS-398; |
| B-27 | 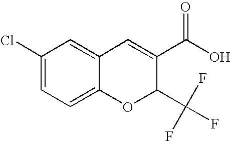 |
| | 6-chloro-2-tnfluoromethyl-2H-1-benzopyran-3- |
| | carboxylic acid; |
| B-28 | 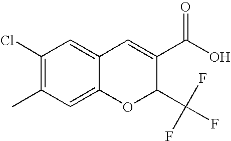 |
| | 6-chloro-7-methyl-2-trifluoromethyl-2H-1- |
| | benzopyran-3-carboxylic acid; |
| B-29 | 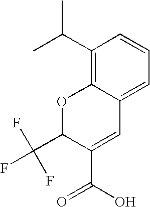 |
| | 8-(1-methylethyl)-2-trifluoromethyl-2H-1- |
| | benzopyran-3-carboxylic acid; |
| B-30 | 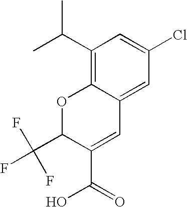 |
| | 6-chloro-8-(1-methylethyl)-2-trifluoromethyl- |
| | 2H-1-benzopyran-3-carboxylic acid; |
| B-31 | 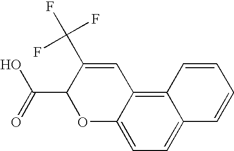 |
| | 2-trifluoromethyl-3H-naphtho[2,1-b]pyran-3- |
| | carboxylic acid; |
| B-32 | 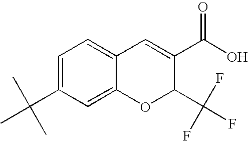 |
| | 7-(1,1-dimethylethyl)-2-trifluoromethyl-2H-1- |
| | benzopyran-3-carboxylic acid; |
| B-33 | 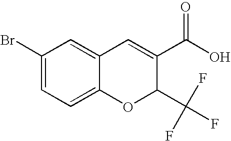 |
| | 6-bromo-2-trifluoromethyl-2H-1-benzopyran-3- |
| | carboxylic acid; |
| B-34 | 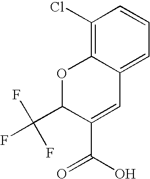 |
| | 8-chloro-2-trifluoromethyl-2H-1-benzopyran- |
| | 3-carboxylic acid; |
| B-35 | 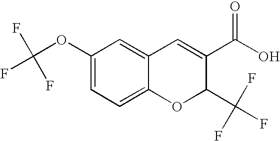 |
| | 6-trifluoromethoxy-2-trifluoromethyl-2H-1- |
| | benzopyran-3-carboxylic acid; |
| B-36 | 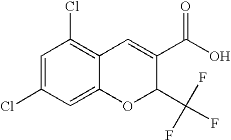 |
| | 5,7-dichloro-2-trifluoromethyl-2H-1-benzopyran-3- |
| | carboxylic acid; |
| B-37 | 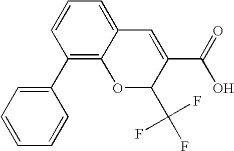 |
| | 8-phenyl-2-trifluoromethyl-2H-1-benzopyran-3- |
| | carboxylic acid; |
| B-38 | 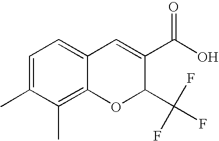 |
| | 7,8-dimethyl-2-trifluoromethyl-2H-1-benzopyran-3- |
| | carboxylic acid; |
| B-39 | 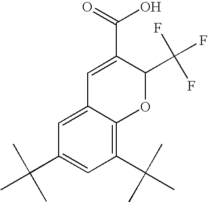 |
| | 6,8-bis(dimethylethyl)-2-trifluoromethyl-2H-1- |
| | benzopyran-3-carboxylic acid; |
| B-40 | 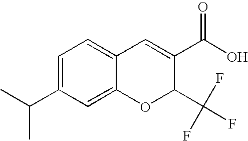 |
| | 7-(1-methylethyl)-2-trifluoromethyi-2H-1- |
| | benzopyran-3-carboxylic acid; |
| B-41 | 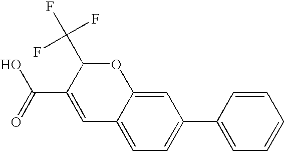 |
| | 7-phenyl-2-trifluoromethyl-2H-1-benzopyran-3- |
| | carboxylic acid; |
| B-42 | 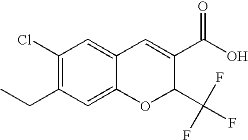 |
| | 6-chloro-7-ethyl-2-trifluoromethyl-2H-1-benzopyran- |
| | 3-carboxylic acid; |
| B-43 | 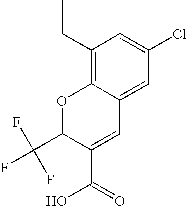 |
| | 6-chloro-8-ethyl-2-trifluoromethyl-2H-1-benzopyran-3- |
| | carboxylic acid; |
| B-44 | 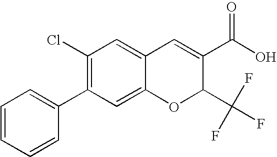 |
| | 6-chloro-7-phenyl-2-trifluoromethyl-2H-1-benzopyran- |
| | 3-carboxylic acid; |
| B-45 | 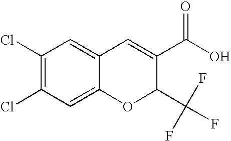 |
| | 6,7-dichloro-2-trifluoromethyl-2H-1-benzopyran-3- |
| | carboxylic acid; |
| B-46 | 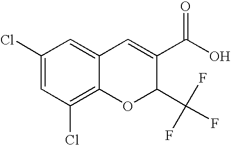 |
| | 6,8-dichloro-2-trifluoromethyl-2H-1-benzopyran-3- |
| | carboxylic acid; |
| B-47 | 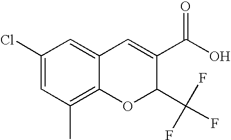 |
| | 6-chloro-8-methyl-2-trifluoromethyl-2H-1-benzopyran- |
| | 3-carboxylic acid; |
| B-48 | 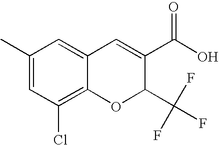 |
| | 8-chloro-6-methyl-2-trifluoromethyl-2H-1-benzopyran-3- |
| | carboxylic acid; |
| B-49 | 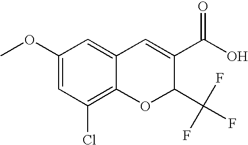 |
| | 8-chloro-6-methoxy-2-trifluoromethyl-2H-1-benzopyran- |
| | 3-carboxylic acid; |
| B-50 | 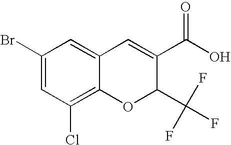 |
| | 6-bromo-8-chloro-2-trifluoromethyl-2H-1-benzopyran-3- |
| | carboxylic acid; |
| B-51 | 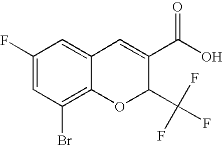 |
| | 8-bromo-6-fluoro-2-trifluoromethyl-2H-1-benzopyran-3- |
| | carboxylic acid; |
| B-52 | 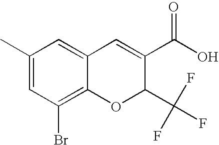 |
| | 8-bromo-6-methyl-2-trifluoromethyl-2H-I-benzopyran-3- |
| | carboxylic acid; |
| B-53 | 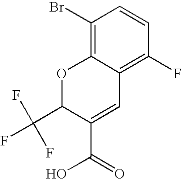 |
| | 8-bromo-5-fluoro-2-trifluoromethyl-2H-1-benzopyran-3- |
| | carboxylic acid; |
| B-54 | 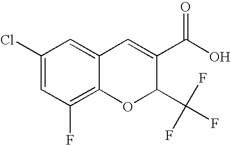 |
| | 6-chloro-8-fluoro-2-trifluoromethyl-2H-1-benzopyran- |
| | 3-carboxylic acid; |
| B-55 | 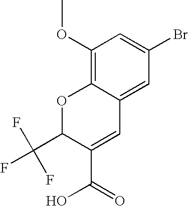 |
| | 6-bromo-8-methoxy-2-trifluoromethyl-2H-1-benzopyran- |
| | 3-carboxylic acid; |
| B-56 | 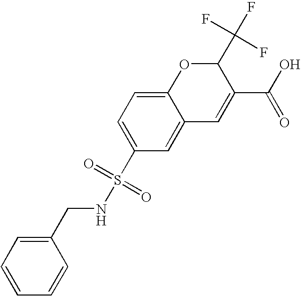 |
| | 6-[[(phenylmethyl)amino]sulfonyl]-2-trifluoromethyl- |
| | 2H-1-benzopyran-3-carboxylic acid; |
| B-57 | 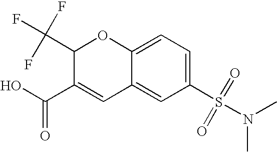 |
| | 6-[(dimethylamino)sulfonyl]-2-trifluoromethyl-2H- |
| | 1-benzopyran-3-carboxylic acid; |
| B-58 | 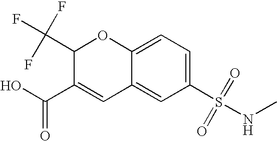 |
| | 6-[(methylamino)sulfonyl]-2-trifluoromethyl-2H- |
| | 1-benzopyran-3-carboxylic acid; |
| B-59 | 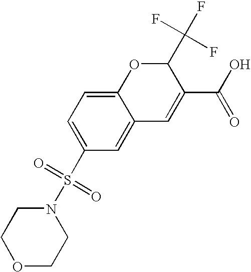 |
| | 6-[(4-morpholino)sulfonyl]-2-trifluoromethyl-2H- |
| | 1-benzopyran-3-carboxylic acid; |
| B-60 | 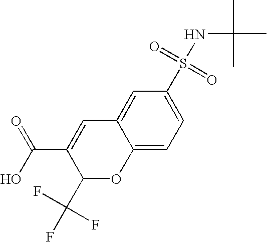 |
| | 6-[(1,1-dimethylethyl)aminosulfonyl]-2-trifluoromethyl- |
| | 2H-1-benzopyran-3-carboxylic acid; |
| B-61 | 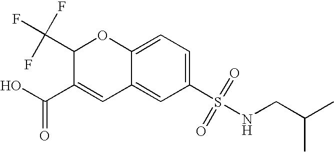 |
| | 6-[(2-methylpropyl)aminosulfonyl]-2-trifluoromethyl- |
| | 2H-1-benzopyran-3-carboxylic acid; |
| B-62 | 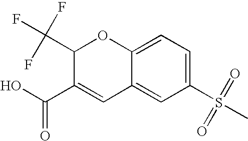 |
| | 6-methylsulfonyl-2-trifluoromethyl-2H-1-benzopyran-3- |
| | carboxylic acid; |
| B-63 | 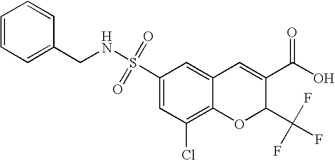 |
| | 8-chloro-6-[[(phenylmethyl)amino]sulfonyl]-2- |
| | trifluoromethyl-2H-1-benzopyran-3-carboxylic acid; |
| B-64 | 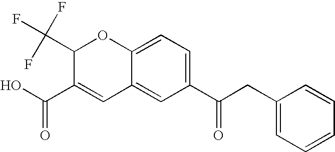 |
| | 6-phenylacetyl-2-trifluoromethyl-2H-1-benzopyran-3- |
| | carboxylic acid; |
| B-65 | 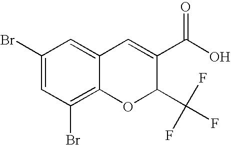 |
| | 6,8-dibromo-2-trifluoromethyl-2H-1-benzopyran-3- |
| | carboxylic acid; |
| B-66 | 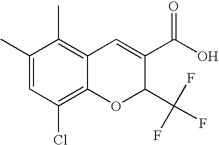 |
| | 8-chloro-5,6-dimethyl-2-trifluoromethyl-2H-1- |
| | benzopyran-3-carboxylic acid; |
| B-67 | 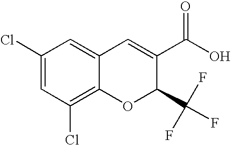 |
| | 6,8-dichloro-(S)-2-trifluoromethyl-2H-1-benzopyran- |
| | 3-carboxylic acid; |
| B-68 | 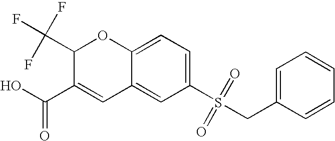 |
| | 6-benzylsulfonyl-2-trifluoromethyl-2H-1-benzopyran- |
| | 3-carboxylic acid; |
| B-69 | 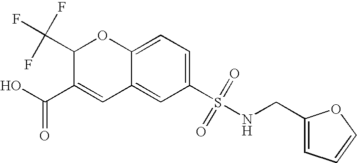 |
| | 6-[[N-(2-furylmethyl)amino]sulfonyl]-2- |
| | trifluoromethyl-2H-1-benzopyran-3-carboxylic acid; |
| B-70 | 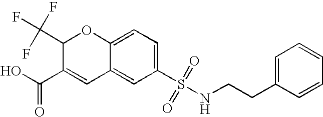 |
| | 6-[[N-(2-phenylethyl)amino]sulfonyl]-2- |
| | trifluoromethyl-2H-1-benzopyran-3-carboxylic acid; |
| B-71 | 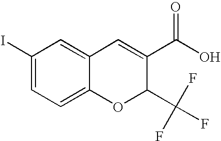 |
| | 6-iodo-2-trifluoromethyl-2H-1-benzopyran-3- |
| | carboxylic acid; |
| B-72 | 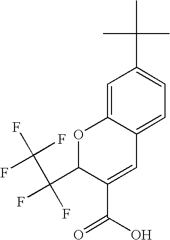 |
| | 7-(1,1-dimethylethyl)-2-pentafluoroethyl-2H- |
| | 1-benzopyran-3-carboxylic acid; |
| B-73 | 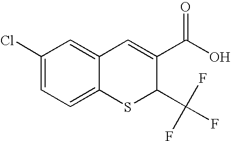 |
| | 6-chloro-2-trifluoromethyl-2H-1-benzothiopyran- |
| | 3-carboxylic acid; |
| B-74 | 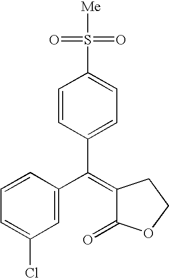 |
| | 3-[(3-chloro-phenyl)-(4-methanesulfonyl-phenyl)- |
| | methylene]-dihydro-furan-2-one; |
| B-75 | 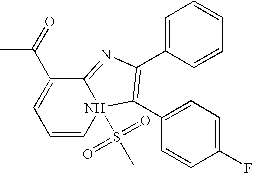 |
| | 8-acetyl-3-(4-fluoropbenyl)-2-(4-methyl- |
| | sulfonyl)phenyl-imidazo(1,2-a)pyridine; |
| B-76 | 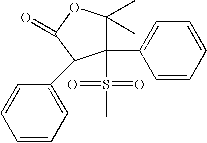 |
| | 5,5-dimethyl-4-(4-methylsulfonyl)phenyl-3-phenyl- |
| | 2-(5H)-furanone; |
| B-77 | 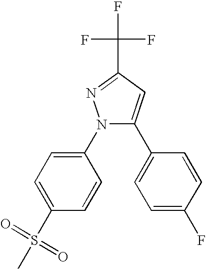 |
| | 5-(4-fluorophenyl)-1-[4-(methylsulfonyl)phenyl]-3- |
| | (trifluorometbyl)pyrazole; |
| B-78 | 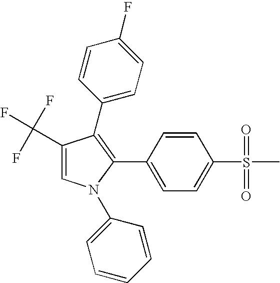 |
| | 4-(4-fluorophenyl)-5-[4-(methylsulfonyl)phenyl]- |
| | 1-phenyl-3-(trifluoromethyl)pyrazole; |
| B-79 | 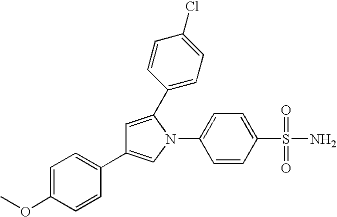 |
| | 4-(5-(4-chlorophenyl)-3-(4-methoxyphenyl)-1H- |
| | pyrazol-1-yl)benzenesulfonamide; |
| B-80 | 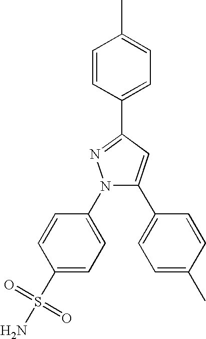 |
| | 4-(3,5-bis(4-methylphenyl)-1H-pyrazol-1- |
| | yl)benzenesulfonamide; |
| B-81 | 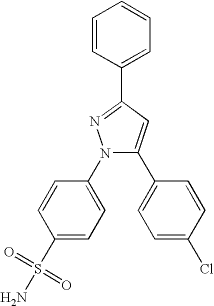 |
| | 4-(5-(4-chlorophenyl)-3-phenyl-1H-pyrazol-1- |
| | yl)benzenesulfonamide; |
| B-82 | 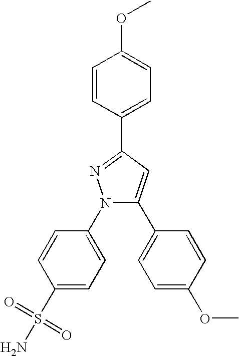 |
| | 4-(3,5-bis(4-methoxyphenyl)-1H-pyrazol-1- |
| | yl)benzenesulfonamide; |
| B-83 | 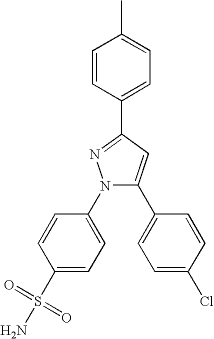 |
| | 4-(5-(4-chlorophenyl)-3-(4-methylphenyl)-1H- |
| | pyrazol-1-yl)benzenesulfonamide; |
| B-84 | 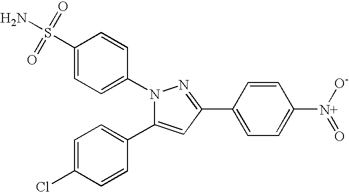 |
| | 4-(5-(4-chlorophenyl)-3-(4-nitropbenyl)-1H-pyrazol- |
| | 1-yl)benzenesulfonamide; |
| B-85 | 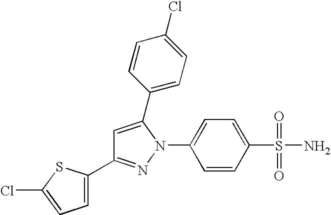 |
| | 4-(5-(4-chlorophenyl)-3-(5-chloro-2-thienyl)-1H- |
| | pyrazol-1-yl)benzenesulfonamide; |
| B-86 | 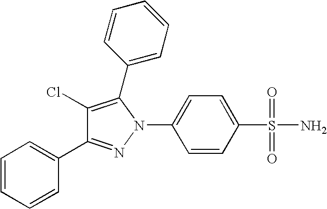 |
| | 4-(4-chloro-3,5-diphenyl-1H-pyrazol-1- |
| | yl)benzenesulfonamide; |
| B-87 | 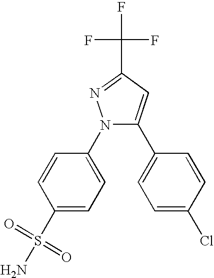 |
| | 4-[5-(4-chlorophenyl)-3-(trifluoromethyl)-1H-pyrazol- |
| | 1-yl]benzenesulfonamide; |
| B-88 | 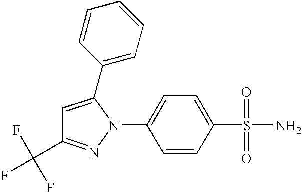 |
| | 4-[5-phenyl-3-(trifluorometbyl)-1H-pyrazol-1- |
| | yl]benzenesulfonamide; |
| B-89 | 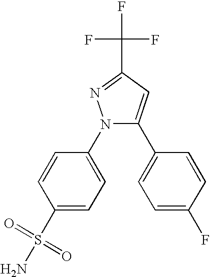 |
| | 4-[5-(4-fluorophenyl)-3-(trifluoromethyl)-1H-pyrazol- |
| | 1-yl]benzenesulfonamide; |
| B-90 | 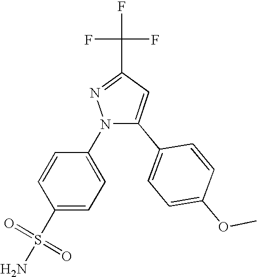 |
| | 4[5-(4-methoxyphenyl)-3-(trifluoromethyl)-1H-pyrazol- |
| | 1-yl]benzenesulfonamide; |
| B-91 | 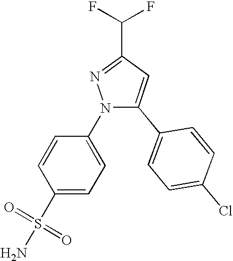 |
| | 4-[5-(4-chlorophenyl)-3-(difluoromethyl)-1H- |
| | pyrazol-1-yl]benzenesulfonamide; |
| B-92 | 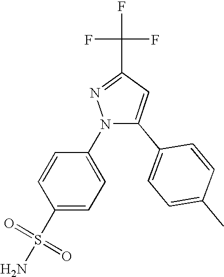 |
| | 4-[5-(4-methylphenyl)-3-(trifluoromethyl)-1H-pyrazol- |
| | 1-yl]benzenesulfonamide; |
| B-93 | 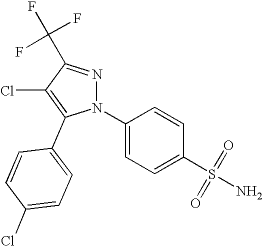 |
| | 4-[4-chloro-5-(4-chlorophenyl)-3-(trifluoromethyl)- |
| | 1H-pyrazol-1-yl]benzenesulfonamide; |
| B-94 | 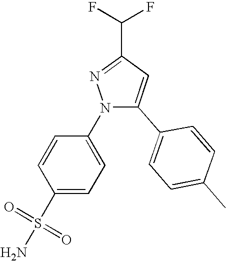 |
| | 4-[3-(difluoromethyl)-5-(4-methylphenyl)-1H- |
| | pyrazol-1-yl]benzenesulfonamide; |
| B-95 | 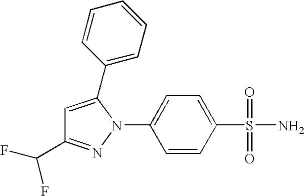 |
| | 4-[3-(difluoromethyl)-5-phenyl-1H-pyrazol-1- |
| | yl]benzenesulfonamide; |
| B-96 | 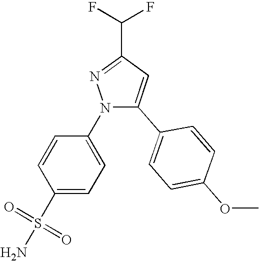 |
| | 4-[3-(difluoromethyl)-5-(4-methoxyphenyl)-1H- |
| | pyrazol-1-yl]benzenesulfonamide; |
| B-97 | 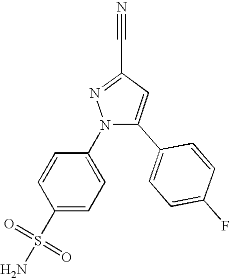 |
| | 4-[3-cyano-5-(4-fluorophenyl)-1H-pyrazol-1- |
| | yl]benzenesulfonamide; |
| B-98 | 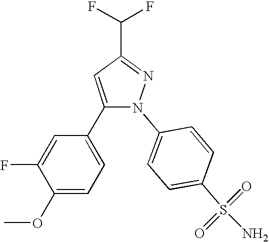 |
| | 4-[3-(difluoromethyl)-5-(3-fluoro-4-methoxyphenyl)- |
| | 1H-pyrazol-1-yl]benzenesulfonamide; |
| B-99 | 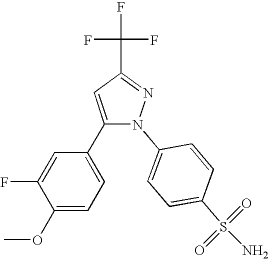 |
| | 4-[5-(3-fluoro-4-methoxyphenyl)-3-(trifluoromethyl)- |
| | 1H-pyrazol-1-yl]benzenesulfonamide; |
| B-100 | 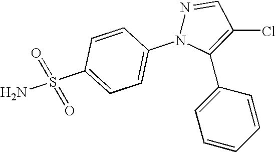 |
| | 4-[4-chloro-5-phenyl-1H-pyrazol-1- |
| | yl]benzenesulfonamide; |
| B-101 | 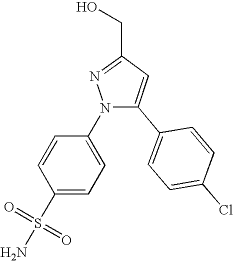 |
| | 4-[5-(4-chlorophenyl)-3-(hydroxymetbyl)-1H-pyrazol-1- |
| | yl]benzenesulfonamide; |
| B-102 | 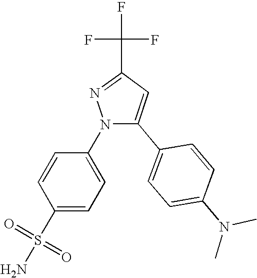 |
| | 4-[5-(4-(N,N-dimethylamino)phenyl)-3-(trifluoromethyl)- |
| | 1H-pyrazol-1-yl]benzenesulfonamide; |
| B-103 | 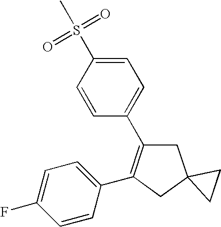 |
| | 5-(4-fluorophenyl)-6-[4-(methylsulfonyl)phenyl] |
| | spiro[2.4]hept-5-ene; |
| B-104 | 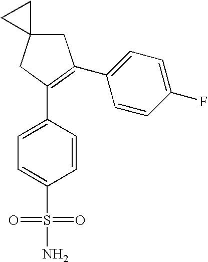 |
| | 4-[6-(4-fluorophenyl)spiro[2.4]hept-5-en-5- |
| | yl]benzenesulfonamide; |
| B-105 | 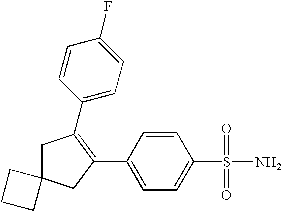 |
| | 6-(4-fluorophenyl)-7-[4-methylsulfonyl)phenyl] |
| | spiro[3.4]oct-6-ene; |
| B-106 | 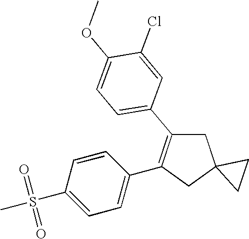 |
| | 5-(3-chloro-4-methoxyphenyl)-6-[4- |
| | (methylsulfonyl)phenyl]spiro[2.4]hept-5-ene; |
| B-107 | 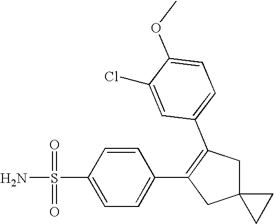 |
| | 4-[6-(3-chloro-4-methoxyphenyl)spiro[2.4]hept- |
| | 5-en-5-yl]benzenesulfonamide; |
| B-108 | 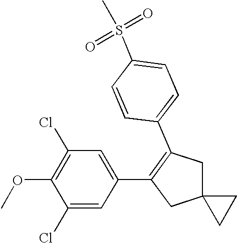 |
| | 5-(3,5-dichloro-4-methoxyphenyl)-6-[4- |
| | (methylsulfonyl)phenyl]spiro[2.4]hept-5-ene; |
| B-109 | 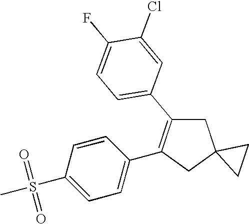 |
| | 5-(3-chloro-4-fluorophenyl)-6-[4- |
| | (methylsulfonyl)phenyl]spiro[2.4]hept-5-ene; |
| B-110 | 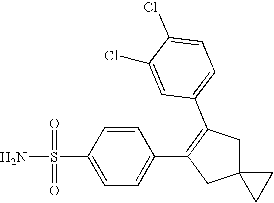 |
| | 4-[6-(3,4-dichlorophenyl)spiro[2.4]hept-5-en- |
| | 5-yl]benzenesulfonamide; |
| B-111 | 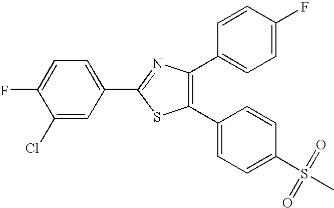 |
| | 2-(3-chloro-4-fluorophenyl)-4-(4-fluorophenyl)- |
| | 5-(4-methylsulfonylphenyl)thiazole; |
| B-112 | 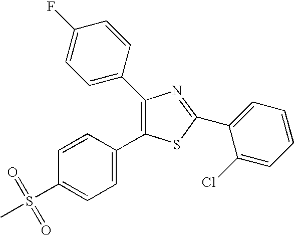 |
| | 2-(2-chlorophenyl)-4-(4-fluorophenyl)-5- |
| | (4-methylsulfonylphenyl)thiazole; |
| B-113 | 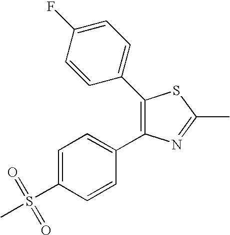 |
| | 5-(4-fluorophenyl)-4-(4-methylsulfonylphenyl)-2- |
| | methylthiazole; |
| B-114 | 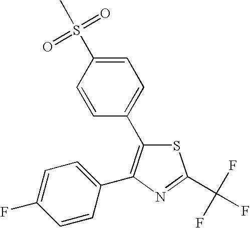 |
| | 4-(4-fluorophenyl)-5-(4-methylsulfonylphenyl)-2- |
| | trifluoromethylthiazole; |
| B-115 | 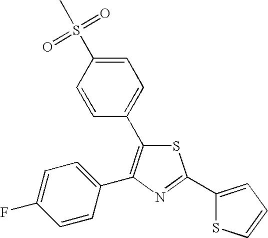 |
| | 4-(4-fluorophenyl)-5-(4-methylsulfonylphenyl)- |
| | 2-(2-thienyl)thiazole; |
| B-116 | 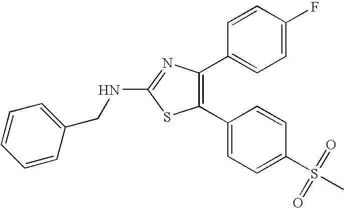 |
| | 4-(4-fluorophenyl)-5-(4-methylsulfonylphenyl)-2- |
| | benzylaminothiazole; |
| B-117 | 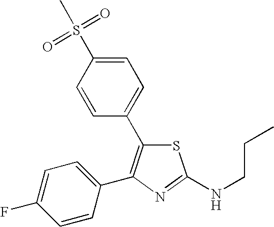 |
| | 4-(4-fluorophenyl)-5-(4-methylsulfonylphenyl)-2- |
| | (1-propylamino)thiazole; |
| B-118 | 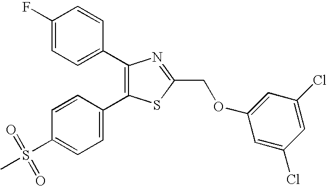 |
| | 2-((3,5-dichlorophenoxy)methyl)-4-(4-fluorophenyl)- |
| | 5-[4-(methylsulfonyl)phenyl]thiazole; |
| B-119 | 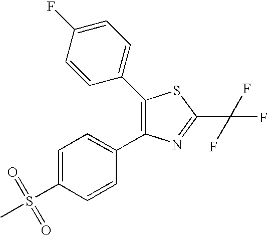 |
| | 5-(4-fluorophenyl)-4-(4-methylsulfonylphenyl)- |
| | 2-trifluoromethylthiazole; |
| B-120 | 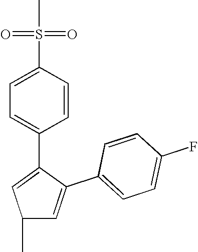 |
| | 1-methylsulfonyl-4-[1,1-dimetbyl-4-(4-fluorophenyl) |
| | cyclopenta-2,4-dien-3-yl]benzene; |
| B-121 | 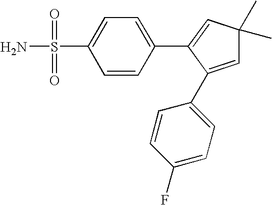 |
| | 4-[4-(4-fluorophenyl)-1,1-dimethylcyclopenta-2,4- |
| | dien-3-yl]benzenesulfonamide; |
| B-122 | 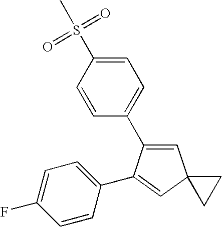 |
| | 5-(4-fluorophenyl)-6-[4-(methylsulfonyl) |
| | phenyl]spiro[2.4]hepta-4,6-diene; |
| B-123 | 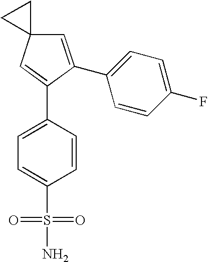 |
| | 4-[6-(4-fluorophenyl)spiro[2.4]hepta-4,6- |
| | dien-5-yl]benzenesulfonamide; |
| B-124 | 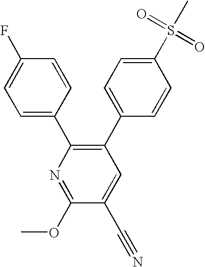 |
| | 6-(4-fluorophenyl)-2-methoxy-5-[4-(methylsulfonyl) |
| | phenyl]-pyridine-3-carbonitrile; |
| B-125 | 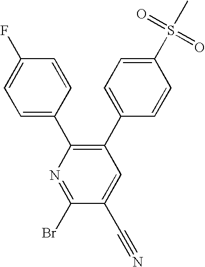 |
| | 2-bromo-6-(4-fluorophenyl)-5-[4- |
| | (methylsulfonyl)phenyl]-pyridine-3-carbonitrile; |
| B-126 | 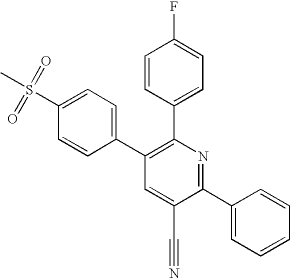 |
| | 6-(4-fluorophenyl)-5-[4-(methylsulfonyl)phenyl]-2- |
| | phenyl-pyridine-3-carbonitrile; |
| B-127 | 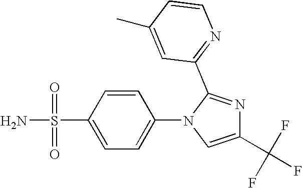 |
| | 4-[2-(4-methylpyridin-2-yl)-4-(trifluoromethyl)-1H- |
| | imidazol-1-yl]benzenesulfonamide; |
| B-128 | 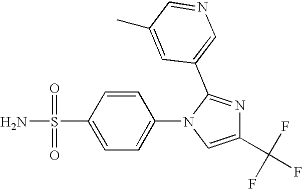 |
| | 4-[2-(5-methylpyridin-3-yl)-4-(trifluoromethyl)-1H- |
| | imidazol-1-yl]benzenesulfonamide; |
| B-129 | 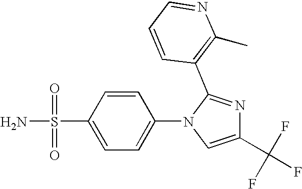 |
| | 4-[2-(2-methylpyridin-3-yl)-4-(trifluoromethyl)-1H- |
| | imidazol-1-yl]benzenesulfonamide; |
| B-130 | 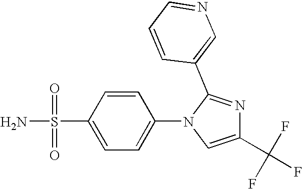 |
| | 3-[1-[4-(methylsulfonyl)phenyl]-4-(trifluoromethyl)- |
| | 1H-imidazol-2-yl]pyridine; |
| B-131 | 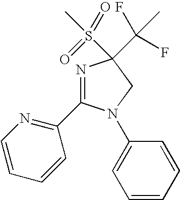 |
| | 2-[1-[4-(methylsulfonyl)phenyl-4- |
| | (trifluoromethyl)]-1H-imidazol-2-yl]pyridine; |
| B-132 | 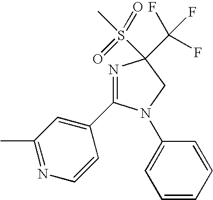 |
| | 2-methyl-4-[1-[4-(methylsulfonyl)phenyl-4- |
| | (trifluoromethyl)]-1H-imidazol-2-yl]pyridine; |
| B-133 | 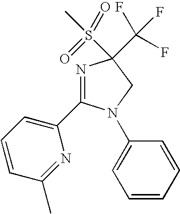 |
| | 2-methyl-6-[1-[4-(methylsulfonyl)phenyl-4- |
| | (trifluoromethyl)]-1H-imidazol-2-yl]pyridine; |
| B-134 | 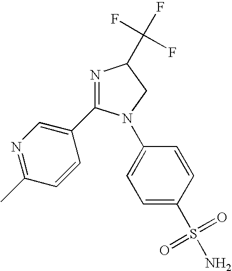 |
| | 4-[2-(6-methylpyridin-3-yl)-4-(trifluoromethyl)- |
| | 1H-imidazol-1-yl]benzenesulfonamide; |
| B-135 | 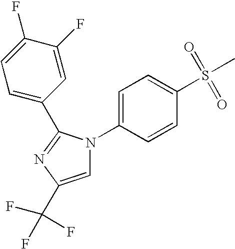 |
| | 2-(3,4-difluorophenyl)-1-[4-(methylsulfonyl) |
| | phenyl]-4-(trifluoromethyl)-1H-imidazole; |
| B-136 | 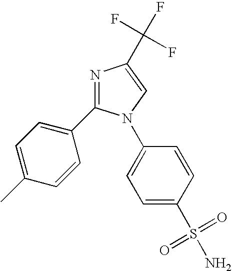 |
| | 4-[2-(4-methylphenyl)-4-(trifluoromethyl)-1H- |
| | imidazol-1-yl]benzenesulfonamide; |
| B-137 | 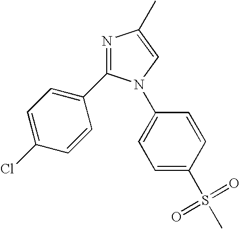 |
| | 2-(4-chlorophenyl)-1-[4-(methylsulfonyl)phenyl]-4- |
| | methyl-1H-imidazole; |
| B-138 | 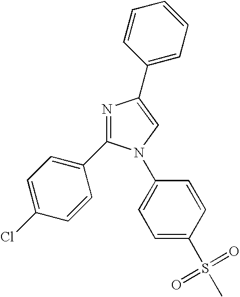 |
| | 2-(4-chlorophenyl)-1-[4-(methylsulfonyl)phenyl]-4- |
| | phenyl-1H-imidazole; |
| B-139 | 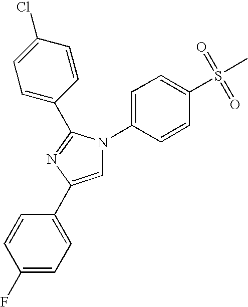 |
| | 2-(4-chlorophenyl)-4-(4-fluorophenyl)-1-[4- |
| | (methylsulfonyl)phenyl]-1H-imidazole; |
| B-140 | 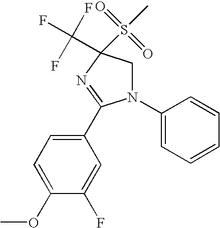 |
| | 2-(3-fluoro-4-methoxyphenyl)-1-[4-(methylsulfonyl) |
| | phenyl-4-(trifluoromethyl)]-1H-imidazole; |
| B-141 | 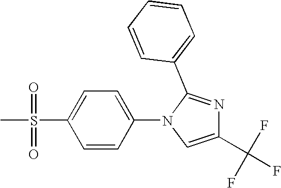 |
| | 1-[4-(methylsulfonyl)phenyl]-2-phenyl-4- |
| | trifluoromethyl-1H-imidazole; |
| B-142 | 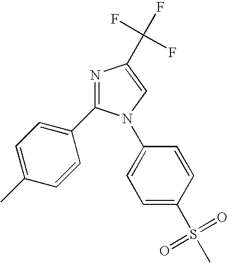 |
| | 2-(4-methylphenyl)-1-[4-(methylsulfonyl)phenyl]- |
| | 4-trifluoromethyl-1H-imidazole; |
| B-143 | 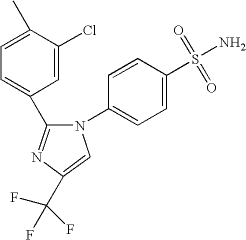 |
| | 4-[2-(3-chloro-4-methylphenyl)-4-(trifluoromethyl)- |
| | 1H-imidazol-1-yl]benzenesulfonamide; |
| B-144 | 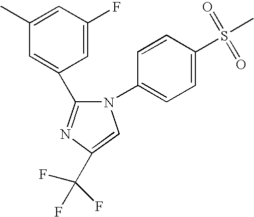 |
| | 2-(3-fluoro-5-methylphenyl)-1-[4-(methylsulfonyl) |
| | phenyl]-4-(trifluoromethyl)-1H-imidazole; |
| B-145 | 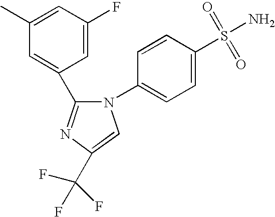 |
| | 4-[2-(3-fluoro-5-methylphenyl)-4-(trifluoromethyl- |
| | 1H-imidazole-1-yl]benzenesulfonamide; |
| B-146 | 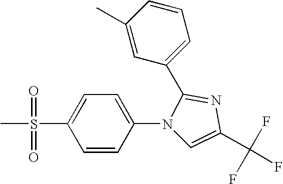 |
| | 2-(3-methylphenyl)-1-[4-(methylsulfonyl)phenyl]- |
| | 4-trifluoromethyl-1H-imidazole; |
| B-147 | 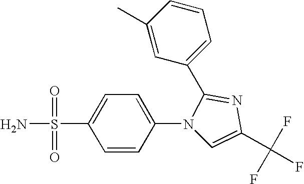 |
| | 4-[2-(3-methylphenyl)-4-trifluoromethyl-1H-imidazol- |
| | 1-yl]benzenesulfonamide; |
| B-148 | 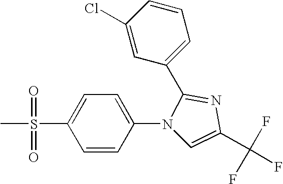 |
| | 1-[4-(methylsulfonyl)phenyl]-2-(3-chlorophenyl)-4- |
| | trifluoromethyl-1H-imidazole |
| B-149 | 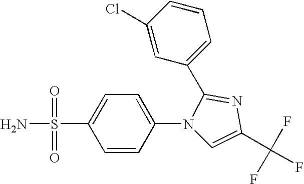 |
| | 4-[2-(3-chlorophenyl)-4-trifluoromethyl-1H-imidazol-1- |
| | yl]benzenesulfonamide; |
| B-150 | 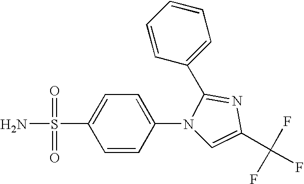 |
| | 4-[2-phenyl-4-trifluoromethyl-1H-imidazol-1- |
| | yl]benzenesulfonamide; |
| B-151 | 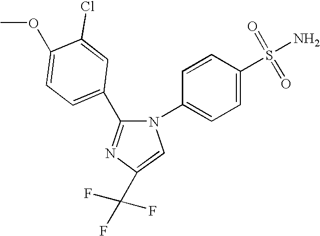 |
| | 4-[2-(4-methoxy-3-chlorophenyl)-4-trifluoromethyl-1H- |
| | imidazol-1-yl]benzenesulfonamide; |
| B-152 | 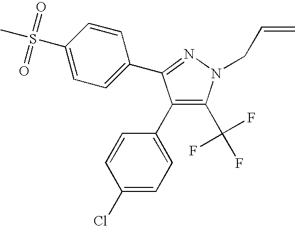 |
| | 1-allyl-4-(4-fluorophenyl)-3-[4-(methylsulfonyl)phenyl]- |
| | 5-(trifluoromethyl)-1H-pyrazole; |
| B-153 | 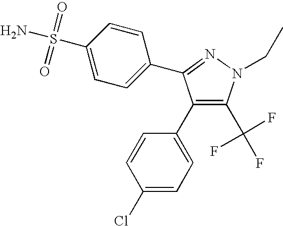 |
| | 4-[1-ethyl-4-(4-fluorophenyl)-5-(trifluoromethyl)-1H- |
| | pyrazol-3-yl]benzenesulfonamide; |
| B-154 | 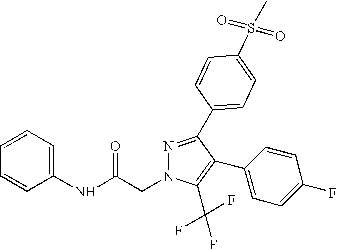 |
| | N-phenyl-[4-(4-fluorophenyl)-3-[4- |
| | (methylsulfonyl)phenyl]-5-(trifluoromethyl)-1H- |
| | pyrazol-1-yl]acetamide; |
| B-155 | 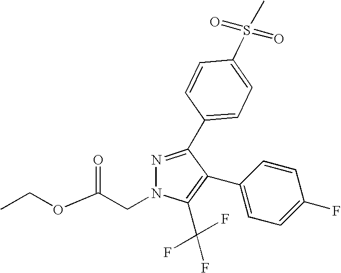 |
| | ethyl[4-(4-fluorophenyl)-3-[4-(methylsulfonyl) |
| | phenyl]-5-(trifluoromethyl)-1H-pyrazol-1-yl]acetate; |
| B-156 | 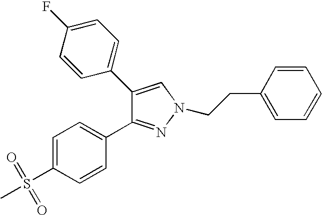 |
| | 4-(4-fluorophenyl)-3-[4-(methylsulfonyl)phenyl]- |
| | 1-(2-phenylethyl)-1H-pyrazole; |
| B-157 | 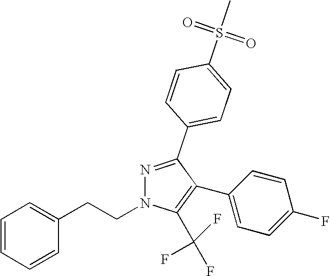 |
| | 4-(4-fluorophenyl)-3-[4-(methylsulfonyl)phenyl]- |
| | 1-(2-phenylethyl)-5-(trifluoromethyl)pyrazole; |
| B-158 | 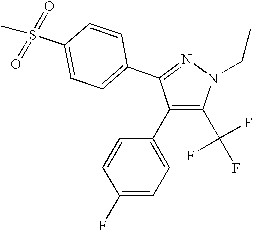 |
| | 1-ethyl-4-(4-fluorophenyl)-3-[4-methylsulfonyl)phenyl]- |
| | 5-(trifluoromethyl)-1H-pyrazole; |
| B-159 | 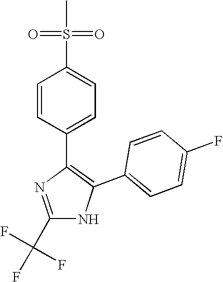 |
| | 5-(4-fluorophenyl)-4-(4-methylsulfonylphenyl)- |
| | 2-trifluoromethyl-1H-imidazole; |
| B-160 | 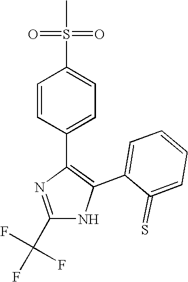 |
| | 4-[4-(methylsulfonyl)phenyl]-5-(2-thiophenyl)- |
| | 2-(trifluoromethyl)-1H-imidazole; |
| B-161 | 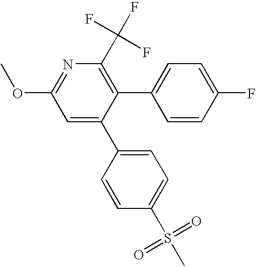 |
| | 5-(4-fluoropheriyl)-2-methoxy-4-[4-(methylsulfonyl) |
| | phenyl]-6-(trifluoromethyl)pyridine; |
| B-162 | 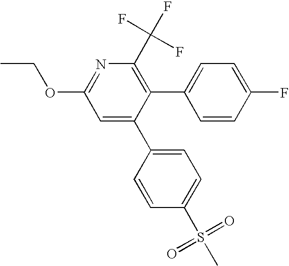 |
| | 2-ethoxy-5-(4-fluorophenyl)-4-[4-(methylsulfonyl) |
| | phenyl]-6-(trifluoromethyl)pyridine; |
| B-163 | 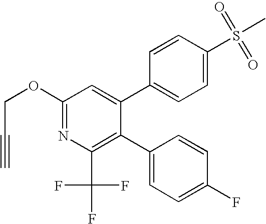 |
| | 5-(4-fluorophenyl)-4-[4-(methylsulfonyl)phenyl]- |
| | 2-(2-propynyloxy)-6-(trifluoromethyl)pyridine; |
| B-164 | 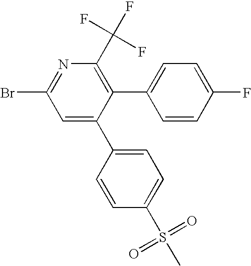 |
| | 2-bromo-5-(4-fluorophenyl)-4-[4-(methylsulfonyl)phenyl]- |
| | 6-(trifluoromethyl)pyridine; |
| B-165 | 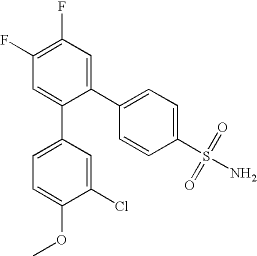 |
| | 4-[2-(3-chloro-4-methoxyphenyl)-4,5- |
| | difluorophenyl]benzenesulfonamide; |
| B-166 | 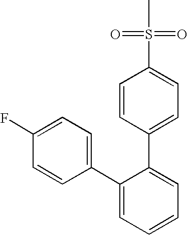 |
| | 1-(4-fluorophenyl)-2-[4-methylsulfonyl)phenyl] |
| | benzene; |
| B-167 | 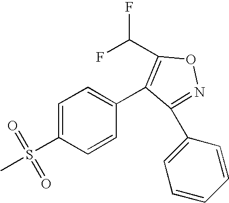 |
| | 5-difluoromethyl-4-(4-methylsulfonylphenyl)-3- |
| | phenylisoxazole; |
| B-168 | 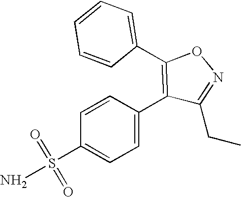 |
| | 4-[3-ethyl-5-phenylisoxazol-4-yl]benzenesulfonamide; |
| B-169 | 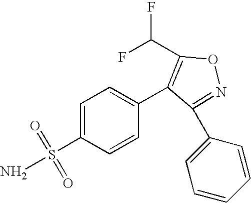 |
| | 4-[5-difluoromethyl-3-phenylisoxazol-4- |
| | yl]benzenesulfonamide; |
| B-170 | 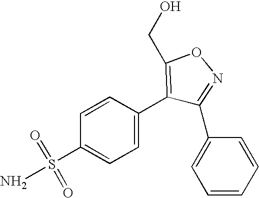 |
| | 4-[5-hydroxymethyl-3-phenylisoxazol-4- |
| | yl]benzenesulfonamide; |
| B-171 | 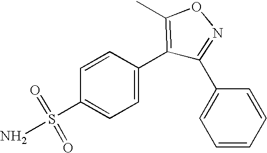 |
| | 4-[5-methyl-3-phenyl-isoxazol-4- |
| | yl]benzenesulfonamide; |
| B-172 | 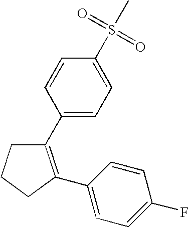 |
| | 1-[2-(4-fluorophenyl)cyclopenten-1-yl]-4- |
| | (methylsulfonyl)benzene; |
| B-173 | 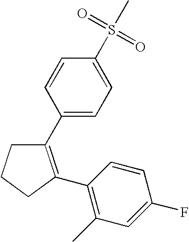 |
| | 1-[2-(4-fluoro-2-methylphenyl)cyclopenten-1- |
| | yl]-4-(methylsulfonyl)benzene; |
| B-174 | 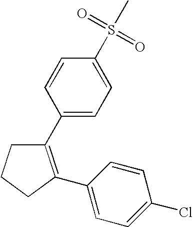 |
| | 1-[2-(4-chlorophenyl)cyclopenten-1-yl]-4- |
| | (methylsulfonyl)benzene; |
| B-175 | 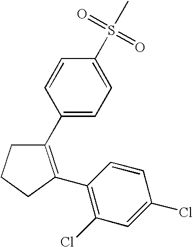 |
| | 1-[2-(2,4-dichlorophenyl)cyclopenten-1-yl]- |
| | 4-(methylsulfonyl)benzene; |
| B-176 | 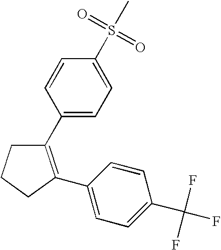 |
| | 1-[2-(4-trifloromethylphenyl)cyclopenten-1- |
| | yl]-4-(methylsulfonyl)benzene; |
| B-177 | 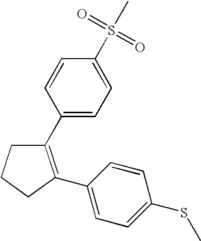 |
| | 1-[2-(4-methylthiophenyl)cyclopenten-1- |
| | yl]-4-(methylsulfonyl)benzene; |
| B-178 | 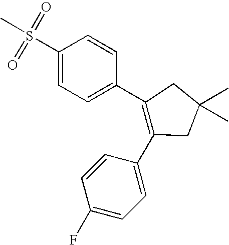 |
| | 1-[2-(4-fluorophenyl)-4,4-dimethylcyclopenten- |
| | 1-yl]-4-(methylsulfonyl)benzene; |
| B-179 | 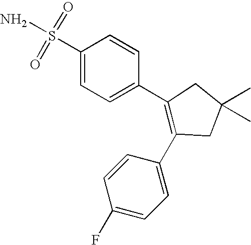 |
| | 4-[2-(4-fluorophenyl)-4,4-dimethylcyclopenten- |
| | 1-yl]benzenesulfonamide; |
| B-180 | 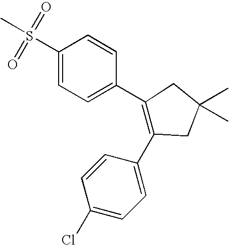 |
| | 1-[2-(3-chlorophenyl)-4,4-dimethylcyclopenten-1- |
| | yl]-4-(methylsulfonyl)benzene; |
| B-181 | 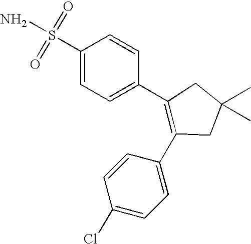 |
| | 4-[2-(4-chlorophenyl)-4,4-dimethylcyclopenten-1- |
| | yl]benzenesulfonamide; |
| B-182 | 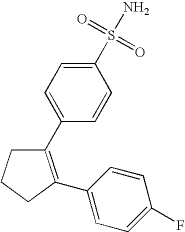 |
| | 4-[2-(4-fluorophenyl)cyclopenten-1- |
| | yl]benzenesulfonamide; |
| B-183 | 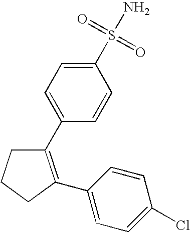 |
| | 4-[2-(4-chlorophenyl)cyclopenten-1- |
| | yl]benzenesulfonamide; |
| B-184 | 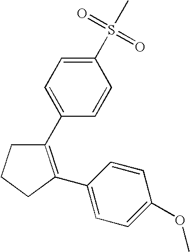 |
| | 1-[2-(4-methoxyphenyl)cyclopenten-1- |
| | yl]-4-(methylsulfonyl)benzene; |
| B-185 | 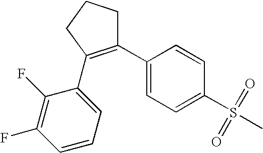 |
| | 1-[2-(2,3-difluorophenyl)cyclopenten-1- |
| | yl]-4-(methylsulfonyl)benzene; |
| B-186 | 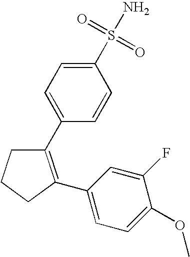 |
| | 4-[2-(3-fluoro-4-methoxyphenyl)cyclopenten-1- |
| | yl]benzenesulfonamide; |
| B-187 | 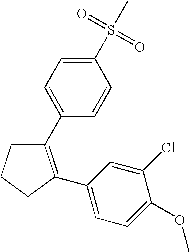 |
| | 1-[2-(3-chloro-4-methoxyphenyl)cyclopenten-1- |
| | yl]-4-(methylsulfonyl)benzene; |
| B-188 | 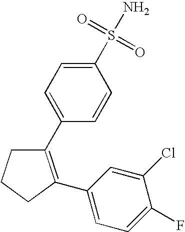 |
| | 4-[2-(3-chloro-4-fluorophenyl)cyclopenten-1- |
| | yl]benzenesulfonamide; |
| B-189 | 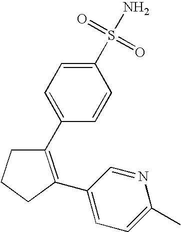 |
| | 4-[2-(2-methylpyridin-5-yl)cyclopenten-1- |
| | yl]benzenesulfonamide; |
| B-190 | 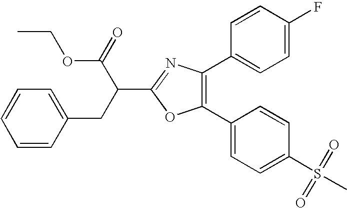 |
| | ethyl 2-[4-(4-fluoropbenyl)-5-[4- |
| | (methylsulfonyl)phenyl]oxazal-2-yl]-2- |
| | benzyl-acetate; |
| B-191 | 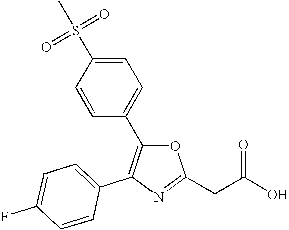 |
| | 2-[4-(4-fluorophenyl)-5-[4- |
| | (methylsulfonyl)phenyl]oxazol-2- |
| | yl]acetic acid; |
| B-192 | 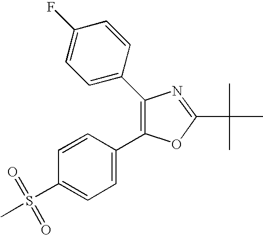 |
| | 2-(tert-butyl)-4-(4-fluorophenyl)-5-[4- |
| | (methylsulfonyl)phenyl]oxazole; |
| B-193 | 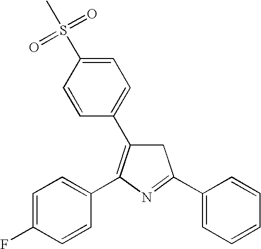 |
| | 4-(4-fluorophenyl)-5-[4-(methylsulfonyl) |
| | phenyl]-2-phenyloxazole, |
| B-194 | 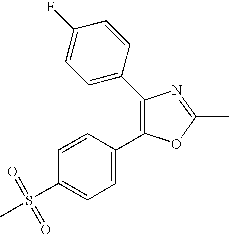 |
| | 4-(4-fluorophenyl)-2-methyl-5-[4- |
| | (methylsulfonyl)phenyl]oxazole; |
| B-195 | 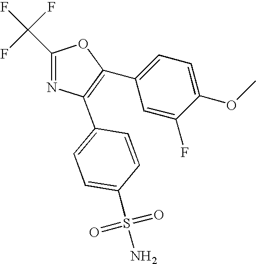 |
| | 4-[5-(3-fluoro-4-methoxyphenyl)-2- |
| | trifluoromethyl-4-oxazolyl]benzenesulfonamide; |
| B-196 | 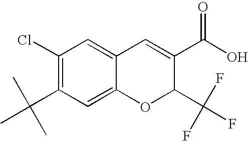 |
| | 6-chloro-7-(1,1-dimethylethyl)-2-trifluoromethyl- |
| | 2H-1-benzopyran-3-carboxylic acid; |
| B-197 | 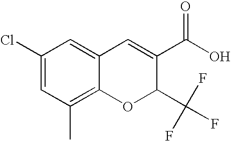 |
| | 6-chloro-8-methyl-2-trifluoromethyl-2H-1- |
| | benzopyran-3-carboxylic acid; |
| B-198 | 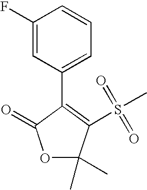 |
| | 5,5-dimethyl-3-(3-fluorophenyl)-4-methylsulfonyl- |
| | 2(5H)-furanone; |
| B-199 | 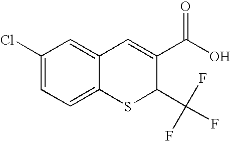 |
| | 6-chloro-2-trifluoromethyl-2H-1-benzothiopyran-3- |
| | carboxylic acid; |
| B-200 | 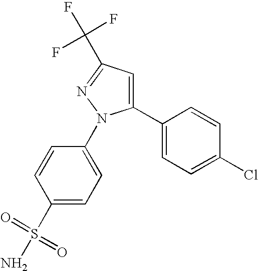 |
| | 4-[5-(4-chlorophenyl)-3-(trifluoromethyl)-1H- |
| | pyrazol-1-yl]benzenesulfonamide; |
| B-201 | 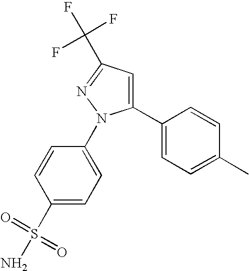 |
| | 4-[5-(4-methylphenyl)-3-(trifluoromethyl)-1H- |
| | pyrazol-1-yl]benzenesulfonamide; |
| B-202 | 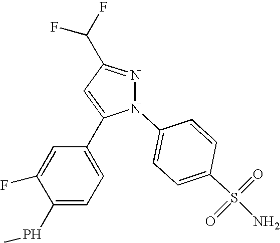 |
| | 4-[5-(3-fluoro-4-methoxyphenyl)-3-(difluoromethyl)- |
| | 1H-pyrazol-1-yl]benzenesulfonamide; |
| B-203 | 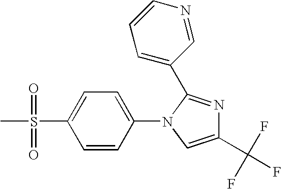 |
| | 3-[1-[4-(methylsulfonyl)phenyl]-4- |
| | trifluoromethyl-1H-imidazol-2-yl)pyridine; |
| B-204 | 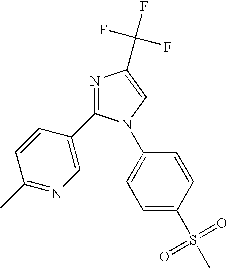 |
| | 2-methyl-5-[1-[4-(methylsulfonyl)phenyl]-4- |
| | trifluoromethyl-1H-imidazol-2-yl]pyridine; |
| B-205 | 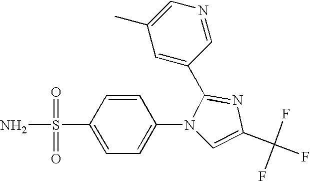 |
| | 4-[2-(5-methylpyridin-3-yl)-4-(trifluoromethyl)- |
| | 1H-imidazol-1-yl]benzenesulfonamide; |
| B-206 | 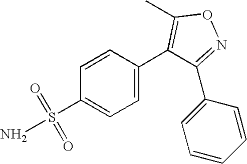 |
| | 4-[5-methyl-3-phenylisoxazol-4- |
| | yl]benzenesulfonamide; |
| B-207 | 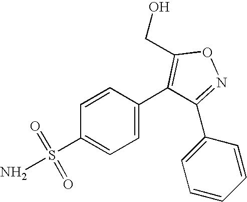 |
| | 4-[5-hydroxymethyl-3-phenylisoxazol-4- |
| | yl]benzenesulfonamide; |
| B-208 | 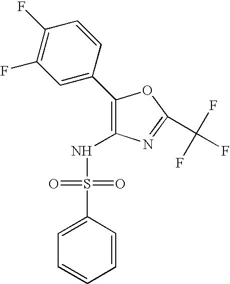 |
| | [2-trifluoromethyl-5-(3,4-difluorophenyl)-4- |
| | oxazolyl]benzenesulfonamide; |
| B-209 | 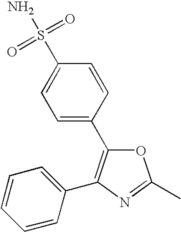 |
| | 4-[2-methyl-4-phenyl-5- |
| | oxazolyl]benzenesulfonamide; |
| B-210 | 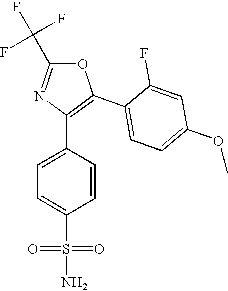 |
| | 4-[5-(2-fluoro-4-methoxyphenyl)-2- |
| | trifluoromethy]-4-oxazolyl]benzenesulfonamide; |
| B-211 | 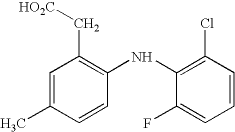 |
| B-212 | 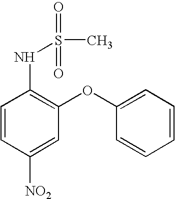 |
| | N-(4-nitro-2-phenoxy-phenyl)- |
| | methanesulfonamide or Nimesulide |
| B-213 | 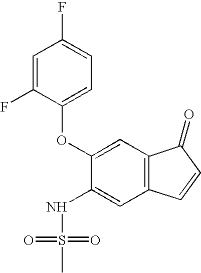 |
| | N-[6-(2,4-difluoro-phenoxy)-1-oxo-inden-5-yl]- |
| | methanesulfonamide or Flosulide |
| B-214 | 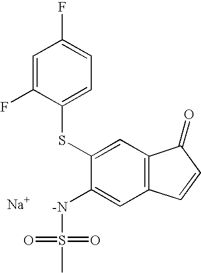 |
| | N-[6-(2,4-difluoro-phenylsulfanyl)-1-oxo-1H-inden-5- |
| | yl]-methanesulfonamide, soldium salt, or L-745337 |
| B-215 | 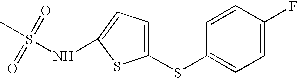 |
| | N-[5-(4-fluoro-phenylsulfanyl)-thiophen-2- |
| | yl]-methanesulfonamide |
| B-216 | 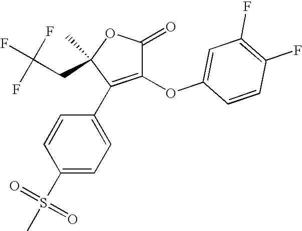 |
| | 3-(3,4-difluoro-phenoxy)-4-(4-methanesulfonyl- |
| | phenyl)-5-methyl-5-(2,2,2-trifluoro-ethyl)-5H- |
| | furan-2-one |
| B-217 | 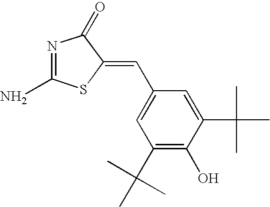 |
| | (5Z)-2-amino-5-[[3,5-bis(1,1-dimethylethyl)-4- |
| | hydroxyphenyl]methylene]-4(5H)-thiazolone or |
| | Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 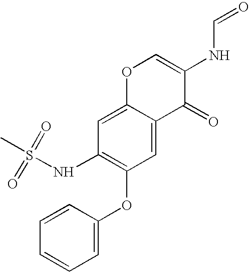 |
| | N-[3-(formylamino)-4-oxo-6-phenoxy-4H-1- |
| | benzopyran-7-yl]-methanesulfonamide or T614 |
| B-225 | D-1367 |
| B-226 | L-748731 |
| B-227 | 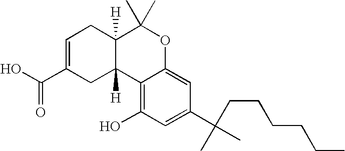 |
| | (6aR,10aR)-3-(1,1-dimethylheptyl)-6a,7,10,1Oa- |
| | tetrahydro-1-hydroxy-6,6-dimethyl-6H-dibenzo[b,d]pyran- |
| | 9-carboxylic acid or CT3 |
| B-228 | CGP-28238 |
| B-229 | 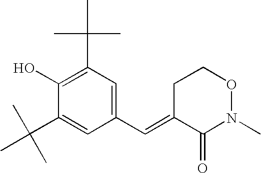 |
| | 4-[[3,5-bis(1,1-dimethylethyl)-4- |
| | hydroxyphenyl]methylene]dihydro-2-methyl- |
| | 2H-1 ,2-oxazin-3(4H)-one or BF-389 |
| B-230 | GR-253035 |
| B-231 | 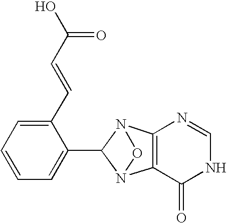 |
| | 2-(6-dioxo-9H-purin-8-yl)cinnamic acid |
| B-232 | S-2474 |
| B-233 | 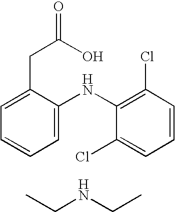 |
| B-234 | 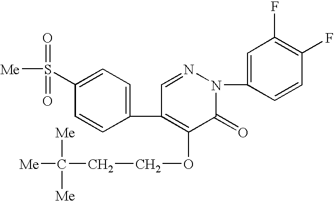 |
| B-235 | 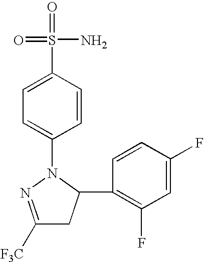 |
| B-236 | 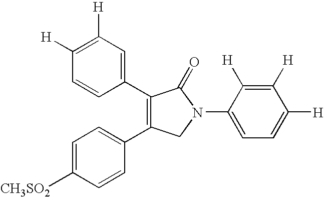 |
| B-237 | 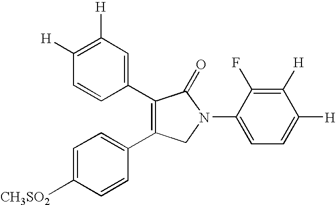 |
| B-238 | 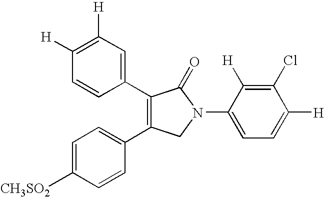 |
| B-239 | 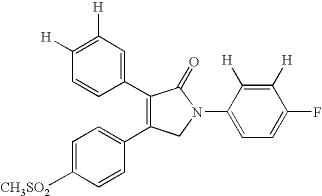 |
| B-240 | 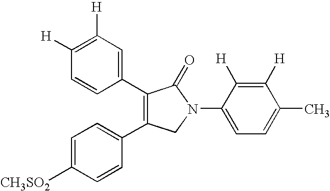 |
| B-241 | 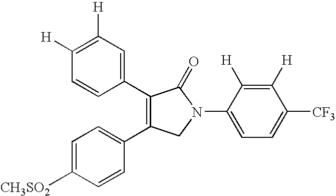 |
| B-242 | 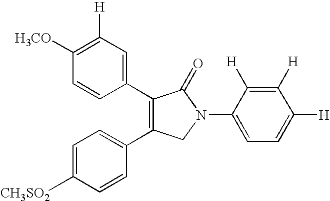 |
| B-243 | 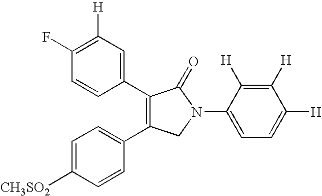 |
| B-244 | 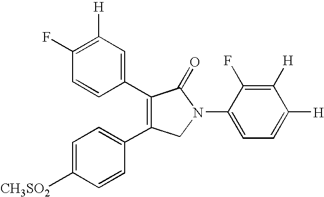 |
| B-245 | 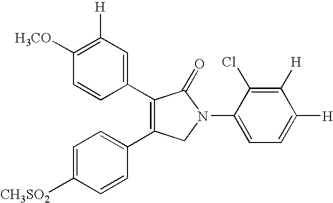 |
| B-246 | 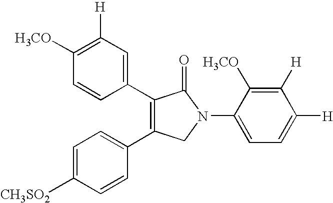 |
| B-247 | 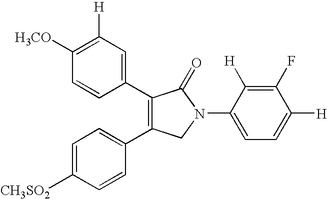 |
| B-248 | 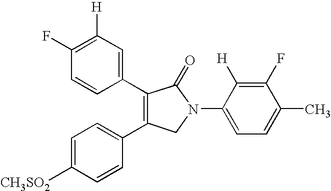 |
| B-249 | 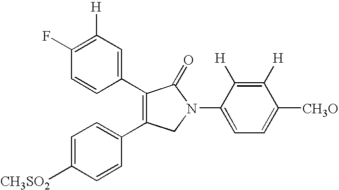 |
| B-250 | 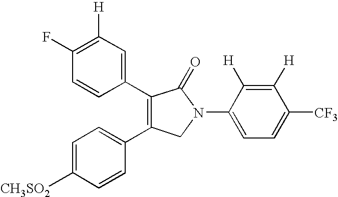 |
| B-251 | 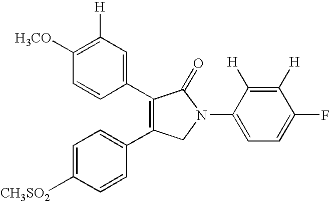 |
| B-252 | 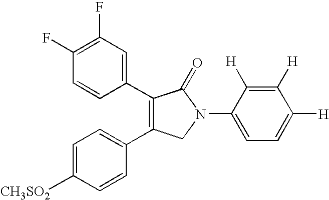 |

The cyclooxygenase-2 selective inhibitor employed in the present invention can exist in tautomeric, geometric or stereoisomeric forms. Generally speaking, suitable cyclooxygenase-2 selective inhibitors that are in tautomeric, geometric or stereoisomeric forms are those compounds that inhibit cyclooxygenase-2 activity by about 25%, more typically by about 50%, and even more typically, by about 75% or more when present at a concentration of 100 μM or less. The present invention contemplates all such compounds, including cis- and trans-geometric isomers, E- and Z-geometric isomers, R- and S-enantiomers, diastereomers, d-isomers, l-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

The cyclooxygenase-2 selective inhibitors utilized in the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethane sulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

The cyclooxygenase-2 selective inhibitors of the present invention can be formulated into pharmaceutical compositions and administered by a number of different means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the cyclooxygenase-2 selective inhibitor will vary depending upon the patient and the particular mode of administration. In general, the pharmaceutical compositions may contain a cyclooxygenase-2 selective inhibitor in the range of about 0.1 to 2000 mg, more typically, in the range of about 0.5 to 500 mg and still more typically, between about 1 and 200 mg. A daily dose of about 0.01 to 100 mg/kg body weight, or more typically, between about 0.1 and about 50 mg/kg body weight and even more typically, from about 1 to 20 mg/kg body weight, may be appropriate. The daily dose is generally administered in one to about four doses per day.

In one embodiment, when the cyclooxygenase-2 selective inhibitor comprises rofecoxib, it is typical that the amount used is within a range of from about 0.15 to about 1.0 mg/day·kg, and even more typically, from about 0.18 to about 0.4 mg/day·kg.

In still another embodiment, when the cyclooxygenase-2 selective inhibitor comprises etoricoxib, it is typical that the amount used is within a range of from about 0.5 to about 5 mg/day·kg, and even more typically, from about 0.8 to about 4 mg/day·kg.

Further, when the cyclooxygenase-2 selective inhibitor comprises celecoxib, it is typical that the amount used is within a range of from about 1 to about 20 mg/day·kg, even more typically, from about 1.4 to about 8.6 mg/day·kg, and yet more typically, from about 2 to about 3 mg/day·kg.

When the cyclooxygenase-2 selective inhibitor comprises valdecoxib, it is typical that the amount used is within a range of from about 0.1 to about 5 mg/day·kg, and even more typically, from about 0.8 to about 4 mg/day·kg.

In a further embodiment, when the cyclooxygenase-2 selective inhibitor comprises parecoxib, it is typical that the amount used is within a range of from about 0.1 to about 5 mg/day·kg, and even more typically, from about 1 to about 3 mg/day·kg.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

Anticonvulsant Agents

In addition to a cyclooxygenase-2 selective inhibitor, compositions and methods of the present invention may also comprise an anticonvulsant agent. A number of anticonvulsant agents useful in the treatment of CNS disorders or related conditions may be used in combination with a COX-2 selective inhibitor in the methods and compositions of the present invention.

Seizures or Related Disorders

Because of the wide variety of seizure types, each having a different etiology, not all anticonvulsant agents are effective at treating all types of seizures in all types of patients. In the practice of the present invention it is therefore desirable to individualize the use of an anticonvulsant agent based on the particular seizure and patient response. The methods of the present invention may thus be individualized to treat a number of different seizure or seizure disorder. By way of example, such seizures include but are not limited to, absence (petit mal) seizures, myoclonic seizures, clonic seizures, tonic seizures, tonic-clonic (grand mal) seizures, atonic seizures, simple partial (focal) seizures, complex partial seizures, and secondarily generalized tonic-clonic seizures. In one embodiment, the compositions and methods of the invention are used to treat epilepsy. The epilepsy may be either generalized or partial epilepsy.

Anticonvulsant agents useful for the treatment or control of seizures or seizure disorders can be used herein. A wide variety of anticonvulsant agents are known in the art. For extensive listings of anticonvulsants, see, e.g., Sweetman, S. C., ed., The Complete Drug Reference, 33^{rd} ed., Martindale Pharmaceutical Press (2002), pp. 338-71; and Mosby's DRUG Consult, Mosby, Inc. (2002), pp. 395-418. In one embodiment, the anticonvulsant agent is useful in the treatment or control of generalized tonic-clonic seizures or simple or complex partial seizures. In another embodiment, the anticonvulsant agent is useful in the treatment or control of absence seizures. In yet another embodiment, the anticonvulsant agent is useful in the treatment or control of seizures associated with the Lennox Gastaut syndrome. In a further embodiment, the anticonvulsant agent is useful in the treatment or control of status epilepticus. In still another embodiment, the anticonvulsant agent is useful in the treatment of epilepsy. The epilepsy may be either generalized or partial epilepsy.

Anticonvulsants may exert their anticonvulsive effect through a variety of complex mechanisms of action. Although the exact mechanism of action of some anticonvulsants is unknown, many anticonvulsants are thought to act through one or more of the following mechanisms, involving: 1) mediation of voltage-sensitive ion channels; 2) direct or indirect actions involving gamma aminobutyric acid (GABA) or the GABA_{A} receptor; and 3) inhibition of excitatory amino acids (EAA) such as glutamate, asparate, etc., by acting as EAA receptor antagonists.

As previously discussed, the blockage of voltage-sensitive ion channels may help to inhibit the repetitive neuronal firing associated with seizures. In particular, certain anticonvulsants are thought to play a role in the mediation of sodium and/or calcium channels. By blocking or inhibiting ion channel activity, anticonvulsants may help to stabilize neuronal firing by decreasing sodium and/or calcium conductance.

Thus, in one aspect of the invention, the anticonvulsant mediates the activity of sodium ion channels. Numerous examples of sodium channel blockers are known in the art, some of which are described below. Some non-limiting examples of anticonvulsants that are thought to mediate sodium ion channels include phenytoin, fosphenytoin, carbamazepine, valproic acid, felbamate, lamotrigine, topiramate, ethosuximide, and benzodiazepines, such as clonazepam and diazepam.

In another aspect of the invention, the anticonvulsant mediates the activity of calcium ion channels. Numerous examples of calcium channel blockers are known in the art, some of which are described below. Some non-limiting examples of anticonvulsants that are thought to mediate calcium ion channels include barbiturates, such as phenobarbital, mephobarbital, and metharbital; primidone; phenytoin; benzodiazepines, such as clonazepam and diazepam; ethosuximide; felbamate; lamotrigine; levetiracetam; and zonisamide.

Another mechanism by which some anticonvulsants may act is through direct or indirect actions involving gamma aminobutyric acid (GABA) or the GABA_{A} receptor. As previously discussed, GABA is an inhibitory neurotransmitter in the central nervous system (CNS) that interacts with the GABA_{A} receptor. Binding of GABA to GABA_{A} receptors results in the opening of a Cl⁻ channel. This allows chloride ions to flow into neurons, which in turn reduces the ability of neurons to depolarize to the threshold potential necessary to produce an action potential. It is thus desirable to increase GABA levels and/or regulate GABA/GABA_{A} receptor interactions when treating seizures.

Thus, in one embodiment, the anticonvulsant increases GABA levels and/or regulates GABA/GABA_{A} receptor interactions. Numerous examples of anticonvulsants that act on GABA or GABA_{A} receptors are known in the art, some of which are described below. One example of such an anticonvulsant is the benzodiazepines. Benzodiazepines modulate the binding of GABA to the GABA_{A} receptor by binding to the benzodiazepine receptor within the GABA-supramolecular complex. Barbiturates, such as pentobarbital and phenobarbital, as well as topiramate are also thought to enhance GABA/GABA_{A} receptor interactions. Other anticonvulsants, such as vigabatrin, valproic acid, gabapentin, and tiagabine are thought to exert an anticonvulsive effect by increasing GABA levels.

As previously discussed, anticonvulsants may also exert an anticonvulsive effect by inhibiting excitatory amino acids (EAA), such as glutamate, through their action as an excitatory amino acid receptor antagonist. There are several types of EAA receptors, including AMPA receptors, kainic acid receptors, and NMDA receptors. The NMDA receptor complex contains a number of distinct binding sites, including the glutamate-binding site, the glycine-binding site, and an NMDA binding site. Activation of a receptor by glutamate or glycine opens an ion channel, allowing an inflow of calcium and sodium ions, for example, which results in the firing of an action potential. The competitive binding of NMDA antagonists at a EAA recognition site can thus stop the effects of EAA.

Thus, in one embodiment, the anticonvulsant is an NMDA receptor antagonist. Numerous examples of anticonvulsants that act on NMDA receptors are known in the art, some of which are described below. Some non-limiting examples of anticonvulsants that are thought to act on the glycine or glutamate receptor of the NMDA complex include topiramate, gabapentin, and felbamate. In another embodiment, the anticonvulsant suppresses EAA excitation through interaction with non-NMDA EAA receptors. Examples of anticonvulsants that are thought to act on non-NMDA EAA receptors include, among others, barbiturates such as phenobarbital, mephobarbital, and metharbital.

Representative examples of these and other anticonvulsant agents suitable for use in the present invention include, among others, benzodiazepines such as clobazam, clonazepam, clorazepate, diazepam, nitrazepam, and lorazepam; succinimides such as ethosuximide, methsuximide, and phensuximide; barbiturates such as amobarbital, mephobarbital, metharbital, methylphenobarbital, phentobarbital, phenobarbital, primidone, and secobarbital; hydantoins such as ethotoin, fosphenytoin, fosphenytoin sodium, mephenyloin, and phenytoin; valproate (valproic acid and its salts and esters, such as valproic acid, valproate sodium, and divalproex); and diones, such as ethadione, paramethadione and trimethadione. Representative examples of other anticonvulsant agents suitable for use in the invention include, among others, carbamazepine, oxcarbazepine, primidone, acetazolamide, felbamate, gabapentin, igmesine, lamotrigine, levetiracetam, tiagabine, topiramate, vigabatrin, zonisamide, phenacemide, pheneturide, pregabalin, progabalin, ralitoline, remacemide hydrochloride, rufinamide, PD-165650, and PD-196860. In addition to these representative anticonvulsant agents, their prodrugs and pharmaceutically acceptable salts, esters, derivatives, analogs, and/or other related compounds effective in the treatment or control of seizures may also be used.

The anticonvulsant agents of the present invention can be administered alone or as an active ingredient in combination with pharmaceutically acceptable carriers, diluents, adjuvants and vehicles. They can be formulated into pharmaceutical compositions and administered to a subject by any suitable means generally known in the art that will deliver a therapeutically effective dose. For example, these pharmaceutical compositions may be given orally, parenterally, or rectally, or applied topically as an ointment, cream or powder. Oral, parenteral, or rectal administration of the compositions is generally preferred. The usual pharmaceutically acceptable carriers, diluents, adjuvants, vehicles, and additive materials may be used. These may be liquid or solid materials, which are otherwise inert or medically acceptable and are compatible with the active ingredients. Examples of such pharmaceutical adjuvants, diluents, and additive materials, as well as methods of administration include those discussed above for the preparation of pharmaceutical forms of the cyclooxygenase-2 selective inhibitor.

In treating the seizures or disorders described herein, occasionally it may be preferable to use two or more anticonvulsant agents in combination with each other and a COX-2 selective inhibitor in the composition of the present invention. When two or more anticonvulsant agents are used in combination in this way, any potential drug interactions between the anticonvulsant agents are considered in order to avoid reducing the effectiveness of the anticonvulsant agents or causing toxicity to the patient. Routine and standard procedures known in the art can be used to determine such interactions.

The anticonvulsant agents of the present invention are administered in such amount as will be therapeutically effective in the treatment or control of the CNS disorder or related condition being treated. It will be appreciated that the amount of active ingredients contained in an individual dose of each dosage form need not in itself constitute an effective amount, as the necessary effective amount could be reached by administration of a number of individual doses. Those skilled in the art will appreciate that the quantity of active anticonvulsant agent to be administered will vary depending upon the age, sex, and bodyweight of the subject to be treated, the type of seizure, disease, or syndrome to be treated, the particular method and scheduling of administration, and what other anticonvulsant agent, if any, is co-administered. Dosage amounts for an individual patient may thus be above or below the typical dosage ranges. Generally speaking, the anticonvulsant agent can be employed in any amount known to be effective at treating, preventing or controlling the disorder or condition being treated. The doses may be single doses or multiple doses per day, with the number of doses taken per day and the time allowed between doses varying depending on the individual needs of the patient. Optimization of treatment, including dosage amount, method and time of administration, and suitable anticonvulsant agents, is thus best determined by a skilled practitioner through close monitoring of patients on an individual basis.

As such, it should be noted that the dosage ranges herein, given by way of representative example, indicate only the typical dosage amounts administered to patients for that particular anticonvulsant agent for the treatment of seizures or epilepsy. Thus, they should not be construed as limiting amounts for the purpose of the present invention, as actual therapeutically effective dosage amounts for a patient may be more or less than the exemplary dosage range, depending on the individual needs of the patient, and the condition or disease being treated.

By way of example, in one embodiment, when the anticonvulsant agent is carbamazepine, it is typical that the amount used is within the range of approximately 400 to 2400 mg/day divided into 2 to 4 doses for adults and teenagers, with the initial dose typically being approximately 100 to 200 mg taken 1 to 2 times per day. For children, the amount used may be based on body weight, and is typically within the range of approximately 20 to 35 mg/kg/day in 2 to 4 divided doses, with the initial dose typically being approximately 5 to 10 mg/kg/day in two divided doses. Optimal dosage amounts will vary depending on the needs of the individual patient, but preferably will not exceed 1200 mg/day for patients over age 15, and 1000 mg/day for patients age 6 to 15. Dosages may be administered one to four times per day, depending on the needs of the individual patient and the dosage form. Typically, a low initial dose with a gradual increase to the minimum effective dose is advised.

By way of further example, when the anticonvulsant agent is valproic acid or its derivatives, dose may be based on body weight. Typically the amount used is within the range of approximately 10 to 60 mg/kg/day (or 375 to 4000 mg/day) given in 2 to 4 divided doses for adults and teenagers, with the initial dose typically being approximately 5 to 30 mg/kg/day (or 250 to 750 mg/day) given in 2 to 4 divided doses. The initial dose is then typically gradually increased by 5 to 10 mg/kg/week, as needed. For children, the amount used is typically within the range of approximately 15 to 60 mg/kg/day given in 2 to 4 divided doses, with the initial dose typically being approximately 15 to 20 mg/kg/day given in 2 to 4 divided doses.

In another embodiment, when the anticonvulsant agent is lamotrigine, the amount administered will vary depending on patient age and what other anticonvulsant agents are co-administered. The amount of lamotrigine administered is typically within the range of approximately 25 mg every other day to 700 mg/day when the patient is over 12 years of age, and approximately 0.15 to 15 mg/kg of body weight per day when the patient is age 2 to 12. The amount of lamotrigine administered to patients who are also taking enzyme inducing anticonvulsant agents (e.g. carbamazepine, phenobarbital, phenytoin, and/or primidone) but are not taking valproic acid (or derivatives), is preferably within the range of approximately 50 to 500 mg/day for patients over age 12, and approximately 0.6 to 15 mg/kg/day for patients age 2 to 12. The amount of lamotrigine administered to patients who are taking enzyme inducing anticonvulsant agents and valproic acid, is preferably within the range of approximately 25 mg every other day to 400 mg/day for patients over age 12, and approximately 0.15 to 5 mg/kg/day for patients age 2 to 12. Initial doses are usually within the lower end of the dosage ranges, and should be increased slowly, as needed, to avoid harmful side effects.

By way of further example, when the anticonvulsant agent is phenytoin or fosphenytoin, it is typical that the amount used is within the range of approximately 200 to 600 mg/day for adults and teenagers. The initial dose is typically within the range of approximately 3 to 5 mg/kg (200 to 400 mg/day) in 2 to 3 divided doses if given orally or approximately 10 to 20 mg/kg in one dose if given parenterally. For children, the amount used is based on body weight, and is typically within the range of approximately 4 to 8 mg/kg/day, with the initial dose typically being approximately 4 mg/kg/day and adjusted upwards if needed.

In a further example, when the anticonvulsant agent is gabapentin, it is typical that the amount used is within the range of approximately 600 to 4800 mg/day for adults and teenagers, with the initial dose typically being approximately 300 to 900 mg/day. For children, the amount used is based on body weight, and is typically within the range of approximately 25 to 60 mg/kg/day, with the initial dose typically being within the range of approximately 10 to 15 mg/kg/day and adjusted upwards if needed. The total daily dose is typically divided, and administered in 3 to 4 doses per day.

In another embodiment, when the anticonvulsant agent is topiramate, it is typical that the amount used is within the range of approximately 200 to 400 mg/day, with the initial dose typically being approximately 25 to 50 mg/day and slowly adjusted upwards as needed. Doses are typically divided and administered twice daily. For children, the amount used is based on body weight, and is typically within the range of approximately 6 to 9 mg/kg/day. The initial dose is typically approximately 0.5 to 1 mg/kg/day, and adjusted upwards in increments of approximately 0.5 to 1 mg/kg per dose as needed.

In a further example, when the anticonvulsant agent is ethosuximide, it is typical that the amount used is within the range of approximately 500 to 2000 mg/day for adults, with the initial dose typically being approximately 250 to 500 mg/day. The total daily dose may be administered in one daily dose, or divided and given in two doses per day. For children, the amount used may be based on body weight, and is typically within the range of approximately 15 to 40 mg/kg/day, with the initial dose typically being approximately 10 to 15 mg/kg/day, and adjusted upwards as needed.

In a further embodiment, when the anticonvulsant agent is tiagabin, it is typical that the amount used is within the range of approximately 32 to 64 mg/day for adults and teenagers, with the initial dose typically being approximately 4 mg/day, and slowly adjusted upwards as needed. For children, the amount used is based on body weight, and is typically approximately 1.0 mg/kg/day, with the initial dose typically being approximately 0.1 mg/kg/day and slowly adjusted upwards as needed. Doses are typically divided and given 2 to 4 times daily.

By way of further example, when the anticonvulsant agent is phenobarbital, it is typical that when administered orally, the amount used is within the range of approximately 30 to 320 mg/day for adults. When administered parenterally, the amount used is typically within the range of approximately 100 to 320 mg for adults. The dose may be repeated, but usually is not more than 600 mg/day. For children, the amount used is based on body weight, and is typically within the range of approximately 1 to 6 mg/kg/day when administered orally or parenterally. When administered parenterally, the initial dose is typically approximately 10 to 20 mg/kg/dose.

By way of further example, when the anticonvulsant agent is primidone, it is typical that the amount used is within the range of approximately 250 to 2000 mg/day in divided doses for adults and teenagers, with the initial dose typically being approximately 100 to 125 mg/day. For children, the amount used is based on body weight, and is typically within the range of approximately 10 to 50 mg/kg/day, with the initial dose typically being approximately 10 to 25 mg/kg/day.

In a further embodiment, when the anticonvulsant agent is clonazepam, it is typical that the amount used is within the range of approximately 0.5 to 20 mg/day in 2 to 4 divided doses for adults, with the initial dose typically being approximately 1.5 mg/day. For children, the amount used is based on body weight, and is typically within the range of approximately 0.1 to 0.3 mg/kg/day in 2 to 4 divided doses, with the initial dose typically being approximately 0.01 to 0.05 mg/kg/day.

In another embodiment, when the anticonvulsant agent is clorazepate, it is typical that the amount used is within the range of approximately 22.5-90 mg/day in 1 to 4 divided doses for adults and teenagers, with the initial dose typically being approximately 7.5 to 22.5 mg/day. For children, the amount used is typically within the range of approximately 7.5 to 60 mg/day in 1 to 4 divided doses, with the initial dose typically being approximately 3.75 to 7.5 mg/day.

In another embodiment, when the anticonvulsant agent is felbamate, it is typical that the amount used is within the range of approximately 600 to 3600 mg/day in 3 to 4 divided doses for adults and teenagers, with the initial dose typically being approximately 600 to 1200 mg/day adjusted upwards as needed. For children, the amount used is based upon body weight and is typically within the range of approximately 45 to 60 mg/kg/day in 3 to 4 divided doses, with the initial dose typically being approximately 15 mg/kg/day, adjusted upwards as needed.

By way of further example, when the anticonvulsant agent is zonisamide, it is typical that the amount used is within the range of approximately 100 to 800 mg/day in two divided doses for adults and teenagers, with the initial dose typically being approximately 100 mg/day in one dose and increased in 100 mg increments every 1 to 2 weeks as needed. For children, the amount used is based on body weight and is typically within the range of approximately 5 to 8 mg/kg/day in two divided doses, with the initial dose typically being approximately 1 to 2 mg/kg/day increased in increments of approximately 0.5 to 1.0 mg/kg every two weeks as needed.

By way of further example, when the anticonvulsant agent is levetiracetam, it is typical that the amount used is within the range of approximately 1000 to 3000 mg/day for adults, with the initial dose typically being approximately 1000 mg/day in two divided doses. For children, the amount used is based on body weight and is typically within the range of approximately 40 to 60 mg/kg/day, with the initial dose typically being approximately 20 mg/kg/day in two divided doses.

By way of further example, when the anticonvulsant agent is oxcarbazepine, it is typical that the amount used is with in the range of approximately 900 to 3000 mg/day, with the initial dose typically being approximately 400 to 600 mg/day in two divided doses. For children, the amount used is based on body weight and is typically within the range of approximately 8 to 60 mg/kg/day, with the initial dose typically being approximately 10 mg/kg/day in two divided doses.

Dosages for these and other suitable anticonvulsant agents may also be determined readily by those skilled in the art with guidance from Goodman & Goldman's The Pharmacological Basis of Therapeutics, 10^{th} ed. (2001); Sweetman, S. C., ed., The Complete Drug Reference, 33^{rd} ed., Martindale Pharmaceutical Press (2002), pp. 338-71; or Mosby's DRUG Consult, Mosby, Inc. (2002), pp. 395-418.

In some aspects, the invention provides treatment for subjects who are at risk of having a seizure. The invention embraces the treatment of subjects prior to a seizure and following a seizure to reduce the risk of future seizures, as well as treatment at the time of a seizure to reduce the duration or severity of the seizure.

As noted above, the dosage schedule and timing of administration of the anticonvulsant agent will vary considerably depending on the individual needs of the patient and the type of anticonvulsant agent used. Equally, the timing of the administration of the cyclooxygenase-2 selective inhibitor can also vary. For example, the cyclooxygenase-2 selective inhibitor can be administered beginning at a time prior to the onset of a seizure, at the time the seizure is occurring, or at a time after the seizure has occurred. Administration can be by a single dose, or more preferably the cyclooxygenase-2 selective inhibitor is given over an extended period of time as a prophylactic treatment. In one embodiment, administration is continued throughout the life of the subject to treat and control the onset of seizures associated with epilepsy.

The timing of the administration of the cyclooxygenase-2 selective inhibitor(s) in relation to the administration of the anticonvulsant agent or agents may also vary from subject to subject and depend upon the patient, the CNS disorder or related condition being treated, and the type of anticonvulsant agent being used. In one embodiment of the invention, the cyclooxygenase-2 selective inhibitor and anticonvulsant agent(s) may be administered substantially simultaneously, meaning that both agents may be administered to the subject in a single dosage, for example by mixing the agents and incorporating the mixture into a single capsule. Alternatively, the COX-2 inhibitor and anticonvulsant agent(s) may be administered substantially simultaneously by administration in separate dosages within a short time period. In another embodiment, the cyclooxygenase-2 selective inhibitor and anticonvulsant agent(s) may be administered sequentially, meaning that separate dosages, and possibly even separate dosage forms of the COX-2 inhibitor and anticonvulsant agent(s) may be administered at separate times, for example on a staggered schedule. Of course, it is also possible that the cyclooxygenase-2 selective inhibitor may be administered either more or less frequently than the anticonvulsant agent. Moreover, it will be apparent to those skilled in the art that it is possible, and perhaps desirable, to combine various times and methods of administration in the practice of the present invention. One skilled in the art can readily design suitable treatment regiments for a particular subject depending on the individual needs of the patient being treated.

Other CNS Disorders

Because of the diversity of agents that exhibit anticonvulsive effects and the complexity of their mechanisms of action, anticonvulsant agents may be beneficial in the treatment of a wide variety of CNS diseases or related conditions. However, just as not all anticonvulsant agents are effective in treating all types of seizures, not all anticonvulsant agents are effective in the treatment of all the CNS diseases and related conditions described herein. As such, the choice of anticonvulsant agent useful in the compositions and methods of the present invention is best determined by a skilled practitioner, based on the individual patient and the disease or condition being treated. In addition, the compositions of the invention may contain one or more anticonvulsant agent administered in combination with a cyclooxygenase-2 selective inhibitor or isomer, or pharmaceutically acceptable salt, ester, or prodrug thereof. When anticonvulsant agents are co-administered, drug interactions, such as those described above, are typically taken into account in determining the appropriate dosages.

In treating the CNS diseases and related conditions described herein, the compositions of the invention may be administered as monotherapy. Alternatively, it may be desirable to administer the compositions in combination with other drugs useful in the treatment of the specific CNS disease or related condition. Choice of treatment methods and modes of administration is best determined by a skilled practitioner based on the factors previously discussed.

The compositions and methods of the invention may be used in the treatment or control of a number of CNS disorders or related conditions on which anticonvulsant agents have a beneficial effect. Such disorders and conditions include psychiatric disorders, neurodegenerative disorders, withdrawal symptoms, and other disorders related to the action of voltage-sensitive ion channels, GABA and/or the GABA_{A} receptor, and glutamate receptors.

For example, the compositions and methods of the invention may be used in the treatment or control of mood disorders. Mood disorders include mania, depression, and various types of bipolar disorders (e.g. bipolar I disorder, bipolar II disorder, cyclothymia, and bipolar not-otherwise-specified (NOS)). A description of mood disorders as well as other mental disorders can be found in American Psychiatric Association Diagnostic and Statistical Manual of Mental Disorders, 4th ed. (DSM-IV).

A number of different anticonvulsant agents useful in the treatment or control of a mood disorder may be used in the present invention. Such anticonvulsants include, among others, valproate, carbamazepine, lamotrigine, gabapentin, tiagabine, topiramate, zonisamide, phenytoin, or oxcarbazepine. When the mood disorder is bipolar disorder, the anticonvulsant agent will generally be selected from the group consisting of carbamazepine, valproate, lamotrigine, gabapentin, tiagabine, or topiramate. When the mood disorder is mania or bipolar mania, the anticonvulsant agent is typically selected from the group consisting of carbamazepine, valproate, topiramate, or oxcarbamazepine, and is more typically carbamazepine or valproate. When the mood disorder is bipolar or unipolar depression, the anticonvulsant agent is generally selected from the group consisting of lamotrigine, topiramate, valproate, and gabapentin, and more typically is lamotrigine.

The compositions and methods of the present invention may also be used in the treatment or control of anxiety, anxiety disorders, and sleep disorders. Anxiety and agitation may be associated with an anxiety disorder, or may be the result of another psychic disorder, such as schizophrenia, major depression, and dementia, among others. There are several types of anxiety disorders, including, for example, panic disorders, phobias, obsessive-compulsive disorder (OCD), stress disorders (including both acute stress disorder and post-traumatic stress disorder (PTSD)), and generalized anxiety disorder (GAD). There are also a variety of different types of sleep disorders, many of which result in insomnia. Sleep disorders can be result from many conditions, diseases, or circumstances. Descriptions of anxiety and sleep disorders can be found in the DSM-IV.

A number of anticonvulsant agents useful in the treatment or control of anxiety, anxiety disorders, or sleep disorders may be employed in the invention. Such anticonvulsant agents include benzodiazepines, tiagabine, vigabatrin, carbamazepine, valproate, topiramate, and gabapentin. In one embodiment, the anticonvulsant agent is a benzodiazepine. In another embodiment, the benzodiazepine is selected from the group consisting of clorazepate, diazepam, and lorazepam.

The compositions and methods of the present invention may also be used in the treatment or control of neurodegenerative disorders. Excitotoxicity resulting from excessive stimulation of the NMDA receptors has been implicated in many CNS disorders, including neurodegenerative disorders. A number of neurodegenerative or related disorders that can be treated with an anticonvulsant may be treated by the compositions and methods of the present invention. Neurodegenerative or related disorders that may be treated by the present invention include, for example, Parkinson's disease, Huntington's disease, Alzheimer's disease, and amyotrophic lateral sclerosis (ALS), among others.

A number of anticonvulsant agents useful in the treatment or control of neurodegenerative disorders may be used in the present invention. Such anticonvulsant agents include gabapentin, carbamazepine, oxcarbazepine, topiramate, zonisamide, phenytoin, and lamotrigine, among others.

The compositions and methods of the present invention may also be used in the treatment or control of effects associated with the withdrawal from substances of abuse such as cocaine and other narcotics, alcohol, and benzodiazepines. Any anticonvulsant useful in reducing or eliminating symptoms associated with substance withdrawal may be used in the invention. Such anticonvulsant agents include, for example, gabapentin, pregabalin, vigabatrin, lamotrigine, carbamazepine, and valproate. Benzodiazepines may also be used in the treatment of symptoms associated with withdrawal, but since benzodiazepines have themselves been associated with tolerance and dependence, they should be used with caution.

Useful anticonvulsant agents in the treatment of obesity include, for example, topiramate, lamotrigine, valproate, carbamazepine, and felbamate. Preferably the anticonvulsant agent is topiramate.

Anticonvulsant agents such as carbamazepine, gabapentin, lamotrigine, phenobarbital, vigabatrin, benzodiazepines, clonazepam, valproic acid, topiramate, and tiagabine have also been used in the treatment of pain, migraine, and cluster headaches. Valproate in particular has been approved as a preventative treatment for migraine.

Other embodiments within the scope of the invention as described herein will be apparent to one skilled in the art from consideration of the specification or practice of the invention as disclosed herein. Because of the diversity of anticonvulsant agents and the CNS disorders or related conditions that they may be used to treat, it is intended that the specification be considered to be exemplary only, with the scope and spirit of the invention being indicated as described herein.

All references cited in this specification, including without limitation, all publications, patents, patent applications, texts, reports, books, journal articles, periodicals, and the like, are hereby incorporated by reference into this specification in their entireties.

As various changes could be made in the above methods and compositions without departing from the scope of the invention, it is intended that all matter contained in this application shall be interpreted as illustrative and not in a limiting sense.

Combination Therapies

Generally speaking, it is contemplated that the composition employed in the practice of the invention may include one or more of any of the cyclooxygenase-2 selective inhibitors detailed above in combination with one or more of any of the anticonvulsant agents detailed above. By way of a non-limiting example, Table 4a details a number of suitable combinations that are useful in the methods and compositions of the current invention. The combination may also include an isomer, a pharmaceutically acceptable salt, ester, or prodrug of any of the cyclooxygenase-2 selective inhibitors or anticonvulsant agents listed in Table 4a.

TABLE 4a

| Cyclooxygenase-2 Selective | |
|---|---|
| Inhibitor | anticonvulsant agent |
| a compound having formula I | carbamazepine |
| a compound having formula I | valproic acid |
| a compound having formula I | lamotrigine |
| a compound having formula I | phenytoin |
| a compound having formula I | gabapentin |
| a compound having formula I | topiramate |
| a compound having formula I | tiagabine |
| a compound having formula I | phenobarbital |
| a compound having formula I | primidone |
| a compound having formula I | clonazepam |
| a compound having formula II | carbamazepine |
| a compound having formula II | valproic acid |
| a compound having formula II | lamotrigine |
| a compound having formula II | phenytoin |
| a compound having formula II | gabapentin |
| a compound having formula II | topiramate |
| a compound having formula II | tiagabine |
| a compound having formula II | phenobarbital |
| a compound having formula II | primidone |
| a compound having formula II | clonazepam |
| a compound having formula III | carbamazepine |
| a compound having formula III | valproic acid |
| a compound having formula III | lamotrigine |
| a compound having formula III | phenytoin |
| a compound having formula III | gabapentin |
| a compound having formula III | topiramate |
| a compound having formula III | tiagabine |
| a compound having formula III | phenobarbital |
| a compound having formula III | primidone |
| a compound having formula III | clonazepam |
| a compound having formula IV | carbamazepine |
| a compound having formula IV | valproic acid |
| a compound having formula IV | lamotrigine |
| a compound having formula IV | phenytoin |
| a compound having formula IV | gabapentin |
| a compound having formula IV | topiramate |
| a compound having formula IV | tiagabine |
| a compound having formula IV | phenobarbital |
| a compound having formula IV | primidone |
| a compound having formula IV | clonazepam |
| a compound having formula V | carbamazepine |
| a compound having formula V | valproic acid |
| a compound having formula V | lamotrigine |
| a compound having formula V | phenytoin |
| a compound having formula V | gabapentin |
| a compound having formula V | topiramate |
| a compound having formula V | tiagabine |
| a compound having formula V | phenobarbital |
| a compound having formula V | primidone |
| a compound having formula V | clonazepam |

By way of further example, Table 4b details a number of suitable combinations that may be employed in the methods and compositions of the present invention. The combination may also include an isomer, a pharmaceutically acceptable salt, ester, or prodrug of any of the cyclooxygenase-2 selective inhibitors or anticonvulsant agents listed in Table 4b.

TABLE 4b

| Cyclooxygenase-2 Selective Inhibitor | anticonvulsant agent |
|---|---|
| a compound selected from the group consisting | carbamazepine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252 | |
| a compound selected from the group consisting | valproic acid |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252 | |
| a compound selected from the group consisting | lamotrigine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252 | |
| a compound selected from the group consisting | phenytoin |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252 | |
| a compound selected from the group consisting | gabapentin |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252 | |
| a compound selected from the group consisting | topiramate |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252 | |
| a compound selected from the group consisting | tiagabine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252 | |
| a compound selected from the group consisting | phenobarbital |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252 | |
| a compound selected from the group consisting | primidone |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252 | |
| a compound selected from the group consisting | clonazepam |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252 | |

By way of yet further example, Table 4c details additional suitable combinations that may be employed in the methods and compositions of the current invention. The combination may also include an isomer, a pharmaceutically acceptable salt, ester, or prodrug of any of the cyclooxygenase-2 selective inhibitors or anticonvulsant agents listed in Table 4c.

TABLE 4c

| Cyclooxygenase-2 Selective | |
|---|---|
| Inhibitor | anticonvulsant agent |
| celecoxib | carbamazepine |
| celecoxib | valproic acid |
| celecoxib | lamotrigine |
| celecoxib | phenytoin |
| celecoxib | gabapentin |
| celecoxib | topiramate |
| celecoxib | tiagabine |
| celecoxib | phenobarbital |
| celecoxib | primidone |
| celecoxib | clonazepam |
| deracoxib | carbamazepine |
| deracoxib | valproic acid |
| deracoxib | lamotrigine |
| deracoxib | phenytoin |
| deracoxib | gabapentin |
| deracoxib | topiramate |
| deracoxib | tiagabine |
| deracoxib | phenobarbital |
| deracoxib | primidone |
| deracoxib | clonazepam |
| valdecoxib | carbamazepine |
| valdecoxib | valproic acid |
| valdecoxib | lamotrigine |
| valdecoxib | phenytoin |
| valdecoxib | gabapentin |
| valdecoxib | topiramate |
| valdecoxib | tiagabine |
| valdecoxib | phenobarbital |
| valdecoxib | primidone |
| valdecoxib | clonazepam |
| rofecoxib | carbamazepine |
| rofecoxib | valproic acid |
| rofecoxib | lamotrigine |
| rofecoxib | phenytoin |
| rofecoxib | gabapentin |
| rofecoxib | topiramate |
| rofecoxib | tiagabine |
| rofecoxib | phenobarbital |
| rofecoxib | primidone |
| rofecoxib | clonazepam |
| etoricoxib | carbamazepine |
| etoricoxib | valproic acid |
| etoricoxib | lamotrigine |
| etoricoxib | phenytoin |
| etoricoxib | gabapentin |
| etoricoxib | topiramate |
| etoricoxib | tiagabine |
| etoricoxib | phenobarbital |
| etoricoxib | primidone |
| etoricoxib | clonazepam |
| meloxicam | carbamazepine |
| meloxicam | valproic acid |
| meloxicam | lamotrigine |
| meloxicam | phenytoin |
| meloxicam | gabapentin |
| meloxicam | topiramate |
| meloxicam | tiagabine |
| meloxicam | phenobarbital |
| meloxicam | primidone |
| meloxicam | clonazepam |
| parecoxib | carbamazepine |
| parecoxib | valproic acid |
| parecoxib | lamotrigine |
| parecoxib | phenytoin |
| parecoxib | gabapentin |
| parecoxib | topiramate |
| parecoxib | tiagabine |
| parecoxib | phenobarbital |
| parecoxib | primidone |
| parecoxib | clonazepam |
| 4-(4-cyclohexyl-2-methyloxazol-5- | carbamazepine |
| yl)-2-fluorobenzenesulfonamide | |
| 4-(4-cyclohexyl-2-methyloxazol-5- | valproic acid |
| yl)-2-fluorobenzenesulfonamide | |
| 4-(4-cyclohexyl-2-methyloxazol-5- | lamotrigine |
| yl)-2-fluorobenzenesulfonamide | |
| 4-(4-cyclohexyl-2-methyloxazol-5- | phenytoin |
| yl)-2-fluorobenzenesulfonamide | |
| 4-(4-cyclohexyl-2-methyloxazol-5- | gabapentin |
| yl)-2-fluorobenzenesulfonamide | |
| 4-(4-cyclohexyl-2-methyloxazol-5- | topiramate |
| yl)-2-fluorobenzenesulfonamide | |
| 4-(4-cyclohexyl-2-methyloxazol-5- | tiagabine |
| yl)-2-fluorobenzenesulfonamide | |
| 4-(4-cyclohexyl-2-methyloxazol-5- | phenobarbital |
| yl)-2-fluorobenzenesulfonamide | |
| 4-(4-cyclohexyl-2-methyloxazol-5- | primidone |
| yl)-2-fluorobenzenesulfonamide | |
| 4-(4-cyclohexyl-2-methyloxazol-5- | clonazepam |
| yl)-2-fluorobenzenesulfonamide | |
| 2-(3,5-difluorophenyl)-3-(4- | carbamazepine |
| (methylsulfonyl)phenyl)-2- | |
| cyclopenten-1-one | |
| 2-(3,5-difluorophenyl)-3-(4- | valproic acid |
| (methylsulfonyl)phenyl)-2- | |
| cyclopenten-1-one | |
| 2-(3,5-difluorophenyl)-3-(4- | lamotrigine |
| (methylsulfonyl)phenyl)-2- | |
| cyclopenten-1-one | |
| 2-(3,5-difluorophenyl)-3-(4- | phenytoin |
| (methylsulfonyl)phenyl)-2- | |
| cyclopenten-1-one | |
| 2-(3,5-difluorophenyl)-3-(4- | gabapentin |
| (methylsulfonyl)phenyl)-2- | |
| cyclopenten-1-one | |
| 2-(3,5-difluorophenyl)-3-(4- | topiramate |
| (methylsulfonyl)phenyl)-2- | |
| cyclopenten-1-one | |
| 2-(3,5-difluorophenyl)-3-(4- | tiagabine |
| (methylsulfonyl)phenyl)-2- | |
| cyclopenten-1-one | |
| 2-(3,5-difluorophenyl)-3-(4- | phenobarbital |
| (methylsulfonyl)phenyl)-2- | |
| cyclopenten-1-one | |
| 2-(3,5-difluorophenyl)-3-(4- | primidone |
| (methylsulfonyl)phenyl)-2- | |
| cyclopenten-1-one | |
| 2-(3,5-difluorophenyl)-3-(4- | clonazepam |
| (methylsulfonyl)phenyl)-2- | |
| cyclopenten-1-one | |
| N-[2-(cyclohexyloxy)-4-nitrophenyl] | carbamazepine |
| methanesulfonamide | |
| N-[2-(cyclohexyloxy)-4-nitrophenyl] | valproic acid |
| methanesulfonamide | |
| N-[2-(cyclohexyloxy)-4- | lamotrigine |
| nitrophenyl]methanesulfonamide | |
| N-[2-(cyclohexyloxy)-4-nitrophenyl] | phenytoin |
| methanesulfonamide | |
| N-[2-(cyclohexyloxy)-4-nitrophenyl] | gabapentin |
| methanesulfonamide | |
| N-[2-(cyclohexyloxy)-4-nitrophenyl] | topiramate |
| methanesulfonamide | |
| N-[2-(cyclohexyloxy)-4-nitrophenyl] | tiagabine |
| methanesulfonamide | |
| N-[2-(cyclohexyloxy)-4-nitrophenyl] | phenobarbital |
| methanesulfonamide | |
| N-[2-(cyclohexyloxy)-4-nitrophenyl] | primidone |
| methanesulfonamide | |
| N-[2-(cyclohexyloxy)-4-nitrophenyl] | clonazepam |
| methanesulfonamide | |
| 2-(3,4-difluorophenyl)-4-(3-hydroxy- | carbamazepine |
| 3-methylbutoxy)-5-[4- | |
| (methylsulfonyl)phenyl]-3(2H)- | |
| pyridazinone | |
| 2-(3,4-difluorophenyl)-4-(3-hydroxy- | valproic acid |
| 3-methylbutoxy)-5-[4- | |
| (methylsulfonyl)phenyl]-3(2H)- | |
| pyridazinone | |
| 2-(3,4-difluorophenyl)-4-(3-hydroxy- | lamotrigine |
| 3-methylbutoxy)-5-[4- | |
| (methylsulfonyl)phenyl]-3(2H)- | |
| pyridazinone | |
| 2-(3,4-difluorophenyl)-4-(3-hydroxy- | phenytoin |
| 3-methylbutoxy)-5-[4- | |
| (methylsulfonyl)phenyl]-3(2H)- | |
| pyridazinone | |
| 2-(3,4-difluorophenyl)-4-(3-hydroxy- | gabapentin |
| 3-methylbutoxy)-5-[4- | |
| (methylsulfonyl)phenyl]-3(2H)- | |
| pyridazinone | |
| 2-(3,4-difluorophenyl)-4-(3-hydroxy- | topiramate |
| 3-methylbutoxy)-5-[4- | |
| (methylsulfonyl)phenyl]-3(2H)- | |
| pyridazinone | |
| 2-(3,4-difluorophenyl)-4-(3-hydroxy- | tiagabine |
| 3-methylbutoxy)-5-[4- | |
| (methylsulfonyl)phenyl]-3(2H)- | |
| pyridazinone | |
| 2-(3,4-difluorophenyl)-4-(3-hydroxy- | phenobarbital |
| 3-methylbutoxy)-5-[4- | |
| (methylsulfonyl)phenyl]-3(2H)- | |
| pyridazinone | |
| 2-(3,4-difluorophenyl)-4-(3-hydroxy- | primidone |
| 3-methylbutoxy)-5-[4- | |
| (methylsulfonyl)phenyl]-3(2H)- | |
| pyridazinone | |
| 2-(3,4-difluorophenyl)-4-(3-hydroxy- | clonazepam |
| 3-methylbutoxy)-5-[4- | |
| (methylsulfonyl)phenyl]-3(2H)- | |
| pyridazinone | |
| 2-[(2,4-dichloro-6-methylphenyl) | carbamazepine |
| amino]-5-ethyl-benzeneacetic acid | |
| 2-[(2,4-dichloro-6-methylphenyl) | valproic acid |
| amino]-5-ethyl-benzeneacetic acid | |
| 2-[(2,4-dichloro-6-methylphenyl) | lamotrigine |
| amino]-5-ethyl-benzeneacetic acid | |
| 2-[(2,4-dichloro-6-methylphenyl) | phenytoin |
| amino]-5-ethyl-benzeneacetic acid | |
| 2-[(2,4-dichloro-6-methylphenyl) | gabapentin |
| amino]-5-ethyl-benzeneacetic acid | |
| 2-[(2,4-dichloro-6-methylphenyl) | topiramate |
| amino]-5-ethyl-benzeneacetic acid | |
| 2-[(2,4-dichloro-6-methylphenyl) | tiagabine |
| amino]-5-ethyl-benzeneacetic acid | |
| 2-[(2,4-dichloro-6-methylphenyl) | phenobarbital |
| amino]-5-ethyl-benzeneacetic acid | |
| 2-[(2,4-dichloro-6-methylphenyl) | primidone |
| amino]-5-ethyl-benzeneacetic acid | |
| 2-[(2,4-dichloro-6-methylphenyl) | clonazepam |
| amino]-5-ethyl-benzeneacetic acid | |
| (3Z)-3-[(4-chlorophenyl)[4- | carbamazepine |
| (methylsulfonyl)phenyl]methylene]dihydro- | |
| 2(3H)-furanone | |
| (3Z)-3-[(4-chlorophenyl)[4- | valproic acid |
| (methylsulfonyl)phenyl]methylene]dihydro- | |
| 2(3H)-furanone | |
| (3Z)-3-[(4-chlorophenyl)[4- | lamotrigine |
| (methylsulfonyl)phenyl]methylene]dihydro- | |
| 2(3H)-furanone | |
| (3Z)-3-[(4-chlorophenyl)[4- | phenytoin |
| (methylsulfonyl)phenyl]methylene]dihydro- | |
| 2(3H)-furanone | |
| (3Z)-3-[(4-chlorophenyl)[4- | gabapentin |
| (methylsulfonyl)phenyl]methylene]dihydro- | |
| 2(3H)-furanone | |
| (3Z)-3-[(4-chlorophenyl)[4- | topiramate |
| (methylsulfonyl)phenyl]methylene]dihydro- | |
| 2(3H)-furanone | |
| (3Z)-3-[(4-chlorophenyl)[4- | tiagabine |
| (methylsulfonyl)phenyl]methylene]dihydro- | |
| 2(3H)-furanone | |
| (3Z)-3-[(4-chlorophenyl)[4- | phenobarbital |
| (methylsulfonyl)phenyl]methylene]dihydro- | |
| 2(3H)-furanone | |
| (3Z)-3-[(4-chlorophenyl)[4- | primidone |
| (methylsulfonyl)phenyl]methylene]dihydro- | |
| 2(3H)-furanone | |
| (3Z)-3-[(4-chlorophenyl)[4- | clonazepam |
| (methylsulfonyl)phenyl]methylene]dihydro- | |
| 2(3H)-furanone | |
| (S)-6,8-dichloro-2-(trifluoromethyl)- | carbamazepine |
| 2H-1-benzopyran-3-carboxylic acid | |
| (S)-6,8-dichloro-2-(trifluoromethyl)- | valproic acid |
| 2H-1-benzopyran-3-carboxylic acid | |
| (S)-6,8-dichloro-2-(trifluoromethyl)- | lamotrigine |
| 2H-1-benzopyran-3-carboxylic acid | |
| (S)-6,8-dichloro-2-(trifluoromethyl)- | phenytoin |
| 2H-1-benzopyran-3-carboxylic acid | |
| (S)-6,8-dichloro-2-(trifluoromethyl)- | gabapentin |
| 2H-1-benzopyran-3-carboxylic acid | |
| (S)-6,8-dichloro-2-(trifluoromethyl)- | topiramate |
| 2H-1-benzopyran-3-carboxylic acid | |
| (S)-6,8-dichloro-2-(trifluoromethyl)- | tiagabine |
| 2H-1-benzopyran-3-carboxylic acid | |
| (S)-6,8-dichloro-2-(trifluoromethyl)- | phenobarbital |
| 2H-1-benzopyran-3-carboxylic acid | |
| (S)-6,8-dichloro-2-(trifluoromethyl)- | primidone |
| 2H-1-benzopyran-3-carboxylic acid | |
| (S)-6,8-dichloro-2-(trifluoromethyl)- | clonazepam |
| 2H-1-benzopyran-3-carboxylic acid | |
| lumiracoxib | carbamazepine |
| lumiracoxib | valproic acid |
| lumiracoxib | lamotrigine |
| lumiracoxib | phenytoin |
| lumiracoxib | gabapentin |
| lumiracoxib | topiramate |
| lumiracoxib | tiagabine |
| lumiracoxib | phenobarbital |
| lumiracoxib | primidone |
| lumiracoxib | clonazepam |

### EXAMPLES

A combination therapy of a COX-2 selective inhibitor and an anticonvulsant agent for the treatment or prevention of a vaso-occlusive event or a related disorder in a subject can be evaluated as described in the following tests detailed below.

A particular combination therapy comprising an anticonvulsant agent and a COX-2 inhibitor can be evaluated in comparison to a control treatment such as a placebo treatment, administration of a COX-2 inhibitor only or administration of an anticonvulsant agent only. By way of example, a combination therapy may contain any of the anticonvulsant agents and any of the COX-2 inhibitors detailed in the present invention, including the combinations set forth in Tables 4a, 4b, or 4c. The dosages of an anticonvulsant agent and a COX-2 inhibitor in a particular therapeutic combination may be readily determined by a skilled artisan conducting the study. The length of the study treatment will vary on a particular study and can also be determined by one of ordinary skill in the art. By way of example, the combination therapy may be administered for 4 weeks. The anticonvulsant agent and COX-2 inhibitor can be administered by any route as described herein, but are preferably administered orally for human subjects.

### Example 1

### Evaluation of COX-1 and COX-2 Activity In Vitro

The COX-2 inhibitors suitable for use in this invention exhibit selective inhibition of COX-2 over COX-1 when tested in vitro according to the following activity assays.

Preparation of Recombinant COX Baculoviruses

Recombinant COX-1 and COX-2 are prepared as described by Gierse et al, [J. Biochem., 305, 479-84 (1995)]. A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×10⁸) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (10⁷-10⁸ pfu/mL) stocks of virus are prepared. For large scale production, SF9 insect cells are infected in 10 liter fermentors (0.5×106/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for COX activity.

Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after ten minutes at 37° C. by transferring 40 μl of reaction mix into 160 μl ELISA buffer and 25 μM indomethacin. The PGE2 formed is measured by standard ELISA technology (Cayman Chemical).

Fast Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds are pre-incubated with the enzyme for 10 minutes at 25° C. prior to the addition of arachidonic acid. Any reaction between the arachidonic acid and the enzyme is stopped after two minutes at 37° C. by transferring 40 μl of reaction mix into 160 μl ELISA buffer and 25 μM indomethacin. Indomethacin, a non-selective COX-2/COX-1 inhibitor, may be utilized as a positive control. The PGE₂ formed is typically measured by standard ELISA technology utilizing a PGE2 specific antibody, available from a number of commercial sources.

Each compound to be tested may be individually dissolved in 2 ml of dimethyl sulfoxide (DMSO) for bioassay testing to determine the COX-1 and COX-2 inhibitory effects of each particular compound. Potency is typically expressed by the IC₅₀ value expressed as g compound/ml solvent resulting in a 50% inhibition of PGE2 production. Selective inhibition of COX-2 may be determined by the IC₅₀ ratio of COX-1/COX-2.

By way of example, a primary screen may be performed in order to determine particular compounds that inhibit COX-2 at a concentration of 10 ug/ml. The compound may then be subjected to a confirmation assay to determine the extent of COX-2 inhibition at three different concentrations (e.g., 10 ug/ml, 3.3 ug/ml and 1.1 ug/ml). After this screen, compounds can then be tested for their ability to inhibit COX-1 at a concentration of 10 ug/ml. With this assay, the percentage of COX inhibition compared to control can be determined, with a higher percentage indicating a greater degree of COX inhibition. In addition, the IC₅₀ value for COX-1 and COX-2 can also be determined for the tested compound. The selectivity for each compound may then be determined by the IC₅₀ ratio of COX-1/COX-2, as set-forth above.

### Example 2

### Methods for Measuring Platelet Aggregation and Platelet Activation Markers

The following studies can be performed in human subjects or laboratory animal models, such as mice. Prior to the initiation of a clinical study involving human subjects, the study should be approved by the appropriate Human Subjects Committee and subjects should be informed about the study and give written consent prior to participation.

Platelet activation can be determined by a number of tests available in the art. Several such tests are described below. In order to determine the effectiveness of the treatment, the state of platelet activation is evaluated at several time points during the study, such as before administering the combination treatment and once a week during treatment. The exemplary procedures for blood sampling and the analyses that can be used to monitor platelet aggregation are listed below.

Platelet Aggregation Study

Blood samples are collected from an antecubital vein via a 19-gauge needle into two plastic tubes. Each sample of free flowing blood is collected through a fresh venipuncture site distal to any intravenous catheters using a needle and Vacutainer hood into 7 cc vacutainer tubes (one with CTAD (dipyridamole), and the other with 3.8% trisodium citrate). If blood is collected simultaneously for any other studies, it is preferable that the platelet sample be obtained second or third, but not first. If only the platelet sample is collected, the initial 2-3 cc of blood is discharged and then the vacutainer tube is filled. The venipuncture is adequate if the tube fills within 15 seconds. All collections are performed by trained personnel.

After the blood samples for each subject have been collected into two Vacutainer tubes, they are immediately, but gently, inverted 3 to 5 times to ensure complete mixing of the anticoagulant. Tubes are not shaken. The Vacutainer tubes are filled to capacity, since excess anticoagulant can alter platelet function. Attention is paid to minimizing turbulence whenever possible. Small steps, such as slanting the needle in the Vacutainer to have the blood run down the side of tube instead of shooting all the way to the bottom, can result in significant improvement. These tubes are kept at room temperature and transferred directly to the laboratory personnel responsible for preparing the samples. The Vacutainer tubes are not chilled at any time.

Trisodium citrate (3.8%) and whole blood is immediately mixed in a 1:9 ratio, and then centrifuged at 1200 g for 2.5 minutes, to obtain platelet-rich plasma (PRP), which is kept at room temperature for use within 1 hour for platelet aggregation studies. Platelet count is determined in each PRP sample with a Coulter Counter ZM (Coulter Co., Hialeah, Fla.). Platelet numbers are adjusted to 3.50×10⁸/ml for aggregation with homologous platelet-poor plasma. PRP and whole blood aggregation tests are performed simultaneously. Whole blood is diluted 1:1 with the 0.5 ml PBS, and then swirled gently to mix. The cuvette with the stirring bar is placed in the incubation well and allowed to warm to 37° C. for 5 minutes. Then the samples are transferred to the assay well. An electrode is placed in the sample cuvette. Platelet aggregation is stimulated with 5 μM ADP, 1 μg/ml collagen, and 0.75 mM arachidonic acid. All agonists are obtained, e.g., from Chronolog Corporation (Hawertown, Pa.). Platelet aggregation studies are performed using a Chrono-Log Whole Blood Lumi-Aggregometer (model 560-Ca). Platelet aggregability is expressed as the percentage of light transmittance change from baseline using platelet-poor plasma as a reference at the end of recording time for plasma samples, or as a change in electrical impedance for whole blood samples. Aggregation curves are recorded for 4 minutes and analyzed according to internationally established standards using Aggrolink® software.

Aggregation curves of subjects receiving a combination therapy containing an anticonvulsant agent and a COX-2 inhibitor can then be compared to the aggregation curves of subjects receiving a control treatment in order to determine the efficacy of said combination therapy.

Washed Platelets Flow Cytometry

Venous blood (8 ml) is collected in a plastic tube containing 2 ml of acid-citrate-dextrose (ACD) (7.3 g citric acid, 22.0 g sodium citrate × 2H₂O and 24.5 glucose in 1000 ml distilled water) and mixed well. The blood-ACD mixture is centrifuged at 1000 r.p.m. for 10 minutes at room temperature. The upper ⅔ of the platelet-rich plasma (PRP) is then collected and adjusted to pH=6.5 by adding ACD. The PRP is then centrifuged at 3000 r.p.m. for 10 minutes. The supernatant is removed and the platelet pellet is gently resuspended in 4 cc of the washing buffer (10 mM Tris/HCl, 0.15 M NaCl, 20 mM EDTA, pH=7.4). Platelets are washed in the washing buffer, and in TBS (10 mM Tris, 0.15 M NaCl, pH=7.4). All cells are then divided into the appropriate number of tubes. By way of example, if 9 different surface markers are evaluated, as described herein, then the cells should be divided into ten tubes, such that nine tubes containing washed platelets are incubated with 5 μl fluorescein isothiocyanate (FITC)-conjugated antibodies in the dark at +4° C. for 30 minutes, and one tube remains unstained and serves as a negative control. Surface antigen expression is measured with monoclonal murine anti-human antibodies, such as CD9 (p24); CD41a (IIb/IIIa, aIIbb3); CD42b (Ib); CD61 (IIIa) (DAKO Corporation, Carpinteria, Calif.); CD49b (VLA-2, or a2b1); CD62p (P-selectin); CD31 (PECAM-1); CD 41b (IIb); and CD51/CD61 (vitronectin receptor, avb3) (PharMingen, San Diego Calif.), as the expression of these antigens on the cells is associated with platelet activation. After incubation, the cells are washed with TBS and resuspended in 0.25 ml of 1% paraformaldehyde. Samples are stored in the refrigerator at +4° C., and analyzed on a Becton Dickinson FACScan flow cytometer with laser output of 15 mw, excitation at 488 nm, and emission detection at 530+−30 nm. The data can be collected and stored in list mode, and then analyzed using CELLQuest® software. FACS procedures are described in detail in, e.g., Gurbel, P. A. et al., J Amer Coll Cardiol 31: 1466-1473 (1998); Serebruany, V. L. et al., Am Heart J 136: 398-405 (1998); Gurbel, P. A. et al., Coron Artery Dis 9: 451-456 (1998) and Serebruany, V. L. et al., Arterioscl Thromb Vasc Biol 19:153-158 (1999).

The antibody staining of platelets isolated from subjects receiving a combination therapy can then be compared to the staining of platelets isolated from subjects receiving a control treatment in order to determine the effect of the combination therapy on platelets.

Whole Blood Flow Cytometry

Four cc of blood is collected in a tube, containing 2 cc of acid-citrate-dextrose (ACD, see previous example) and mixed well. The buffer, TBS (10 mM Tris, 0.15 M NaCl, pH 7.4) and the following fluorescein isothiocyanate (FITC) conjugated monoclonal antibodies (PharMingen, San Diego, Calif., USA, and DAKO, Calif., USA) are removed from a refrigerator and allowed to warm at room temperature (RT) prior to their use. The non-limiting examples of antibodies that can be used include CD41 (IIIb/IIIa), CD31 (PECAM-1), CD62p (P-selectin), and CD51/61 (Vitronectin receptor). For each subject, six amber tubes (1.25 ml) are one Eppendorf tube (1.5 ml) are obtained and marked appropriately. 450 μl of TBS buffer is pipetted to the labeled Eppendorf tube. A patient's whole blood tube is inverted gently twice to mix, and 50 μl of whole blood is pipetted to the appropriately labeled Eppendorf tube. The Eppendorf tube is capped and the diluted whole blood is mixed by inverting the Eppendorf tube gently two times, followed by pipetting 50 μl of diluted whole blood to each amber tube. 5 μl of appropriate antibody is pipetted to the bottom of the corresponding amber tube. The tubes are covered with aluminum foil and incubated at 4° C. for 30 minutes. After incubation, 400 μl of 2% buffered paraformaldehyde is added. The amber tubes are closed with a lid tightly and stored in a refrigerator at 4° C. until the flow cytometric analysis. The samples are analyzed on a Becton Dickinson FACScan flow cytometer. These data are collected in list mode files and then analyzed. As mentioned in (B.), the antibody staining of platelets isolated from subjects receiving a combination therapy can then be compared to the staining of platelets isolated from subjects receiving a control treatment.

ELISA

Enzyme-linked immunosorbent assays (ELISA) are used according to standard techniques and as described herein. Eicosanoid metabolites may be used to determine platelet aggregation. The metabolites are analyzed due to the fact that eicosanoids have a short half-life under physiological conditions. Thromboxane B2 (TXB₂), the stable breakdown product of thromboxane A₂ and 6keto-PGF₁ alpha, the stable degradation product of prostacyclin may be tested. Thromboxane B2 is a stable hydrolysis product of TXA₂ and is produced following platelet aggregation induced by a variety of agents, such as thrombin and collagen. 6keto-prostaglandin F₁ alpha is a stable hydrolyzed product of unstable PGI₂ (prostacyclin). Prostacyclin inhibits platelet aggregation and induces vasodilation. Thus, quantitation of prostacyclin production can be made by determining the level of 6keto-PGF₁. The metabolites may be measured in the platelet poor plasma (PPP), which is kept at −4° C. Also, plasma samples may also be extracted with ethanol and then stored at −80° C. before final prostaglandin determination, using, e.g., TiterZymes® enzyme immunoassays according to standard techniques (PerSeptive Diagnostics, Inc., Cambridge, Mass., USA). ELISA kits for measuring TXB₂ and 6keto-PGF₁ are also commercially available.

The amounts of TXB₂ and 6keto-PGF₁ in plasma of subjects receiving a combination therapy and subjects receiving a control therapy can be compared to determine the efficacy of the combination treatment.

Closure Time Measured with the Dade Behring Platelet Function Analyzer, PFA-100®

PFA-100® can be used as an in vitro system for the detection of platelet dysfunction. It provides a quantitative measure of platelet function in anticoagulated whole blood. The system comprises a microprocessor-controlled instrument and a disposable test cartridge containing a biologically active membrane. The instrument aspirates a blood sample under constant vacuum from the sample reservoir through a capillary and a microscopic aperture cut into the membrane. The membrane is coated with collagen and epinephrine or adenosine 5′-diphosphate. The presence of these biochemical stimuli, and the high shear rates generated under the standardized flow conditions, result in platelet attachment, activation, and aggregation, slowly building a stable platelet plug at the aperture. The time required to obtain full occlusion of the aperture is reported as the “closure time,” which normally ranges from one to three minutes.

The membrane in the PFA-100® test cartridge serves as a support matrix for the biological components and allows placement of the aperture. The membrane is a standard nitrocellulose filtration membrane with an average pore size of 0.45 μm. The blood entry side of the membrane was coated with 2 μg of fibrillar Type I equine tendon collagen and 10 μg of epinephrine bitartrate or 50 μg of adenosine 5′-diphosphate (ADP). These agents provide controlled stimulation to the platelets as the blood sample passes through the aperture. The collagen surface also served as a well-defined matrix for platelet deposition and attachment.

The principle of the PFA-100® test is very similar to that described by Kratzer and Born (Kratzer, et al., Haemostasis 15: 357-362 (1985)). The test utilizes whole blood samples collected in 3.8% of 3.2% sodium citrate anticoagulant. The blood sample is aspirated through the capillary into the cup where it comes in contact with the coated membrane, and then passes through the aperture. In response to the stimulation by collagen and epinephrine or ADP present in the coating, and the shear stresses at the aperture, platelets adhere and aggregate on the collagen surface starting at the area surrounding the aperture. During the course of the measurement, a stable platelet plug forms that ultimately occludes the aperture. The time required to obtain full occlusion of the aperture is defined as the “closure time” and is indicative of the platelet function in the sample. Accordingly, “closure times” can be compared between subjects receiving a combination therapy and the ones receiving a control therapy in order to evaluate the efficacy of the combination treatment. By way of example, a combination therapy may contain 2-arachidonylglycerol and celecoxib, N-arachidonyl and valdecoxib, 5-(4-chlorophenyl)-1-(2,4-dichlorophenyl)-4-methyl-n-1-piperidinyl-1 h-pyrazole-3-caroxamide (SR 141716A) and rofecoxib, or [6-methoxy-2-(4-methoxyphenyl)benzo[b]fu ran-3-yl](4-cyanophenyl)methanone (LY 320135) and celecoxib. It should be noted that these are only several examples, and that any of the anticonvulsant agents in combination with any of the Cox-2 inhibitors of the present invention may be tested as a combination therapy. The dosages of the anticonvulsant agent and Cox-2 inhibitor in a particular therapeutic combination may be readily determined by a skilled artisan conducting the study. The length of the study treatment will vary on a particular study and can also be determined by one of ordinary skill in the art. By way of example, the combination therapy may be administered for 12 weeks. The anticonvulsant agent and Cox-2 inhibitor can be administered by any route as described herein, but are preferably administered orally or intravenously for human subjects.

1. A method for treating a central nervous system disorder or related condition, the method comprising:
(a) diagnosing a subject in need of treatment for a central nervous system disorder or related condition; and
(b) administering to the subject a cyclooxygenase-2 selective inhibitor or an isomer, a pharmaceutically acceptable salt, ester, or prodrug thereof and an anticonvulsant agent or an isomer, a pharmaceutically acceptable salt, ester, or prodrug thereof.

2. The method of claim 1 wherein the cyclooxygenase-2 selective inhibitor has a selectivity ratio of COX-1 IC₅₀ to COX-2 IC₅₀ not less than about 50.

3. The method of claim 1 wherein the cyclooxygenase-2 selective inhibitor has a selectivity ratio of COX-1 IC₅₀ to COX-2 IC₅₀ not less than about 100.

4. The method of claim 1 wherein the cyclooxygenase-2 selective inhibitor is selected from the group consisting of celecoxib, deracoxib, valdecoxib, rofecoxib, lumiracoxib, etoricoxib, meloxicam, parecoxib, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H)-pyridazinone, 2-[(2,4-dichloro-6-methylphenyl)amino]-5-ethyl-benzeneacetic acid, (3Z)-3-[(4-chlorophenyl)[4-(methylsulfonyl)phenyl]methylene]dihydro-2(3H)-furanone, and (S)-6,8-dichloro-2-(trifluoromethyl)-2H-1-benzopyran-3-carboxylic acid.

5. The method of claim 1 wherein the anticonvulsant agent is selected from the group consisting of phenytoin, fosphenytoin, carbamazepine, valproic acid, felbamate, lamotrigine, topiramate, ethosuximide, clonazepam, diazepam, phenobarbital, mephobarbital, metharbital, primidone, levetiracetam, zonisamide, vigabatrin, gabapentin, tiagabine, clobazam, clorazepate, nitrazepam, lorazepam, methsuximide, phensuximide, ethotoin, mephenyloin, valproate, ethadione, paramethadione, trimethadione, oxcarbazepine, acetazolamide, igmesine, phenacemide, pheneturide, pregabalin, progabalin, ralitoline, remacemide hydrochloride, and rufinamide, or is an isomer, a pharmaceutically acceptable salt, ester, or prodrug thereof.

6. The method of claim 4 wherein the anticonvulsant agent is selected from the group consisting of phenytoin, fosphenytoin, carbamazepine, valproic acid, felbamate, lamotrigine, topiramate, ethosuximide, clonazepam, diazepam, phenobarbital, mephobarbital, metharbital, primidone, levetiracetam, zonisamide, vigabatrin, gabapentin, tiagabine, clobazam, clorazepate, nitrazepam, lorazepam, methsuximide, phensuximide, ethotoin, mephenyloin, valproate, ethadione, paramethadione, trimethadione, oxcarbazepine, acetazolamide, igmesine, phenacemide, pheneturide, pregabalin, progabalin, ralitoline, remacemide hydrochloride, and rufinamide, or is an isomer, a pharmaceutically acceptable salt, ester, or prodrug thereof.

7. The method claim 1 wherein the cyclooxygenase-2 selective inhibitor and anticonvulsant agent are administered substantially simultaneously.

8. The method of claim 1 wherein the cyclooxygenase-2 selective inhibitor and anticonvulsant agent are administered sequentially.

9. The method of claim 1 wherein the cyclooxygenase-2 selective inhibitor is administered to the subject in an amount of about 0.1 to about 20 mg/kg body weight per day.

10. The method of claim 1 wherein the anticonvulsant agent is administered to the subject in an amount of about 50 to about 1000 mg/day.

11. The method of claim 1 wherein the central nervous system disorder or related condition is a seizure or seizure disorder.

12. The method of claim 1 wherein the central nervous system disorder or related condition is a neurogenerative disorder.

13. A method for treating a central nervous system disorder or related condition, the method comprising:
(a) diagnosing a subject in need of treatment for a central nervous system disorder or related condition; and
(b) administering to the subject a cyclooxygenase-2 selective inhibitor or an isomer, a pharmaceutically acceptable salt, ester, or prodrug thereof and an anticonvulsant agent or an isomer, a pharmaceutically acceptable salt, ester, or prodrug thereof, wherein the cyclooxygenase-2 selective inhibitor is a chromene compound, the chromene compound comprising a benzothiopyran, a dihydroquinoline or a dihydronaphthalene.

14. The method of claim 13 wherein the cyclooxygenase-2 selective inhibitor has a selectivity ratio of COX-1 IC₅₀ to COX-2 IC₅₀ not less than about 50.

15. The method of claim 13 wherein the cyclooxygenase-2 selective inhibitor has a selectivity ratio of COX-1 IC₅₀ to COX-2 IC₅₀ not less than about 100.

16. The method of claim 13 wherein the cyclooxygenase-2 selective inhibitor is a compound having the formula: 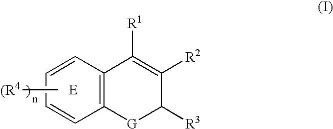
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴ together with the carbon atoms to which it is attached and the remainder of ring E forms a naphthyl radical.

17. The method of claim 13 wherein the cyclooxgyenase-2 selective inhibitor is (S)-6,8-dichloro-2-(trifluoromethyl)-2H-1-benzopyran-3-carboxylic acid.

18. The method of claim 13 wherein the anticonvulsant agent is selected from the group consisting of phenytoin, fosphenytoin, carbamazepine, valproic acid, felbamate, lamotrigine, topiramate, ethosuximide, clonazepam, diazepam, phenobarbital, mephobarbital, metharbital, primidone, levetiracetam, zonisamide, vigabatrin, gabapentin, tiagabine, clobazam, clorazepate, nitrazepam, lorazepam, methsuximide, phensuximide, ethotoin, mephenyloin, valproate, ethadione, paramethadione, trimethadione, oxcarbazepine, acetazolamide, igmesine, phenacemide, pheneturide, pregabalin, progabalin, ralitoline, remacemide hydrochloride, and rufinamide, or is an isomer, a pharmaceutically acceptable salt, ester, or prodrug thereof.

19. A method for treating a central nervous system disorder or related condition, the method comprising:
(a) diagnosing a subject in need of treatment for a central nervous system disorder or related condition; and
(b) administering to the subject a cyclooxygenase-2 selective inhibitor or an isomer, a pharmaceutically acceptable salt, ester, or prodrug thereof and an anticonvulsant agent or an isomer, a pharmaceutically acceptable salt, ester, or prodrug thereof, wherein the cyclooxygenase-2 selective inhibitor is a tricyclic compound, the tricyclic compound containing a benzenesulfonamide or methylsulfonylbenzene moiety.

20. The method of claim 19 wherein the cyclooxygenase-2 selective inhibitor has a selectivity ratio of COX-1 IC₅₀ to COX-2 IC₅₀ not less than about 50.

21. The method of claim 19 wherein the cyclooxygenase-2 selective inhibitor has a selectivity ratio of COX-1 IC₅₀ to COX-2 IC50 not less than about 100.

22. The method of claim 19 wherein the cyclooxygenase-2 selective inhibitor is a compound of the formula: 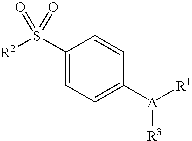
wherein:
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and
R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

23. The method of claim 19 wherein the cyclooxygenase-2 selective inhibitor is selected from the group consisting of celecoxib, valdecoxib, parecoxib, deracoxib, rofecoxib, etoricoxib, and 2-(3,4-difluorophenyl)-4-(3-hydroxy-3-methylbutoxy)-5-[4-(methylsulfonyl)phenyl]-3(2H)-pyridazinone.

24. The method of claim 19 wherein the anticonvulsant agent is selected from the group consisting of phenytoin, fosphenytoin, carbamazepine, valproic acid, felbamate, lamotrigine, topiramate, ethosuximide, clonazepam, diazepam, phenobarbital, mephobarbital, metharbital, primidone, levetiracetam, zonisamide, vigabatrin, gabapentin, tiagabine, clobazam, clorazepate, nitrazepam, lorazepam, methsuximide, phensuximide, ethotoin, mephenyloin, valproate, ethadione, paramethadione, trimethadione, oxcarbazepine, acetazolamide, igmesine, phenacemide, pheneturide, pregabalin, progabalin, ralitoline, remacemide hydrochloride, and rufinamide, or is an isomer, a pharmaceutically acceptable salt, ester, or prodrug thereof.

25. A method for treating a central nervous system disorder or related condition, the method comprising:
(a) diagnosing a subject in need of treatment for a central nervous system disorder or related condition; and
(b) administering to the subject a cyclooxygenase-2 selective inhibitor or an isomer, a pharmaceutically acceptable salt, ester, or prodrug thereof and an anticonvulsant agent or an isomer, a pharmaceutically acceptable salt, ester, or prodrug thereof, wherein the cyclooxygenase-2 selective inhibitor is a phenyl acetic acid compound.

26. The method of claim 25 wherein the cyclooxygenase-2 selective inhibitor has a selectivity ratio of COX-1 IC₅₀ to COX-2 IC₅₀ not less than about 50.

27. The method of claim 25 wherein the cyclooxygenase-2 selective inhibitor has a selectivity ratio of COX-1 IC₅₀ to COX-2 IC₅₀ not less than about 100.

28. The method of claim 25 wherein the cyclooxygenase-2 selective inhibitor is a compound having the formula: 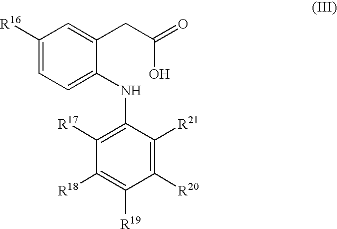
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl; provided, however, that each of R¹⁷, R¹⁸, R¹⁹ and R²⁰ is not fluoro when R¹⁶ is ethyl and R¹⁹ is H.

29. The method of claim 28 wherein:
R¹⁶ is ethyl;
R¹⁷ and R¹⁹ are chloro;
R¹⁸ and R²⁰ are hydrogen; and
R²¹ is methyl.

30. The method of claim 25 wherein the anticonvulsant agent is selected from the group consisting of phenytoin, fosphenytoin, carbamazepine, valproic acid, felbamate, lamotrigine, topiramate, ethosuximide, clonazepam, diazepam, phenobarbital, mephobarbital, metharbital, primidone, levetiracetam, zonisamide, vigabatrin, gabapentin, tiagabine, clobazam, clorazepate, nitrazepam, lorazepam, methsuximide, phensuximide, ethotoin, mephenyloin, valproate, ethadione, paramethadione, trimethadione, oxcarbazepine, acetazolamide, igmesine, phenacemide, pheneturide, pregabalin, progabalin, ralitoline, remacemide hydrochloride, and rufinamide, or is an isomer, a pharmaceutically acceptable salt, ester, or prodrug thereof.

31. A method for treating a central nervous system disorder or related condition, the method comprising:
(a) diagnosing a subject in need of treatment for a central nervous system disorder or related condition; and
(b) administering to the subject a cyclooxygenase-2 selective inhibitor selected from the group consisting of celecoxib, deracoxib, valdecoxib, rofecoxib, lumiracoxib, etoricoxib, parecoxib, 2-(3,4-difluorophenyl)-4-(3-hydroxy-3-methylbutoxy)-5-[4-(methylsulfonyl)phenyl]-3(2H)-pyridazinone, and (S)-6,8-dichloro-2-trifluoromethyl)-2H-1-benzopyran-3-carboxylic acid; and an anticonvulsant agent selected from the group consisting of phenytoin, fosphenytoin, carbamazepine, valproic acid, felbamate, lamotrigine, topiramate, ethosuximide, clonazepam, diazepam, phenobarbital, mephobarbital, metharbital, primidone, levetiracetam, zonisamide, vigabatrin, gabapentin, tiagabine, clobazam, clorazepate, nitrazepam, lorazepam, methsuximide, phensuximide, ethotoin, mephenyloin, valproate, ethadione, paramethadione, trimethadione, oxcarbazepine, acetazolamide, igmesine, phenacemide, pheneturide, pregabalin, progabalin, ralitoline, remacemide hydrochloride, and rufinamide; or an isomer, a pharmaceutically acceptable salt, ester, or prodrug thereof.

32. The method of claim 31 wherein the cyclooxygenase-2 selective inhibitor and anticonvulsant agent are combined and administered in the same dose.

33. The method of claim 31 wherein the cyclooxygenase-2 selective inhibitor and anticonvulsant agent are administered in separate doses.
